SEQUENCE LISTING (87 sequences)

SEQ ID NO: 1 - protein, 98 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 2 - protein, 98 aa, Artificial Sequence
  Description of Artificial Sequence Synthetic polypeptide
  MOD_RES (97)..(97): Arg or Lys
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKXV

SEQ ID NO: 3 - protein, 15 aa, Artificial Sequence
  Description of Artificial Sequence Synthetic peptide
EPKSCDKTHTCPPCP

SEQ ID NO: 4 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 5 - protein, 110 aa, Artificial Sequence
  Description of Artificial Sequence Synthetic polypeptide
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 6 - protein, 110 aa, Artificial Sequence
  Description of Artificial Sequence Synthetic polypeptide
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAK

SEQ ID NO: 7 - protein, 110 aa, Artificial Sequence
  Description of Artificial Sequence Synthetic polypeptide
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAK

SEQ ID NO: 8 - protein, 106 aa, Artificial Sequence
  Description of Artificial Sequence Synthetic polypeptide
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 106 aa, Artificial Sequence
  Description of Artificial Sequence Synthetic polypeptide
  MOD_RES (16)..(16): Glu or Asp
  MOD_RES (18)..(18): Met or Leu
  MOD_RES (91)..(91): Ala or Gly
GQPREPQVYTLPPSRXEXTKNQVSLTCLVKGFYPSDIAVEWESNGQPENNYKTTPPVLDSDGSFFLYSKLTVDKSRWQQGNVFSCSVMHEXLHNHYTQKSLSLSPG

SEQ ID NO: 10 - protein, 106 aa, Artificial Sequence
  Description of Artificial Sequence Synthetic polypeptide
GQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 106 aa, Artificial Sequence
  Description of Artificial Sequence Synthetic polypeptide
  MOD_RES (16)..(16): Glu or Asp
  MOD_RES (18)..(18): Met or Leu
  MOD_RES (91)..(91): Ala or Gly
GQPREPQVYTLPPSRXEXTKNQVSLWCLVKGFYPSDIAVEWESNGQPENNYKTTPPVLDSDGSFFLYSKLTVDKSRWQQGNVFSCSVMHEXLHNHYTQKSLSLSPG

SEQ ID NO: 12 - protein, 106 aa, Artificial Sequence
  Description of Artificial Sequence Synthetic polypeptide
GQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 13 - protein, 106 aa, Artificial Sequence
  Description of Artificial Sequence Synthetic polypeptide
  MOD_RES (16)..(16): Glu or Asp
  MOD_RES (18)..(18): Met or Leu
  MOD_RES (91)..(91): Ala or Gly
GQPREPQVYTLPPSRXEXTKNQVSLSCAVKGFYPSDIAVEWESNGQPENNYKTTPPVLDSDGSFFLVSKLTVDKSRWQQGNVFSCSVMHEXLHNHYTQKSLSLSPG

SEQ ID NO: 14 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 329 aa, Artificial Sequence
  Description of Artificial Sequence Synthetic polypeptide
  MOD_RES (97)..(97): Arg or Lys
  MOD_RES (239)..(239): Glu or Asp
  MOD_RES (241)..(241): Met or Leu
  MOD_RES (314)..(314): Ala or Gly
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEXLHNHYTQKSLSLSPG

SEQ ID NO: 16 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 329 aa, Artificial Sequence
  Description of Artificial Sequence Synthetic polypeptide
  MOD_RES (97)..(97): Arg or Lys
  MOD_RES (239)..(239): Glu or Asp
  MOD_RES (241)..(241): Met or Leu
  MOD_RES (314)..(314): Ala or Gly
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEXLHNHYTQKSLSLSPG

SEQ ID NO: 18 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 329 aa, Artificial Sequence
  Description of Artificial Sequence Synthetic polypeptide
  MOD_RES (97)..(97): Arg or Lys
  MOD_RES (239)..(239): Glu or Asp
  MOD_RES (241)..(241): Met or Leu
  MOD_RES (314)..(314): Ala or Gly
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEXLHNHYTQKSLSLSPG

SEQ ID NO: 20 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 329 aa, Artificial Sequence
  Description of Artificial Sequence Synthetic polypeptide
  MOD_RES (97)..(97): Arg or Lys
  MOD_RES (239)..(239): Glu or Asp
  MOD_RES (241)..(241): Met or Leu
  MOD_RES (314)..(314): Ala or Gly
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEXLHNHYTQKSLSLSPG

SEQ ID NO: 22 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 329 aa, Artificial Sequence
  Description of Artificial Sequence Synthetic polypeptide
  MOD_RES (97)..(97): Arg or Lys
  MOD_RES (239)..(239): Glu or Asp
  MOD_RES (241)..(241): Met or Leu
  MOD_RES (314)..(314): Ala or Gly
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLWCLVKGFYPSDIAVEWESNGQPENNYKTTPPVLDSDGSFFLYSKLTVDKSRWQQGNVFSCSVMHEXLHNHYTQKSLSLSPG

SEQ ID NO: 24 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 329 aa, Artificial Sequence
  Description of Artificial Sequence Synthetic polypeptide
  MOD_RES (97)..(97): Arg or Lys
  MOD_RES (239)..(239): Glu or Asp
  MOD_RES (241)..(241): Met or Leu
  MOD_RES (314)..(314): Ala or Gly
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRXEXTKNQVSLWCLVKGFYPSDIAVEWESNGQPENNYKTTPPVLDSDGSFFLYSKLTVDKSRWQQGNVFSCSVMHEXLHNHYTQKSLSLSPG

SEQ ID NO: 26 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 329 aa, Artificial Sequence
  Description of Artificial Sequence Synthetic polypeptide
  MOD_RES (97)..(97): Arg or Lys
  MOD_RES (239)..(239): Glu or Asp
  MOD_RES (241)..(241): Met or Leu
  MOD_RES (314)..(314): Ala or Gly
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRXEXTKNQVSLWCLVKGFYPSDIAVEWESNGQPENNYKTTPPVLDSDGSFFLYSKLTVDKSRWQQGNVFSCSVMHEXLHNHYTQKSLSLSPG

SEQ ID NO: 28 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 329 aa, Artificial Sequence
  Description of Artificial Sequence Synthetic polypeptide
  MOD_RES (97)..(97): Arg or Lys
  MOD_RES (239)..(239): Glu or Asp
  MOD_RES (241)..(241): Met or Leu
  MOD_RES (314)..(314): Ala or Gly
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLWCLVKGFYPSDIAVEWESNGQPENNYKTTPPVLDSDGSFFLYSKLTVDKSRWQQGNVFSCSVMHEXLHNHYTQKSLSLSPG

SEQ ID NO: 30 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 31 - protein, 329 aa, Artificial Sequence
  Description of Artificial Sequence Synthetic polypeptide
  MOD_RES (97)..(97): Arg or Lys
  MOD_RES (239)..(239): Glu or Asp
  MOD_RES (241)..(241): Met or Leu
  MOD_RES (314)..(314): Ala or Gly
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLSCAVKGFYPSDIAVEWESNGQPENNYKTTPPVLDSDGSFFLVSKLTVDKSRWQQGNVFSCSVMHEXLHNHYTQKSLSLSPG

SEQ ID NO: 32 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 33 - protein, 329 aa, Artificial Sequence
  Description of Artificial Sequence Synthetic polypeptide
  MOD_RES (97)..(97): Arg or Lys
  MOD_RES (239)..(239): Glu or Asp
  MOD_RES (241)..(241): Met or Leu
  MOD_RES (314)..(314): Ala or Gly
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRXEXTKNQVSLSCAVKGFYPSDIAVEWESNGQPENNYKTTPPVLDSDGSFFLVSKLTVDKSRWQQGNVFSCSVMHEXLHNHYTQKSLSLSPG

SEQ ID NO: 34 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 35 - protein, 329 aa, Artificial Sequence
  Description of Artificial Sequence Synthetic polypeptide
  MOD_RES (97)..(97): Arg or Lys
  MOD_RES (239)..(239): Glu or Asp
  MOD_RES (241)..(241): Met or Leu
  MOD_RES (314)..(314): Ala or Gly
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRXEXTKNQVSLSCAVKGFYPSDIAVEWESNGQPENNYKTTPPVLDSDGSFFLVSKLTVDKSRWQQGNVFSCSVMHEXLHNHYTQKSLSLSPG

SEQ ID NO: 36 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 37 - protein, 329 aa, Artificial Sequence
  Description of Artificial Sequence Synthetic polypeptide
  MOD_RES (97)..(97): Arg or Lys
  MOD_RES (239)..(239): Glu or Asp
  MOD_RES (241)..(241): Met or Leu
  MOD_RES (314)..(314): Ala or Gly
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLSCAVKGFYPSDIAVEWESNGQPENNYKTTPPVLDSDGSFFLVSKLTVDKSRWQQGNVFSCSVMHEXLHNHYTQKSLSLSPG

SEQ ID NO: 38 - protein, 216 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 216 aa, Artificial Sequence
  Description of Artificial Sequence Synthetic polypeptide
  MOD_RES (126)..(126): Glu or Asp
  MOD_RES (128)..(128): Met or Leu
  MOD_RES (201)..(201): Ala or Gly
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEXLHNHYTQKSLSLSPG

SEQ ID NO: 40 - protein, 216 aa, Artificial Sequence
  Description of Artificial Sequence Synthetic polypeptide
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 216 aa, Artificial Sequence
  Description of Artificial Sequence Synthetic polypeptide
  MOD_RES (126)..(126): Glu or Asp
  MOD_RES (128)..(128): Met or Leu
  MOD_RES (201)..(201): Ala or Gly
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEXLHNHYTQKSLSLSPG

SEQ ID NO: 42 - protein, 216 aa, Artificial Sequence
  Description of Artificial Sequence Synthetic polypeptide
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 216 aa, Artificial Sequence
  Description of Artificial Sequence Synthetic polypeptide
  MOD_RES (126)..(126): Glu or Asp
  MOD_RES (128)..(128): Met or Leu
  MOD_RES (201)..(201): Ala or Gly
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEXLHNHYTQKSLSLSPG

SEQ ID NO: 44 - protein, 216 aa, Artificial Sequence
  Description of Artificial Sequence Synthetic polypeptide
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 216 aa, Artificial Sequence
  Description of Artificial Sequence Synthetic polypeptide
  MOD_RES (126)..(126): Glu or Asp
  MOD_RES (128)..(128): Met or Leu
  MOD_RES (201)..(201): Ala or Gly
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEXLHNHYTQKSLSLSPG

SEQ ID NO: 46 - protein, 216 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 216 aa, Artificial Sequence
  Description of Artificial Sequence Synthetic polypeptide
  MOD_RES (126)..(126): Glu or Asp
  MOD_RES (128)..(128): Met or Leu
  MOD_RES (201)..(201): Ala or Gly
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLWCLVKGFYPSDIAVEWESNGQPENNYKTTPPVLDSDGSFFLYSKLTVDKSRWQQGNVFSCSVMHEXLHNHYTQKSLSLSPG

SEQ ID NO: 48 - protein, 216 aa, Artificial Sequence
  Description of Artificial Sequence Synthetic polypeptide
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 216 aa, Artificial Sequence
  Description of Artificial Sequence Synthetic polypeptide
  MOD_RES (126)..(126): Glu or Asp
  MOD_RES (128)..(128): Met or Leu
  MOD_RES (201)..(201): Ala or Gly
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRXEXTKNQVSLWCLVKGFYPSDIAVEWESNGQPENNYKTTPPVLDSDGSFFLYSKLTVDKSRWQQGNVFSCSVMHEXLHNHYTQKSLSLSPG

SEQ ID NO: 50 - protein, 216 aa, Artificial Sequence
  Description of Artificial Sequence Synthetic polypeptide
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 216 aa, Artificial Sequence
  Description of Artificial Sequence Synthetic polypeptide
  MOD_RES (126)..(126): Glu or Asp
  MOD_RES (128)..(128): Met or Leu
  MOD_RES (201)..(201): Ala or Gly
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRXEXTKNQVSLWCLVKGFYPSDIAVEWESNGQPENNYKTTPPVLDSDGSFFLYSKLTVDKSRWQQGNVFSCSVMHEXLHNHYTQKSLSLSPG

SEQ ID NO: 52 - protein, 216 aa, Artificial Sequence
  Description of Artificial Sequence Synthetic polypeptide
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 216 aa, Artificial Sequence
  Description of Artificial Sequence Synthetic polypeptide
  MOD_RES (126)..(126): Glu or Asp
  MOD_RES (128)..(128): Met or Leu
  MOD_RES (201)..(201): Ala or Gly
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLWCLVKGFYPSDIAVEWESNGQPENNYKTTPPVLDSDGSFFLYSKLTVDKSRWQQGNVFSCSVMHEXLHNHYTQKSLSLSPG

SEQ ID NO: 54 - protein, 216 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 55 - protein, 216 aa, Artificial Sequence
  Description of Artificial Sequence Synthetic polypeptide
  MOD_RES (126)..(126): Glu or Asp
  MOD_RES (128)..(128): Met or Leu
  MOD_RES (201)..(201): Ala or Gly
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLSCAVKGFYPSDIAVEWESNGQPENNYKTTPPVLDSDGSFFLVSKLTVDKSRWQQGNVFSCSVMHEXLHNHYTQKSLSLSPG

SEQ ID NO: 56 - protein, 216 aa, Artificial Sequence
  Description of Artificial Sequence Synthetic polypeptide
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 57 - protein, 216 aa, Artificial Sequence
  Description of Artificial Sequence Synthetic polypeptide
  MOD_RES (126)..(126): Glu or Asp
  MOD_RES (128)..(128): Met or Leu
  MOD_RES (201)..(201): Ala or Gly
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRXEXTKNQVSLSCAVKGFYPSDIAVEWESNGQPENNYKTTPPVLDSDGSFFLVSKLTVDKSRWQQGNVFSCSVMHEXLHNHYTQKSLSLSPG

SEQ ID NO: 58 - protein, 216 aa, Artificial Sequence
  Description of Artificial Sequence Synthetic polypeptide
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 59 - protein, 216 aa, Artificial Sequence
  Description of Artificial Sequence Synthetic polypeptide
  MOD_RES (126)..(126): Glu or Asp
  MOD_RES (128)..(128): Met or Leu
  MOD_RES (201)..(201): Ala or Gly
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRXEXTKNQVSLSCAVKGFYPSDIAVEWESNGQPENNYKTTPPVLDSDGSFFLVSKLTVDKSRWQQGNVFSCSVMHEXLHNHYTQKSLSLSPG

SEQ ID NO: 60 - protein, 216 aa, Artificial Sequence
  Description of Artificial Sequence Synthetic polypeptide
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 61 - protein, 216 aa, Artificial Sequence
  Description of Artificial Sequence Synthetic polypeptide
  MOD_RES (126)..(126): Glu or Asp
  MOD_RES (128)..(128): Met or Leu
  MOD_RES (201)..(201): Ala or Gly
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLSCAVKGFYPSDIAVEWESNGQPENNYKTTPPVLDSDGSFFLVSKLTVDKSRWQQGNVFSCSVMHEXLHNHYTQKSLSLSPG

SEQ ID NO: 62 - protein, 221 aa, Artificial Sequence
  Description of Artificial Sequence Synthetic polypeptide
CPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 221 aa, Artificial Sequence
  Description of Artificial Sequence Synthetic polypeptide
  MOD_RES (131)..(131): Glu or Asp
  MOD_RES (133)..(133): Met or Leu
  MOD_RES (206)..(206): Ala or Gly
CPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRXEXTKNQVSLWCLVKGFYPSDIAVEWESNGQPENNYKTTPPVLDSDGSFFLYSKLTVDKSRWQQGNVFSCSVMHEXLHNHYTQKSLSLSPG

SEQ ID NO: 64 - protein, 221 aa, Artificial Sequence
  Description of Artificial Sequence Synthetic polypeptide
CPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 65 - protein, 221 aa, Artificial Sequence
  Description of Artificial Sequence Synthetic polypeptide
  MOD_RES (131)..(131): Glu or Asp
  MOD_RES (133)..(133): Met or Leu
  MOD_RES (206)..(206): Ala or Gly
CPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRXEXTKNQVSLWCLVKGFYPSDIAVEWESNGQPENNYKTTPPVLDSDGSFFLYSKLTVDKSRWQQGNVFSCSVMHEXLHNHYTQKSLSLSPG

SEQ ID NO: 66 - protein, 221 aa, Artificial Sequence
  Description of Artificial Sequence Synthetic polypeptide
CPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 67 - protein, 221 aa, Artificial Sequence
  Description of Artificial Sequence Synthetic polypeptide
  MOD_RES (131)..(131): Glu or Asp
  MOD_RES (133)..(133): Met or Leu
  MOD_RES (206)..(206): Ala or Gly
CPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLSCAVKGFYPSDIAVEWESNGQPENNYKTTPPVLDSDGSFFLVSKLTVDKSRWQQGNVFSCSVMHEXLHNHYTQKSLSLSPG

SEQ ID NO: 68 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 69 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 72 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 73 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 74 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 75 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 221 aa, Artificial Sequence
  Description of Artificial Sequence Synthetic polypeptide
CPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 221 aa, Artificial Sequence
  Description of Artificial Sequence Synthetic polypeptide
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 221 aa, Artificial Sequence
  Description of Artificial Sequence Synthetic polypeptide
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 79 - protein, 221 aa, Artificial Sequence
  Description of Artificial Sequence Synthetic polypeptide
CPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 80 - protein, 221 aa, Artificial Sequence
  Description of Artificial Sequence Synthetic polypeptide
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 81 - protein, 221 aa, Artificial Sequence
  Description of Artificial Sequence Synthetic polypeptide
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 82 - protein, 221 aa, Artificial Sequence
  Description of Artificial Sequence Synthetic polypeptide
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 83 - protein, 221 aa, Artificial Sequence
  Description of Artificial Sequence Synthetic polypeptide
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 84 - protein, 221 aa, Artificial Sequence
  Description of Artificial Sequence Synthetic polypeptide
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 85 - protein, 221 aa, Artificial Sequence
  Description of Artificial Sequence Synthetic polypeptide
CPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 86 - protein, 221 aa, Artificial Sequence
  Description of Artificial Sequence Synthetic polypeptide
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 87 - protein, 221 aa, Artificial Sequence
  Description of Artificial Sequence Synthetic polypeptide
CPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

(19) United States
(12) Patent Application Publication
(10) Pub. No.: US 2021/0130495 A1
(43) Pub. Date: May 6, 2021

(54) HETERODIMERIC PROTEINS

(71) Applicant: Agenus Inc., Lexington, MA (US)

(72) Inventors: Dhan Sidhartha Chand, Lexington, MA (US); Zahra Jawad, Lexington, MA (US); Veronica Franciszka Ilkow, Lexington, MA (US)

(21) Appl. No.: 17/035,015

(22) Filed: Sep. 28, 2020

Related U.S. Application Data
(60) Provisional application No. 62/906,918, filed on Sep. 27, 2019.

(51) Int. Cl. C07K 16/42

(52) U.S. Cl. CPC C07K 16/4283; C07K 2319/30; C07K 2317/14; C07K 2317/524; C07K 2317/526; C07K 2317/522

(57) ABSTRACT

Provided herein are heterodimeric proteins (e.g., multispecific antibodies) that exhibit enhanced binding to human Fc gamma receptor IIIA (FcγRIIIA) relative to naturally occurring antibodies and retain good manufacturability. Such heterodimeric proteins are particular useful as multispecific binding proteins (e.g., multispecific antibodies). Also provided are pharmaceutical compositions comprising these heterodimeric proteins, nucleic acids encoding these heterodimeric proteins, and expression vectors and host cells for making these heterodimeric proteins.

### RELATED APPLICATIONS

This application claims the benefit of U.S. Provisional Application No. 62/906,918, filed Sep. 27, 2019, which is incorporated by reference herein in its entirety.

### 1. FIELD

The instant disclosure relates to heterodimeric proteins (e.g., multispecific antibodies) and methods of making the same.

### 2. BACKGROUND

Multispecific binding molecules (e.g., multispecific antibodies) comprising two or more binding specificities hold great promise as therapeutics because of the ability of these molecules to modulate the activity of multiple targets in vivo. One common method for producing multispecific antibodies is to heterodimerize the heavy chains of two different antibodies. To facilitate this heterodimerization, the CH3 domains of the two antibodies are engineered to contain complementary sets of mutations that result in a preference for heavy chain heterodimerization relative to homodimerization (see e.g., Ridgway J. B. et al., 1996, Protein Eng., 9:617-621; and U.S. Pat. No. 9,409,989, each of which is herein incorporated by reference in its entirety).

In certain instances, it is desirable to enhance the binding of the Fc region of a multispecific antibody to human Fc gamma receptor IIIA (FcγRIIIA) relative to that of the Fc region of a corresponding naturally occurring antibody. One method of achieving this enhanced FcγRIIIA binding is to engineer the Fc region of the multispecific antibody to comprise specific amino acid mutations at amino acid positions 239, 330, and 332 of the Fc region, according to the EU index (see e.g., Lazar, G. A. et al., 2006, PNAS, 103(11):4005-4010). However, Applicant has discovered that combining these Fc mutations with heterodimerization-enhancing Fc mutations at amino acid positions 366, 368 and 407, according to the EU index, can negatively impact the manufacturability of multispecific antibodies comprising these mutations.

Accordingly, there is a need in the art for improved multispecific antibodies that exhibit enhanced binding to FcγRIIIA, while retaining good manufacturability.

### 3. SUMMARY

The instant disclosure provides heterodimeric proteins (e.g., multispecific antibodies) that exhibit enhanced binding to FcγRIIIA relative to a corresponding naturally occurring antibody and exhibit good manufacturability. The heterodimeric proteins generally comprise a first Fc polypeptide comprising aspartate, glutamate, and tryptophan at amino acid positions 239, 332, and 366, respectively; and a second Fc polypeptide comprising aspartate, serine, alanine, and valine at amino acid positions 239, 366, 368, and 407, respectively, but not comprising glutamate at amino acid position 332, wherein the amino acid positions are numbered according to the EU index. In certain embodiments, the heterodimeric proteins comprise a first Fc polypeptide comprising aspartate, leucine, glutamate, and tryptophan at amino acid positions 239, 330, 332, and 366, respectively; and a second Fc polypeptide comprising aspartate, serine, alanine, and valine at amino acid positions 239, 366, 368, and 407, respectively, but not comprising leucine and glutamate at amino acid positions 330 and 332, respectively, wherein the amino acid positions are numbered according to the EU index. Such heterodimeric proteins are particularly useful as multispecific binding proteins (e.g., multispecific antibodies). Also provided are pharmaceutical compositions comprising these heterodimeric proteins, nucleic acids encoding these heterodimeric proteins, and expression vectors and host cells for making these heterodimeric proteins. Without wishing to be bound by theory, Applicant believes that the second Fc polypeptide of the heterodimeric proteins disclosed herein has improved thermal stability relative to a corresponding Fc polypeptide comprising mutations at amino acid positions 239, 330, 332, 366, 368, and 407, numbered according to the EU index.

Accordingly, in one aspect the instant disclosure provides heterodimeric proteins.

In certain embodiments, the heterodimeric protein comprises a first Fc polypeptide and a second Fc polypeptide, wherein: the first Fc polypeptide comprises aspartate, glutamate, and tryptophan at amino acid positions 239, 332, and 366, respectively; and the second Fc polypeptide comprises aspartate, serine, alanine, and valine at amino acid positions 239, 366, 368, and 407, respectively, but does not comprise glutamate at amino acid position 332, wherein the amino acid positions are numbered according to the EU index. In certain embodiments, the first Fc polypeptide further comprises leucine at amino acid position 330, wherein the amino acid positions are numbered according to the EU index. In certain embodiments, the second Fc polypeptide does not comprise leucine at amino acid position 330, wherein the amino acid positions are numbered according to the EU index.

In certain embodiments, the first Fc polypeptide comprises a first antigen-binding moiety and/or the second Fc polypeptide comprises a second antigen-binding moiety. In certain embodiments, the first and/or second antigen-binding moiety comprises an antibody variable domain, an extracellular domain of a cell surface receptor, a soluble T cell receptor (e.g., a T cell receptor lacking endogenous transmembrane and cytoplasmic regions), or a ligand. In certain embodiments, the first and/or second antigen-binding moiety comprises a VH, a VL, a VHH, a VH/VL pair, an scFv, a diabody, and/or a Fab. In certain embodiments, the cell surface receptor is a tumor necrosis factor superfamily receptor, a vascular endothelial growth factor receptor, or a transforming growth factor receptor. In certain embodiments, the soluble T cell receptor comprises an extracellular, antigen-binding portion of a T cell receptor stabilized by one or more engineered disulphide bonds. In certain embodiments, the soluble T cell receptor comprises a single chain T cell receptor comprising variable regions of a T cell receptor linked together by a flexible linker (e.g., a peptide linker). In certain embodiments, the ligand is a hormone or growth factor. In certain embodiments, the first and second antigen-binding moieties specifically bind to the same or different target molecules.

In certain embodiments, the first and/or second Fc polypeptide comprises a CH1 domain, hinge region, CH2 domain and/or CH3 domain of human IgG₁, IgG₂, IgG₃, IgG₄, IgA₁, or IgA₂. In certain embodiments, the first and/or second Fc polypeptide comprises an antibody heavy chain. In certain embodiments, the antibody heavy chain lacks a CH1 domain and/or a portion of a hinge region. In certain embodiments, the antibody heavy chain is a full-length antibody heavy chain. In certain embodiments, the antibody heavy chain is a human IgG₁, IgG₂, IgG₃, IgG₄, IgA₁, or IgA₂ heavy chain.

In certain embodiments, the first and/or second Fc polypeptide further comprises an antibody light chain. In certain embodiments, the antibody light chain is a human kappa or lambda light chain.

In certain embodiments, the first Fc polypeptide comprises a first half-antibody comprising a first antibody heavy chain and a first antibody light chain; and/or the second Fc polypeptide comprises a second half-antibody comprising a second antibody heavy chain and a second antibody light chain. In certain embodiments, the first Fc polypeptide comprises a first half-antibody comprising a first antibody heavy chain and a first antibody light chain; and the second Fc polypeptide comprises a second half-antibody comprising a second antibody heavy chain and a second antibody light chain. In certain embodiments, the first and second half-antibodies bind to different target molecules or to different regions of the same target molecule.

In certain embodiments, the first Fc polypeptide comprises an amino acid sequence that is at least 75% identical (optionally at least 76, 77, 78, 79, 80, 81, 82, 83, 84, 85, 86, 87, 88, 89, 90, 91, 92, 93, 94, 95, 96, 97, 98, 99, or 100% identical) to an amino acid sequence selected from the group consisting of SEQ ID NO: 1-3, 6-7, 10-11, 24-27, 48-51, and 62-65; and/or the second Fc polypeptide comprises an amino acid sequence that is at least 75% identical (optionally at least 75, 76, 77, 78, 79, 80, 81, 82, 83, 84, 85, 86, 87, 88, 89, 90, 91, 92, 93, 94, 95, 96, 97, 98, 99, or 100% identical) to an amino acid sequence selected from the group consisting of SEQ ID NO: 1-3, 5, 12-13, 36-37, 60-61, and 66-67.

In certain embodiments, the first Fc polypeptide comprises an amino acid sequence that is at least 75% identical (optionally at least 76, 77, 78, 79, 80, 81, 82, 83, 84, 85, 86, 87, 88, 89, 90, 91, 92, 93, 94, 95, 96, 97, 98, 99, or 100% identical) to an amino acid sequence selected from the group consisting of SEQ ID NO: 1-3, 6-7, 10-11, 24-27, 48-51, and 62-65; and the second Fc polypeptide comprises an amino acid sequence that is at least 75% identical (optionally at least 75, 76, 77, 78, 79, 80, 81, 82, 83, 84, 85, 86, 87, 88, 89, 90, 91, 92, 93, 94, 95, 96, 97, 98, 99, or 100% identical) to an amino acid sequence selected from the group consisting of SEQ ID NO: 1-3, 5, 12-13, 36-37, 60-61, and 66-67.

In certain embodiments, the first Fc polypeptide comprises an amino acid sequence selected from the group consisting of SEQ ID NOs: 1-3, 6-7, 10-11, 24-27, 48-51, and 62-65. In certain embodiments, the second Fc polypeptide comprises an amino acid sequence selected from the group consisting of SEQ ID NOs: 1-3, 5, 12-13, 36-37, 60-61, and 66-67.

In certain embodiments, the first Fc polypeptide and second Fc polypeptide comprise amino acid sequences that are at least 75% identical (e.g., at least 75, 76, 77, 78, 79, 80, 81, 82, 83, 84, 85, 86, 87, 88, 89, 90, 91, 92, 93, 94, 95, 96, 97, 98, 99, or 100% identical) to the amino acid sequences of SEQ ID NOs: 24 and 36; 24 and 37; 25 and 36; 25 and 37; 26 and 36; 26 and 37; 27 and 36; 27 and 37; 48 and 60; 48 and 61; 49 and 60; 49 and 61; 50 and 60; 50 and 61; 51 and 60; 51 and 61; 62 and 66; 62 and 67; 63 and 66; 63 and 67; 64 and 66; 64 and 67; 65 and 66; or 65 and 67, respectively. In certain embodiments, the first Fc polypeptide and second Fc polypeptide comprise the amino acid sequences of SEQ ID NOs: 24 and 36; 24 and 37; 25 and 36; 25 and 37; 26 and 36; 26 and 37; 27 and 36; 27 and 37; 48 and 60; 48 and 61; 49 and 60; 49 and 61; 50 and 60; 50 and 61; 51 and 60; 51 and 61; 62 and 66; 62 and 67; 63 and 66; 63 and 67; 64 and 66; 64 and 67; 65 and 66; or 65 and 67, respectively.

In certain embodiments, the first Fc polypeptide comprises the amino acid sequence set forth in SEQ ID NO: 51 and the second Fc polypeptide comprises the amino acid sequence set forth in SEQ ID NO: 61. In certain embodiments, the first Fc polypeptide comprises the amino acid sequence set forth in SEQ ID NO: 50 and the second Fc polypeptide comprises the amino acid sequence set forth in SEQ ID NO: 60. In certain embodiments, the first Fc polypeptide comprises the amino acid sequence set forth in SEQ ID NO: 51 and the second Fc polypeptide comprises the amino acid sequence set forth in SEQ ID NO: 60. In certain embodiments, the first Fc polypeptide comprises the amino acid sequence set forth in SEQ ID NO: 50 and the second Fc polypeptide comprises the amino acid sequence set forth in SEQ ID NO: 61. In certain embodiments, the first Fc polypeptide comprises the amino acid sequence set forth in SEQ ID NO: 49 and the second Fc polypeptide comprises the amino acid sequence set forth in SEQ ID NO: 61. In certain embodiments, the first Fc polypeptide comprises the amino acid sequence set forth in SEQ ID NO: 48 and the second Fc polypeptide comprises the amino acid sequence set forth in SEQ ID NO: 60. In certain embodiments, the first Fc polypeptide comprises the amino acid sequence set forth in SEQ ID NO: 49 and the second Fc polypeptide comprises the amino acid sequence set forth in SEQ ID NO: 60. In certain embodiments, the first Fc polypeptide comprises the amino acid sequence set forth in SEQ ID NO: 48 and the second Fc polypeptide comprises the amino acid sequence set forth in SEQ ID NO: 61. In certain embodiments, the first Fc polypeptide comprises the amino acid sequence set forth in SEQ ID NO: 65 and the second Fc polypeptide comprises the amino acid sequence set forth in SEQ ID NO: 67. In certain embodiments, the first Fc polypeptide comprises the amino acid sequence set forth in SEQ ID NO: 64 and the second Fc polypeptide comprises the amino acid sequence set forth in SEQ ID NO: 66. In certain embodiments, the first Fc polypeptide comprises the amino acid sequence set forth in SEQ ID NO: 65 and the second Fc polypeptide comprises the amino acid sequence set forth in SEQ ID NO: 66. In certain embodiments, the first Fc polypeptide comprises the amino acid sequence set forth in SEQ ID NO: 64 and the second Fc polypeptide comprises the amino acid sequence set forth in SEQ ID NO: 67. In certain embodiments, the first Fc polypeptide comprises the amino acid sequence set forth in SEQ ID NO: 63 and the second Fc polypeptide comprises the amino acid sequence set forth in SEQ ID NO: 67. In certain embodiments, the first Fc polypeptide comprises the amino acid sequence set forth in SEQ ID NO: 62 and the second Fc polypeptide comprises the amino acid sequence set forth in SEQ ID NO: 66. In certain embodiments, the first Fc polypeptide comprises the amino acid sequence set forth in SEQ ID NO: 63 and the second Fc polypeptide comprises the amino acid sequence set forth in SEQ ID NO: 66. In certain embodiments, wherein the first Fc polypeptide comprises the amino acid sequence set forth in SEQ ID NO: 62 and the second Fc polypeptide comprises the amino acid sequence set forth in SEQ ID NO: 67. In certain embodiments, the first Fc polypeptide comprises the amino acid sequence set forth in SEQ ID NO: 27 and the second Fc polypeptide comprises the amino acid sequence set forth in SEQ ID NO: 37. In certain embodiments, the first Fc polypeptide comprises the amino acid sequence set forth in SEQ ID NO: 26 and the second Fc polypeptide comprises the amino acid sequence set forth in SEQ ID NO: 36. In certain embodiments, the first Fc polypeptide comprises the amino acid sequence set forth in SEQ ID NO: 27 and the second Fc polypeptide comprises the amino acid sequence set forth in SEQ ID NO: 36. In certain embodiments, the first Fc polypeptide comprises the amino acid sequence set forth in SEQ ID NO: 26 and the second Fc polypeptide comprises the amino acid sequence set forth in SEQ ID NO: 37. In certain embodiments, the first Fc polypeptide comprises the amino acid sequence set forth in SEQ ID NO: 25 and the second Fc polypeptide comprises the amino acid sequence set forth in SEQ ID NO: 37. In certain embodiments, the first Fc polypeptide comprises the amino acid sequence set forth in SEQ ID NO: 24 and the second Fc polypeptide comprises the amino acid sequence set forth in SEQ ID NO: 36. In certain embodiments, the first Fc polypeptide comprises the amino acid sequence set forth in SEQ ID NO: 25 and the second Fc polypeptide comprises the amino acid sequence set forth in SEQ ID NO: 36. In certain embodiments, the first Fc polypeptide comprises the amino acid sequence set forth in SEQ ID NO: 24 and the second Fc polypeptide comprises the amino acid sequence set forth in SEQ ID NO: 37.

In another aspect the instant disclosure provides pharmaceutical compositions comprising the heterodimeric proteins disclosed herein. In certain embodiments, the pharmaceutical composition comprises a heterodimeric protein disclosed herein and a pharmaceutically acceptable carrier or excipient.

In another aspect the instant disclosure provides isolated polynucleotides encoding the Fc polypeptides disclosed herein, vectors comprising the polynucleotides, and recombinant host cells comprising the polynucleotides, vectors, and Fc polypeptides disclosed herein. In certain embodiments, the isolated polynucleotide encodes the first and second Fc polypeptide of any of any of the heterodimeric proteins disclosed herein. In certain embodiments, the vector comprises a polynucleotide encoding the first and second Fc polypeptide of any of any of the heterodimeric proteins disclosed herein.

In certain embodiments, the recombinant host cell comprises:
a) a polynucleotide encoding the first and second Fc polypeptide of any of any of the heterodimeric proteins disclosed herein;
b) a vector comprising a polynucleotide encoding the first and second Fc polypeptide of any of any of the heterodimeric proteins disclosed herein;
c) a first polynucleotide encoding the first Fc polypeptide of any of the heterodimeric proteins disclosed herein, and a second polynucleotide encoding the second Fc polypeptide of any of the heterodimeric proteins disclosed herein;
d) a first vector comprising a first polynucleotide encoding the first Fc polypeptide of any of the heterodimeric proteins disclosed herein, and a second vector comprising a second polynucleotide encoding the second Fc polypeptide of any of the heterodimeric proteins disclosed herein;
e) a first polynucleotide encoding a first antibody heavy chain of a first Fc polypeptide of a heterodimeric protein disclosed herein, and a second polynucleotide encoding the second antibody heavy chain of a second Fc polypeptide of a heterodimeric protein disclosed herein, and optionally a third polynucleotide encoding a first antibody light chain of the first Fc polypeptide, and/or a fourth polynucleotide encoding a second antibody light chain of the second Fc polypeptide;
wherein the first Fc polypeptide comprises a first half-antibody comprising a first antibody heavy chain and a first antibody light chain; and the second Fc polypeptide comprises a second half-antibody comprising a second antibody heavy chain and a second antibody light chain; or
f) a first vector comprising a first polynucleotide encoding the first antibody heavy chain of a first Fc polypeptide of a heterodimeric protein disclosed herein, and a second vector comprising a second polynucleotide encoding a second antibody heavy chain of a second Fc polypeptide of a heterodimeric protein disclosed herein, and optionally a third vector comprising a third polynucleotide encoding the first antibody light chain of the first Fc polypeptide, and/or a fourth vector comprising a fourth polynucleotide encoding the second antibody light chain of the second Fc polypeptide,
wherein the first Fc polypeptide comprises a first half-antibody comprising a first antibody heavy chain and a first antibody light chain; and the second Fc polypeptide comprises a second half-antibody comprising a second antibody heavy chain and a second antibody light chain.

In another aspect the instant disclosure provides methods for producing the heterodimeric proteins described herein.

In certain embodiments, the method comprises expressing in a cell a first polynucleotide encoding the first Fc polypeptide of any of the heterodimeric proteins disclosed herein; and a second polynucleotide encoding the second Fc polypeptide of any of the heterodimeric proteins disclosed herein, under conditions whereby the heterodimeric protein is produced.

In certain embodiments, the method comprises expressing in a cell:
a) a first polynucleotide encoding the first antibody heavy chain of a first Fc polypeptide of a heterodimeric protein disclosed herein;
b) a second polynucleotide encoding the second antibody heavy chain of a second Fc polypeptide of a heterodimeric protein disclosed herein;
c) a third polynucleotide encoding the first antibody light chain of the first Fc polypeptide; and
d) a fourth polynucleotide encoding the second antibody light chain of the second Fc polypeptide,
under conditions whereby the heterodimeric protein is produced,
wherein the first Fc polypeptide comprises a first half-antibody comprising a first antibody heavy chain and a first antibody light chain; and the second Fc polypeptide comprises a second half-antibody comprising a second antibody heavy chain and a second antibody light chain.

In certain embodiments, the method comprises:
a) expressing in a first cell a first polynucleotide encoding the first Fc polypeptide of any of the heterodimeric proteins disclosed herein, under conditions whereby the first Fc polypeptide is produced;
b) expressing in a second cell a second polynucleotide encoding the second Fc polypeptide of any of the heterodimeric proteins disclosed herein, under conditions whereby the second Fc polypeptide is produced; and
c) contacting the first and the second Fc polypeptides produced in steps (a) and (b), under conditions whereby the first Fc polypeptide and the second Fc polypeptide heterodimerize to produce the heterodimeric protein.

In certain embodiments, the method comprises:
a) expressing in a first cell a first polynucleotide encoding the first antibody heavy chain and second polynucleotide encoding the first antibody light chain of a first Fc polypeptide of a heterodimeric protein disclosed herein, under conditions whereby the first Fc polypeptide is produced;
b) expressing in a second cell a third polynucleotide encoding the second antibody heavy chain and a fourth polynucleotide encoding the second antibody light chain a second Fc polypeptide of a heterodimeric protein disclosed herein, under conditions whereby the second Fc polypeptide is produced; and
c) contacting the first and the second Fc polypeptides produced in steps (a) and (b), under conditions whereby the first Fc polypeptide and the second Fc polypeptide heterodimerize to produce the heterodimeric protein,
wherein the first Fc polypeptide comprises a first half-antibody comprising a first antibody heavy chain and a first antibody light chain; and the second Fc polypeptide comprises a second half-antibody comprising a second antibody heavy chain and a second antibody light chain.

In certain embodiments, the method comprises contacting the first Fc polypeptide and the second Fc polypeptide of the heterodimeric protein of any of the heterodimeric proteins disclosed herein under conditions whereby the first Fc polypeptide and the second Fc polypeptide heterodimerize to produce the heterodimeric protein.

### 4. BRIEF DESCRIPTION OF THE DRAWINGS

FIG. 1 is a graph showing expression levels of a subset of the Fc polypeptides described in Table 3.

FIGS. 2A and 2B are graphs showing the thermal stability of heterodimeric proteins BA111, BA112, BA113, and BA114 (FIG. 2A; “anti-Target 1 antibody”) and BA115, BA116, BA117, and BA118 (FIG. 2B; “anti-Target 2 antibody”) as determined by measuring thermal unfolding transition.

FIG. 3 is a graph showing the binding of heterodimeric proteins BA111, BA112, BA113, BA114, BA119, and an IgG₁isotype control antibody to Jurkat cells engineered to express high levels of cell surface Target 1. In each case, the extent of binding to Jurkat cells, as assessed by median fluorescence intensity (MFI), is plotted against the concentration of the heterodimeric protein (“antibody”) incubated with the cells.

FIG. 4 is a graph showing the binding of heterodimeric proteins BA115, BA116, BA117, BA118, BA120, and an IgG₁isotype control antibody to CHO cells engineered to express high levels of cell surface Target 2. In each case, the extent of binding to CHO cells, as assessed by median fluorescence intensity (MFI), is plotted against the concentration of the heterodimeric protein (“antibody”) incubated with the cells.

FIG. 5 is a graph showing the results of IL-2-luciferase reporter assays measuring the relative amount of T cell activation resulting from Target 1 blockade using the heterodimeric proteins BA111, BA112, BA113, BA114, BA119, or an IgG₁isotype control antibody. Luciferase expression (measured in average RLU), a surrogate marker for IL-2 gene activation, is plotted against heterodimeric protein (“antibody”) concentration.

FIG. 6 is a graph showing the results of IL-2-luciferase reporter assays measuring the relative amount of T cell activation resulting from Target 2 blockade using the heterodimeric proteins BA115, BA116, BA117, BA118, BA120, or an IgG₁isotype control antibody. Luciferase expression (measured in average RLU), a surrogate marker for IL-2 gene activation, is plotted against heterodimeric protein (“antibody”) concentration.

FIGS. 7A-7P are graphs showing the ability of heterodimeric proteins BA111, BA112, BA113, BA114, BA119, and Reference Homodimeric Protein 2 to induced IL-2 secretion by SEA-stimulated PBMCs in three different donors, Donor 1 (FIGS. 7A-7D and FIGS. 7I-7L), Donor 2 (FIGS. 7E-7H), and Donor 3 (FIGS. 7M-7P). A homodimeric isotype protein, which comprises S239D/A330L/I332E mutations (“Fc enhanced”) was used as an isotype control. IL-2 concentration is plotted against heterodimeric protein (“antibody”) concentration.

FIGS. 8A-8X are graphs showing the ability of BA115, BA116, BA117, BA118, and Reference Homodimeric Protein 1 to induce IL-2 secretion by SEA-stimulated PBMCs in three different donors, Donor 1 (FIGS. 8A-8D and 8M-8P), Donor 2 (FIGS. 8I-8L and 8U-8X), and Donor 3 (FIGS. 8E-8H and 8Q-8T). A homodimeric isotype protein, which comprises S239D/A330L/I332E mutations (“Fc enhanced”) was used as an isotype control. IL-2 concentration is plotted against heterodimeric protein (“antibody”) concentration.

FIGS. 9A and 9B are a set of graphs showing the binding of heterodimeric proteins BA111, BA112, BA113, and BA114 to CHO cells expressing cell surface human FcγRIIIA V/V (FIG. 9A) or CHO cells expressing cell surface human FcγRIIIA F/F (FIG. 9B). The level of heterodimeric protein binding to the cells, as assessed by the geometric mean fluorescence intensity (MFI), was plotted against the concentrations of heterodimeric protein (“Ab”) incubated with the cells.

FIGS. 10A and 10B are a set of graphs showing the binding of heterodimeric protein BA115, BA116, BA117, and BA118 to CHO cells expressing cell surface human FcγRIIIA V/V (FIG. 10A) or CHO cells expressing cell surface human FcγRIIIA F/F (FIG. 10B). The level of heterodimeric protein binding to the cells, as assessed by the geometric mean fluorescence intensity (MFI), was plotted against the concentrations of heterodimeric protein (“Ab”) incubated with the cells.

FIGS. 11A-11G are a set of graphs showing binding of anti-target 1 heterodimeric proteins to CHO cells expressing cell surface human FcγRIIIA F/F. The level of heterodimeric protein binding to the cells, as assessed by the geometric mean fluorescence intensity (MFI), was plotted against the concentrations of heterodimeric protein incubated with the cells.

FIGS. 12A-12G are a set of graphs showing binding of anti-target 2 heterodimeric proteins to CHO cells expressing cell surface human FcγRIIIA F/F. The level of heterodimeric protein binding to the cells, as assessed by the geometric mean fluorescence intensity (MFI), was plotted against the concentrations of heterodimeric protein incubated with the cells.

### 5. DETAILED DESCRIPTION

Provided herein are heterodimeric proteins (e.g., multispecific antibodies) that exhibit enhanced binding to FcγRIIIA relative to a corresponding naturally occurring antibody and exhibit good manufacturability. The heterodimeric proteins generally comprise a first Fc polypeptide comprising aspartate, glutamate, and tryptophan at amino acid positions 239, 332, and 366, respectively; and a second Fc polypeptide comprising aspartate, serine, alanine, and valine at amino acid positions 239, 366, 368, and 407, respectively, but not comprising glutamate at amino acid position 332, wherein the amino acid positions are numbered according to the EU index. In certain embodiments, the heterodimeric proteins comprise a first Fc polypeptide comprising aspartate, leucine, glutamate, and tryptophan at amino acid positions 239, 330, 332, and 366, respectively; and a second Fc polypeptide comprising aspartate, serine, alanine, and valine at amino acid positions 239, 366, 368, and 407, respectively, but not comprising leucine and glutamate at amino acid positions 330 and 332, respectively, wherein the amino acid positions are numbered according to the EU index. Such heterodimeric proteins are particularly useful as multispecific binding proteins (e.g., multispecific antibodies). Also provided are pharmaceutical compositions comprising these heterodimeric proteins, nucleic acids encoding these heterodimeric proteins, and expression vectors and host cells for making these heterodimeric proteins.

### 5.1 Definitions

As used herein, the term “heterodimeric protein” refers to a protein comprising a covalently or non-covalently linked dimer of two non-identical Fc polypeptides.

As used herein, the term “Fc polypeptide” refers to a polypeptide comprising a CH2 domain and a CH3 domain, wherein the C-terminus of the CH2 domain is linked (directly or indirectly) to the N-terminus of the CH3 domain. The term “Fc polypeptide” includes an antibody heavy chain linked to an antibody light chain by disulphide bonds (e.g., to form a half-antibody).

As used herein, the term “CH1 domain” refers to the first constant domain of an antibody heavy chain (e.g., amino acid positions 118-215 of human IgG₁, according to the EU index). The term includes naturally occurring CH1 domains and engineered variants of naturally occurring CH1 domains (e.g., CH1 domains comprising one or more amino acid insertions, deletions, substitutions, or modifications relative to a naturally occurring CH1 domain).

As used herein, the term “CH2 domain” refers to the second constant domain of an antibody heavy chain (e.g., amino acid positions 231-340 of human IgG₁, according to the EU index). The term includes naturally occurring CH2 domains and engineered variants of naturally occurring CH2 domains (e.g., CH2 domains comprising one or more amino acid insertions, deletions, substitutions, or modifications relative to a naturally occurring CH2 domain).

As used herein, the term “CH3 domain” refers to the third constant domain of an antibody heavy chain (e.g., amino acid positions 341-447 of human IgG₁, according to the EU index). The term includes naturally occurring CH3 domains and engineered variants of naturally occurring CH2 domains (e.g., CH3 domains comprising one or more amino acid insertions, deletions, substitutions, or modifications relative to a naturally occurring CH3 domain).

As used herein, the term “hinge region” refers to the portion of an antibody heavy chain comprising the cysteine residues (e.g., the cysteine residues at amino acid positions 226 and 229 of human IgG₁, according to the EU index) that mediate disulphide bonding between two heavy chains in an intact antibody. The term includes naturally occurring hinge regions and engineered variants of naturally occurring hinge regions (e.g., hinge regions comprising one or more amino acid insertions, deletions, substitutions, or modifications relative to a naturally occurring hinge regions). An exemplary full-length IgG₁hinge region comprises amino acid positions 216-230 of human IgG₁, according to the EU index.

As used herein, the term “EU index” refers to the EU numbering convention for the constant regions of an antibody, as described in Edelman, G M. et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety. All numbering of amino acid positions of the Fc polypeptides, or fragments thereof, used herein is according to the EU index.

As used herein, the term “antigen-binding moiety” refers to a molecule that specifically binds to an antigen as such binding is understood by one skilled in the art. For example, an antigen-binding moiety that specifically binds to an antigen binds to other molecules, generally with lower affinity as determined by, e.g., immunoassays, BlAcore®, KinExA 3000 instrument (Sapidyne Instruments, Boise, Id.), or other assays known in the art. In certain embodiments, an antigen-binding moiety that specifically binds to an antigen binds to the antigen with a K_{A} that is at least 2 logs (e.g., factors of 10), 2.5 logs, 3 logs, 4 logs or greater than the K_{A} when the molecule binds non-specifically to another antigen.

As used herein, the terms “antibody” and “antibodies” include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, Fab fragments, F(ab′)₂ fragments, disulphide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA or IgY), any class (e.g., IgG₁, IgG₂, IgG₃, IgG₄, IgA₁ or IgA₂), or any subclass (e.g., IgG₂ₐ or IgG_{2b}) of immunoglobulin molecule. In certain embodiments, antibodies described herein are IgG antibodies, or a class (e.g., human IgG₁or IgG₄) or subclass thereof.

As used herein, the the terms “VH” and “VL” refer to antibody heavy and light chain variable domain, respectively, as described in Kabat et al., (1991) Sequences of Proteins of Immunological Interest (NIH Publication No. 91-3242, Bethesda), which is herein incorporated by reference in its entirety.

As used herein, the the term “VHH” refers to the heavy chain variable domain of a camelid heavy chain-only antibody (HCAb) and humanized variants thereof, as described in Hamers-Casterman C. et al., Nature (1993) 363:446-8.10.1038/363446a0, which is incorporated by reference herein in its entirety.

As used herein, the term “VH/VL Pair” refers to a combination of a VH and a VL that together form the binding site for an antigen.

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG₁, IgG₂, IgG₃, and IgG₄.

As used herein, the term “full-length antibody heavy chain” refers to an antibody heavy chain comprising, from N to C terminal, a VH, a CH1 region, a hinge region, a CH2 domain and a CH3 domain.

As used herein, the term “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain.

As used herein, the term “half-antibody” refers to an antibody heavy chain and an antibody light chain linked to one another in the same manner as the heavy and light chains of an intact, wild-type immunoglobulin. In certain embodiments, a half-antibody is produced by the reduction of the inter-heavy chain disulphide bonds of the hinge region of a full-length antibody. In certain embodiments, a half-antibody is produced by expression of an Fc polypeptide disclosed herein in a cell.

### 5.2 Heterodimeric Proteins

In one aspect, the instant disclosure provides heterodimeric proteins comprising a first and a second Fc polypeptide, wherein: the first Fc polypeptide comprises the amino acids aspartate, glutamate, and tryptophan at amino acid positions 239, 332, and 366, respectively; and the second Fc polypeptide comprises the amino acids aspartate, serine, alanine, and valine at amino acid positions 239, 366, 368, and 407, respectively, but does not comprise the amino acids glutamate at amino acid position 332, wherein the amino acid positions are numbered according to the EU index in each case. In certain embodiments, the first Fc polypeptide further comprises the amino acid leucine at amino acid position 330, wherein the amino acid positions are numbered according to the EU index. In certain embodiments, the first Fc polypeptide further comprises the amino acid leucine at amino acid position 330 and the second Fc polypeptide does not comprise the amino acid leucine at amino acid position 330, wherein the amino acid positions are numbered according to the EU index.

In another aspect, the instant disclosure provides heterodimeric proteins comprising a first and a second Fc polypeptide, wherein: the first Fc polypeptide comprises the amino acid tryptophan at amino acid position 366, but does not comprise the amino acids aspartate, leucine, and glutamate at amino acid positions 239, 330, and 332, respectively; and the second Fc polypeptide comprises:

(a) the amino acids serine, alanine, and valine at amino acid positions 366, 368, and 407, respectively, but does not comprise the amino acids aspartate, leucine, and glutamate at amino acid positions 239, 330, and 332, respectively;

(b) the amino acids aspartate, leucine, serine, alanine, and valine at amino acid positions 239, 330, 366, 368, and 407, respectively, but does not comprise the amino acids glutamate at amino acid position 332;

(c) the amino acids leucine, glutamate, serine, alanine, and valine at amino acid positions 330, 332, 366, 368, and 407, respectively, but does not comprise the amino acid aspartate at amino acid position 239;

(d) the amino acids aspartate, glutamate, serine, alanine, and valine at amino acid positions 239, 332, 366, 368, and 407, respectively, but does comprise the amino acid leucine at amino acid position 330;

(e) the amino acids aspartate, serine, alanine, and valine at amino acid positions 239, 366, 368, and 407, respectively, but does not comprise the amino acids leucine and glutamate at amino acid positions 330 and 332, respectively;

(f) the amino acids leucine, serine, alanine, and valine at amino acid positions 330, 366, 368, and 407, respectively, but does not comprise the amino acids aspartate and glutamate at amino acid positions 239 and 332, respectively; or

(g) the amino acids glutamate, serine, alanine, and valine at amino acid position 332, 366, 368, and 407, respectively, but does not comprise the amino acids aspartate and leucine at amino acid positions 239 and 330, respectively,

wherein the amino acid positions are numbered according to the EU index.

In another aspect, the instant disclosure provides heterodimeric proteins comprising a first and a second Fc polypeptide, wherein: the first Fc polypeptide comprises the amino acids aspartate, leucine, glutamate, and tryptophan at amino acid positions 239, 330, 332, and 366, respectively; and the second Fc polypeptide comprises:

(a) the amino acids serine, alanine, and valine at amino acid positions 366, 368, and 407, respectively, but does not comprise the amino acids aspartate, leucine, and glutamate at amino acid positions 239, 330, and 332, respectively;

(b) the amino acids aspartate, leucine, serine, alanine, and valine at amino acid positions 239, 330, 366, 368, and 407, respectively, but does not comprise the amino acids glutamate at amino acid position 332;

(c) the amino acids leucine, glutamate, serine, alanine, and valine at amino acid positions 330, 332, 366, 368, and 407, respectively, but does not comprise the amino acid aspartate at amino acid position 239;

(d) the amino acids aspartate, glutamate, serine, alanine, and valine at amino acid positions 239, 332, 366, 368, and 407, respectively, but does comprise the amino acid leucine at amino acid position 330;

(e) the amino acids aspartate, serine, alanine, and valine at amino acid positions 239, 366, 368, and 407, respectively, but does not comprise the amino acids leucine and glutamate at amino acid positions 330 and 332, respectively;

(f) the amino acids leucine, serine, alanine, and valine at amino acid positions 330, 366, 368, and 407, respectively, but does not comprise the amino acids aspartate and glutamate at amino acid positions 239 and 332, respectively; or (g) the amino acids glutamate, serine, alanine, and valine at amino acid position 332, 366, 368, and 407, respectively, but does not comprise the amino acids aspartate and leucine at amino acid positions 239 and 330, respectively,

wherein the amino acid positions are numbered according to the EU index.

In another aspect, the instant disclosure provides heterodimeric proteins comprising a irst and a second Fc polypeptide, wherein: the first Fc polypeptide comprises the amino acids aspartate, leucine, and tryptophan at amino acid positions 239, 330, and 366, respectively, but does not comprise the amino acid glutamate at amino acid position 332; and the second Fc polypeptide comprises:

(a) the amino acids serine, alanine, and valine at amino acid positions 366, 368, and 407, respectively, but does not comprise the amino acids aspartate, leucine, and glutamate at amino acid positions 239, 330, and 332, respectively;

(b) the amino acids aspartate, leucine, serine, alanine, and valine at amino acid positions 239, 330, 366, 368, and 407, respectively, but does not comprise the amino acids glutamate at amino acid position 332;

(c) the amino acids leucine, glutamate, serine, alanine, and valine at amino acid positions 330, 332, 366, 368, and 407, respectively, but does not comprise the amino acid aspartate at amino acid position 239;

(d) the amino acids aspartate, glutamate, serine, alanine, and valine at amino acid positions 239, 332, 366, 368, and 407, respectively, but does comprise the amino acid leucine at amino acid position 330;

(e) the amino acids aspartate, serine, alanine, and valine at amino acid positions 239, 366, 368, and 407, respectively, but does not comprise the amino acids leucine and glutamate at amino acid positions 330 and 332, respectively;

(f) the amino acids leucine, serine, alanine, and valine at amino acid positions 330, 366, 368, and 407, respectively, but does not comprise the amino acids aspartate and glutamate at amino acid positions 239 and 332, respectively; or

(g) the amino acids glutamate, serine, alanine, and valine at amino acid position 332, 366, 368, and 407, respectively, but does not comprise the amino acids aspartate and leucine at amino acid positions 239 and 330, respectively,

wherein the amino acid positions are numbered according to the EU index.

In another aspect, the instant disclosure provides heterodimeric proteins comprising a first and a second Fc polypeptide, wherein: the first Fc polypeptide comprises the amino acids leucine, glutamate, and tryptophan at amino acid positions 330, 332, and 366, respectively, but does not comprise the amino acid aspartate at amino acid position 239; and the second Fc polypeptide comprises:

(a) the amino acids serine, alanine, and valine at amino acid positions 366, 368, and 407, respectively, but does not comprise the amino acids aspartate, leucine, and glutamate at amino acid positions 239, 330, and 332, respectively;

(b) the amino acids aspartate, leucine, serine, alanine, and valine at amino acid positions 239, 330, 366, 368, and 407, respectively, but does not comprise the amino acids glutamate at amino acid position 332;

(c) the amino acids leucine, glutamate, serine, alanine, and valine at amino acid positions 330, 332, 366, 368, and 407, respectively, but does not comprise the amino acid aspartate at amino acid position 239;

(d) the amino acids aspartate, glutamate, serine, alanine, and valine at amino acid positions 239, 332, 366, 368, and 407, respectively, but does comprise the amino acid leucine at amino acid position 330;

(e) the amino acids aspartate, serine, alanine, and valine at amino acid positions 239, 366, 368, and 407, respectively, but does not comprise the amino acids leucine and glutamate at amino acid positions 330 and 332, respectively;

(f) the amino acids leucine, serine, alanine, and valine at amino acid positions 330, 366, 368, and 407, respectively, but does not comprise the amino acids aspartate and glutamate at amino acid positions 239 and 332, respectively; or

(g) the amino acids glutamate, serine, alanine, and valine at amino acid position 332, 366, 368, and 407, respectively, but does not comprise the amino acids aspartate and leucine at amino acid positions 239 and 330, respectively,

wherein the amino acid positions are numbered according to the EU index.

In another aspect, the instant disclosure provides heterodimeric proteins comprising a first and a second Fc polypeptide, wherein: the first Fc polypeptide comprises the amino acids aspartate, glutamate, and tryptophan at amino acid positions 239, 332, and 366, respectively, but does not comprise the amino acid leucine at amino acid position 330; and the second Fc polypeptide comprises:

(a) the amino acids serine, alanine, and valine at amino acid positions 366, 368, and 407, respectively, but does not comprise the amino acids aspartate, leucine, and glutamate at amino acid positions 239, 330, and 332, respectively;

(b) the amino acids aspartate, leucine, serine, alanine, and valine at amino acid positions 239, 330, 366, 368, and 407, respectively, but does not comprise the amino acids glutamate at amino acid position 332;

(c) the amino acids leucine, glutamate, serine, alanine, and valine at amino acid positions 330, 332, 366, 368, and 407, respectively, but does not comprise the amino acid aspartate at amino acid position 239;

(d) the amino acids aspartate, glutamate, serine, alanine, and valine at amino acid positions 239, 332, 366, 368, and 407, respectively, but does comprise the amino acid leucine at amino acid position 330;

(e) the amino acids aspartate, serine, alanine, and valine at amino acid positions 239, 366, 368, and 407, respectively, but does not comprise the amino acids leucine and glutamate at amino acid positions 330 and 332, respectively;

(f) the amino acids leucine, serine, alanine, and valine at amino acid positions 330, 366, 368, and 407, respectively, but does not comprise the amino acids aspartate and glutamate at amino acid positions 239 and 332, respectively; or

(g) the amino acids glutamate, serine, alanine, and valine at amino acid position 332, 366, 368, and 407, respectively, but does not comprise the amino acids aspartate and leucine at amino acid positions 239 and 330, respectively,

wherein the amino acid positions are numbered according to the EU index.

In another aspect, the instant disclosure provides heterodimeric proteins comprising a first and a second Fc polypeptide, wherein: the first Fc polypeptide comprises the amino acids aspartate and tryptophan at amino acid positions 239 and 366, respectively, but does not comprise the amino acids leucine and glutamate at amino acid positions 330 and 332, respectively; and the second Fc polypeptide comprises:

(a) the amino acids serine, alanine, and valine at amino acid positions 366, 368, and 407, respectively, but does not comprise the amino acids aspartate, leucine, and glutamate at amino acid positions 239, 330, and 332, respectively;

(b) the amino acids aspartate, leucine, serine, alanine, and valine at amino acid positions 239, 330, 366, 368, and 407, respectively, but does not comprise the amino acids glutamate at amino acid position 332;

(c) the amino acids leucine, glutamate, serine, alanine, and valine at amino acid positions 330, 332, 366, 368, and 407, respectively, but does not comprise the amino acid aspartate at amino acid position 239;

(d) the amino acids aspartate, glutamate, serine, alanine, and valine at amino acid positions 239, 332, 366, 368, and 407, respectively, but does comprise the amino acid leucine at amino acid position 330;

(e) the amino acids aspartate, serine, alanine, and valine at amino acid positions 239, 366, 368, and 407, respectively, but does not comprise the amino acids leucine and glutamate at amino acid positions 330 and 332, respectively;

(f) the amino acids leucine, serine, alanine, and valine at amino acid positions 330, 366, 68, and 407, respectively, but does not comprise the amino acids aspartate and glutamate at amino acid positions 239 and 332, respectively; or

(g) the amino acids glutamate, serine, alanine, and valine at amino acid position 332, 366, 368, and 407, respectively, but does not comprise the amino acids aspartate and leucine at amino acid positions 239 and 330, respectively,

wherein the amino acid positions are numbered according to the EU index.

In another aspect, the instant disclosure provides heterodimeric proteins comprising a first and a second Fc polypeptide, wherein: the first Fc polypeptide comprises the amino acids leucine and tryptophan at amino acid positions 330 and 366, respectively, but does not comprise the amino acids aspartate and glutamate at amino acid positions 239 and 332, respectively; and the second Fc polypeptide comprises:

(a) the amino acids serine, alanine, and valine at amino acid positions 366, 368, and 407, respectively, but does not comprise the amino acids aspartate, leucine, and glutamate at amino acid positions 239, 330, and 332, respectively;

(b) the amino acids aspartate, leucine, serine, alanine, and valine at amino acid positions 239, 330, 366, 368, and 407, respectively, but does not comprise the amino acids glutamate at amino acid position 332;

(c) the amino acids leucine, glutamate, serine, alanine, and valine at amino acid positions 330, 332, 366, 368, and 407, respectively, but does not comprise the amino acid aspartate at amino acid position 239;

(d) the amino acids aspartate, glutamate, serine, alanine, and valine at amino acid positions 239, 332, 366, 368, and 407, respectively, but does comprise the amino acid leucine at amino acid position 330;

(e) the amino acids aspartate, serine, alanine, and valine at amino acid positions 239, 366, 368, and 407, respectively, but does not comprise the amino acids leucine and glutamate at amino acid positions 330 and 332, respectively;

(f) the amino acids leucine, serine, alanine, and valine at amino acid positions 330, 366, 368, and 407, respectively, but does not comprise the amino acids aspartate and glutamate at amino acid positions 239 and 332, respectively; or (g) the amino acids glutamate, serine, alanine, and valine at amino acid position 332, 366, 368, and 407, respectively, but does not comprise the amino acids aspartate and leucine at amino acid positions 239 and 330, respectively,

wherein the amino acid positions are numbered according to the EU index.

In another aspect, the instant disclosure provides heterodimeric proteins comprising a first and a second Fc polypeptide, wherein: the first Fc polypeptide comprises the amino acids glutamate and tryptophan at amino acid positions 332 and 366, respectively, but does not comprise the amino acids aspartate and leucine at amino acid positions 239 and 330, respectively; and the second Fc polypeptide comprises:

(a) the amino acids serine, alanine, and valine at amino acid positions 366, 368, and 407, respectively, but does not comprise the amino acids aspartate, leucine, and glutamate at amino acid positions 239, 330, and 332, respectively;

(b) the amino acids aspartate, leucine, serine, alanine, and valine at amino acid positions 239, 330, 366, 368, and 407, respectively, but does not comprise the amino acids glutamate at amino acid position 332;

(c) the amino acids leucine, glutamate, serine, alanine, and valine at amino acid positions 330, 332, 366, 368, and 407, respectively, but does not comprise the amino acid aspartate at amino acid position 239;

(d) the amino acids aspartate, glutamate, serine, alanine, and valine at amino acid positions 239, 332, 366, 368, and 407, respectively, but does comprise the amino acid leucine at amino acid position 330;

(e) the amino acids aspartate, serine, alanine, and valine at amino acid positions 239, 366, 368, and 407, respectively, but does not comprise the amino acids leucine and glutamate at amino acid positions 330 and 332, respectively;

(f) the amino acids leucine, serine, alanine, and valine at amino acid positions 330, 366, 368, and 407, respectively, but does not comprise the amino acids aspartate and glutamate at amino acid positions 239 and 332, respectively; or

(g) the amino acids glutamate, serine, alanine, and valine at amino acid position 332, 366, 368, and 407, respectively, but does not comprise the amino acids aspartate and leucine at amino acid positions 239 and 330, respectively,

wherein the amino acid positions are numbered according to the EU index.

The Fc polypeptides described herein generally comprise a CH2 domain and a CH3 domain, wherein the C-terminus of the CH2 domain is linked (directly or indirectly) to the N-terminus of the CH3 domain. Any naturally occurring or variant CH2 and/or CH3 domain can be used in the Fc polypeptides described herein. For example, in certain embodiments, the CH2 and/or CH3 domain is a naturally occurring CH2 or CH3 domain from an IgG₁, IgG₂, IgG₃, IgG₄, IgA₁, or IgA₂ antibody heavy chain, e.g., a human IgG₁, IgG₂, IgG₃, IgG₄, IgA₁, or IgA₂ antibody heavy chain. The CH2 and CH3 domains can be from the same or different antibody heavy chains. In certain embodiments, the Fc polypeptide comprises a CH2 and CH3 domain-containing portion from a single antibody heavy chain. In certain embodiments, the CH2 and/or CH3 domain is a variant of a naturally occurring CH2 or CH3 domain, respectively. In certain embodiments, the CH2 and/or CH3 domain is a variant comprising one or more amino acid insertions, deletion, substitutions, or modifications relative to a naturally occurring CH2 or CH3 domain, respectively. In certain embodiments, the CH2 and/or CH3 domain is a chimera of one or more CH2 or CH3 domains, respectively. In certain embodiments, the CH2 domain comprises amino acid positions 231-340 of a naturally occurring hinge region (e.g., human IgG₁), according to the EU index. In certain embodiments, the CH3 domain comprises amino acid positions 341-447 of a naturally occurring hinge region (e.g., human IgG₁), according to the EU index.

In certain embodiments, the Fc polypeptides described herein further comprise a hinge region, wherein the C-terminus of hinge region is linked (directly or indirectly) to the N-terminus of the CH2 domain. For example, in certain embodiments, the hinge region is a naturally occurring hinge region from an IgG₁, IgG₂, IgG₃, IgG₄, IgA₁, or IgA₂ antibody heavy chain, e.g., a human IgG₁, IgG₂, IgG₃, IgG₄, IgA₁, or IgA₂ antibody heavy chain. The hinge region can be from the same or different antibody heavy chain than the CH2 and/or CH3 domains. In certain embodiments, the hinge region is a variant comprising one or more amino acid insertions, deletion, substitutions, or modifications relative to a naturally occurring hinge region. In certain embodiments, the hinge region is a chimera of one or more hinge regions. In certain embodiments, the hinge region comprises amino acid positions 226-229 of a naturally occurring hinge region (e.g., human IgG₁), according to the EU index. In certain embodiments, the hinge region comprises amino acid positions 216-230 of a naturally occurring hinge region (e.g., human IgG₁), according to the EU index. In certain embodiments, the hinge region comprises amino acid positions 216-230 of a naturally occurring hinge region (e.g., human IgG₁), according to the EU index. In certain embodiments, the hinge region is a variant IgG₄ hinge region comprising a serine (S) at amino acid position 228, according to the EU index.

In certain embodiments, the Fc polypeptides described herein further comprise a CH1 domain, wherein the C-terminus of CH1 domain is linked (directly or indirectly) to the N-terminus of the hinge region. For example, in certain embodiments, the CH1 domain is a naturally occurring CH1 domain from an IgG₁, IgG₂, IgG₃, IgG₄, IgA₁, or IgA₂ antibody heavy chain, e.g., a human IgG₁IgG₂, IgG₃, IgG₄, IgA₁, or IgA₂ antibody heavy chain. The CH1 domain can be from the same or different antibody heavy chain than the hinge region, CH2 domain and/or CH3 domain. In certain embodiments, the CH1 domain is a variant comprising one or more amino acid insertions, deletions, substitutions, or modifications relative to a naturally occurring CH1 domain. In certain embodiments, the CH1 domain is a chimera of one or more CH1 domain. In certain embodiments, the CH1 domain comprises amino acid positions 118-215 of a naturally occurring hinge region (e.g., human IgG₁), according to the EU index.

The amino acid sequences of exemplary Fc polypeptides, or portions thereof, are set forth in Table 1 herein.

TABLE 1
Amino acid sequences of exemplary Fc polypeptides or portions thereof

| Description | Amino Acid Sequence | SEQ ID NO |
|---|---|---|
| IgG1m3 | ASTKGPSVFPLAPSSKSTSGGTAALGCLVKDYFPEPVTVS | 1 |
| CH1 | WNSGALTSGVHTFPAVLQSSGLYSLSSVVTVPSSSLGTQT | |
| | YICNVNHKPSNTKVDKRV | |
| Consensus | ASTKGPSVFPLAPSSKSTSGGTAALGCLVKDYFPEPVTVS | 2 |
| CH1 | WNSGALTSGVHTFPAVLQSSGLYSLSSVVTVPSSSLGTQT | |
| | YICNVNHKPSNTKVDKX₁V | |
| | wherein X₁ is R or K | |
| Hinge | EPKSCDKTHTCPPCP | 3 |
| CH2 | APELLGGPSVFLFPPKPKDTLMISRTPEVTCVVVDVSHED | 4 |
| wild-type | PEVKFNWYVDGVEVHNAKTKPREEQYNSTYRVVSVLTVLH | |
| | QDWLNGKEYKCKVSNKALPAPIEKTISKAK | |
| CH2 | APELLGGPDVFLFPPKPKDTLMISRTPEVTCVVVDVSHED | 5 |
| S239D | PEVKFNWYVDGVEVHNAKTKPREEQYNSTYRVVSVLTVLH | |
| | QDWLNGKEYKCKVSNKALPAPIEKTISKAK | |
| CH2 | APELLGGPDVFLFPPKPKDTLMISRTPEVTCVVVDVSHED | 6 |
| S239D/I332E | PEVKFNWYVDGVEVHNAKTKPREEQYNSTYRVVSVLTVLH | |
| | QDWLNGKEYKCKVSNKALPAPEEKTISKAK | |
| CH2 | APELLGGPDVFLFPPKPKDTLMISRTPEVTCVVVDVSHED | 7 |
| S239D/A330L/ | PEVKFNWYVDGVEVHNAKTKPREEQYNSTYRVVSVLTVLH | |
| I332E | QDWLNGKEYKCKVSNKALPLPEEKTISKAK | |
| IgG1m3 | GQPREPQVYTLPPSREEMTKNQVSLTCLVKGFYPSDIAVE | 8 |
| CH3 | WESNGQPENNYKTTPPVLDSDGSFFLYSKLTVDKSRWQQG | |
| | NVFSCSVMHEALHNHYTQKSLSLSPG | |
| Consensus | GQPREPQVYTLPPSRX₁EX₂TKNQVSLTCLVKGFYPSDIAV | 9 |
| CH3 | EWESNGQPENNYKTTPPVLDSDGSFFLYSKLTVDKSRWQQ | |
| | GNVFSCSVMHEX₃LHNHYTQKSLSLSPG | |
| | wherein X₁ is E or D; X₂ is M or L; and | |
| | X₃ is A or G | |
| IgG1m3 | GQPREPQVYTLPPSREEMTKNQVSLWCLVKGFYPSDIAVE | 10 |
| CH3 | WESNGQPENNYKTTPPVLDSDGSFFLYSKLTVDKSRWQQG | |
| T366W | NVFSCSVMHEALHNHYTQKSLSLSPG | |
| Consensus | GQPREPQVYTLPPSRX₁EX₂TKNQVSLWCLVKGFYPSDIAV | 11 |
| CH3 | EWESNGQPENNYKTTPPVLDSDGSFFLYSKLTVDKSRWQQ | |
| T366W | GNVFSCSVMHEX₃LHNHYTQKSLSLSPG | |
| | wherein X₁ is E or D; X₂ is M or L and | |
| | X₃ is A or G | |
| IgG1m3 | GQPREPQVYTLPPSREEMTKNQVSLSCAVKGFYPSDIAVE | 12 |
| CH3 | WESNGQPENNYKTTPPVLDSDGSFFLVSKLTVDKSRWQQG | |
| T366S/L368A/ | NVFSCSVMHEALHNHYTQKSLSLSPG | |
| Y407V | | |
| Consensus | GQPREPQVYTLPPSRX₁EX₂TKNQVSLSCAVKGFYPSDIAV | 13 |
| CH3 | EWESNGQPENNYKTTPPVLDSDGSFFLVSKLTVDKSRWQQ | |
| T366S/L368A/ | GNVFSCSVMHEX₃LHNHYTQKSLSLSPG | |
| Y407V | wherein X₁ is E or D; X₂ is M or L; and | |
| | X₃ is A or G | |
| IgG1m3 | ASTKGPSVFPLAPSSKSTSGGTAALGCLVKDYFPEPVTVS | 14 |
| Constant | WNSGALTSGVHTFPAVLQSSGLYSLSSVVTVPSSSLGTQT | |
| Region | YICNVNHKPSNTKVDKRVEPKSCDKTHTCPPCPAPELLGG | |
| | PSVFLFPPKPKDTLMISRTPEVTCVVVDVSHEDPEVKFNW | |
| | YVDGVEVHNAKTKPREEQYNSTYRVVSVLTVLHQDWLNGK | |
| | EYKCKVSNKALPAPIEKTISKAKGQPREPQVYTLPPSREE | |
| | MTKNQVSLTCLVKGFYPSDIAVEWESNGQPENNYKTTPPV | |
| | LDSDGSFFLYSKLTVDKSRWQQGNVFSCSVMHEALHNHYT | |
| | QKSLSLSPG | |
| Consensus | ASTKGPSVFPLAPSSKSTSGGTAALGCLVKDYFPEPVTVS | 15 |
| Constant | WNSGALTSGVHTFPAVLQSSGLYSLSSVVTVPSSSLGTQT | |
| Region | YICNVNHKPSNTKVDKX₁VEPKSCDKTHTCPPCPAPELLGG | |
| | PSVFLFPPKPKDTLMISRTPEVTCVVVDVSHEDPEVKFNW | |
| | YVDGVEVHNAKTKPREEQYNSTYRVVSVLTVLHQDWLNGK | |
| | EYKCKVSNKALPAPIEKTISKAKGQPREPQVYTLPPSRX₂E | |
| | X₃TKNQVSLTCLVKGFYPSDIAVEWESNGQPENNYKTTPPV | |
| | LDSDGSFFLYSKLTVDKSRWQQGNVFSCSVMHEX₄LHNHYT | |
| | QKSLSLSPG | |
| | wherein X₁ is R or K; X₂ is E or D; X₃ is | |
| | M or L; and X₄ is A or G | |
| IgG1m3 | ASTKGPSVFPLAPSSKSTSGGTAALGCLVKDYFPEPVTVS | 16 |
| Constant | WNSGALTSGVHTFPAVLQSSGLYSLSSVVTVPSSSLGTQT | |
| Region | YICNVNHKPSNTKVDKRVEPKSCDKTHTCPPCPAPELLGG | |
| S239D | PDVFLFPPKPKDTLMISRTPEVTCVVVDVSHEDPEVKFNW | |
| | YVDGVEVHNAKTKPREEQYNSTYRVVSVLTVLHQDWLNGK | |
| | EYKCKVSNKALPAPIEKTISKAKGQPREPQVYTLPPSREE | |
| | MTKNQVSLTCLVKGFYPSDIAVEWESNGQPENNYKTTPPV | |
| | LDSDGSFFLYSKLTVDKSRWQQGNVFSCSVMHEALHNHYT | |
| | QKSLSLSPG | |
| Consensus | ASTKGPSVFPLAPSSKSTSGGTAALGCLVKDYFPEPVTVS | 17 |
| Constant | WNSGALTSGVHTFPAVLQSSGLYSLSSVVTVPSSSLGTQT | |
| Region | YICNVNHKPSNTKVDKX₁VEPKSCDKTHTCPPCPAPELLGG | |
| S239D | PDVFLFPPKPKDTLMISRTPEVTCVVVDVSHEDPEVKFNW | |
| | YVDGVEVHNAKTKPREEQYNSTYRVVSVLTVLHQDWLNGK | |
| | EYKCKVSNKALPAPIEKTISKAKGQPREPQVYTLPPSRX₂E | |
| | X₃TKNQVSLTCLVKGFYPSDIAVEWESNGQPENNYKTTPPV | |
| | LDSDGSFFLYSKLTVDKSRWQQGNVFSCSVMHEX₄LHNHYT | |
| | QKSLSLSPG | |
| | wherein X₁ is R or K; X₂ is E or D; X₃ is | |
| | M or L; and X₄ is A or G | |
| IgG1m3 | ASTKGPSVFPLAPSSKSTSGGTAALGCLVKDYFPEPVTVS | 18 |
| Constant | WNSGALTSGVHTFPAVLQSSGLYSLSSVVTVPSSSLGTQT | |
| Region | YICNVNHKPSNTKVDKRVEPKSCDKTHTCPPCPAPELLGG | |
| S239D/I332E | PDVFLFPPKPKDTLMISRTPEVTCVVVDVSHEDPEVKFNW | |
| | YVDGVEVHNAKTKPREEQYNSTYRVVSVLTVLHQDWLNGK | |
| | EYKCKVSNKALPAPEEKTISKAKGQPREPQVYTLPPSREE | |
| | MTKNQVSLTCLVKGFYPSDIAVEWESNGQPENNYKTTPPV | |
| | LDSDGSFFLYSKLTVDKSRWQQGNVFSCSVMHEALHNHYT | |
| | QKSLSLSPG | |
| Consensus | ASTKGPSVFPLAPSSKSTSGGTAALGCLVKDYFPEPVTVS | 19 |
| Constant | WNSGALTSGVHTFPAVLQSSGLYSLSSVVTVPSSSLGTQT | |
| Region | YICNVNHKPSNTKVDKX₁VEPKSCDKTHTCPPCPAPELLGG | |
| S239D/I332E | PDVFLFPPKPKDTLMISRTPEVTCVVVDVSHEDPEVKFNW | |
| | YVDGVEVHNAKTKPREEQYNSTYRVVSVLTVLHQDWLNGK | |
| | EYKCKVSNKALPAPEEKTISKAKGQPREPQVYTLPPSRX₂E | |
| | X₃TKNQVSLTCLVKGFYPSDIAVEWESNGQPENNYKTTPPV | |
| | LDSDGSFFLYSKLTVDKSRWQQGNVFSCSVMHEX₄LHNHYT | |
| | QKSLSLSPG | |
| | wherein X₁ is R or K; X₂ is E or D; X₃ is | |
| | M or L; and X₄ is A or G | |
| IgG1m3 | ASTKGPSVFPLAPSSKSTSGGTAALGCLVKDYFPEPVTVS | 20 |
| Constant | WNSGALTSGVHTFPAVLQSSGLYSLSSVVTVPSSSLGTQT | |
| Region | YICNVNHKPSNTKVDKRVEPKSCDKTHTCPPCPAPELLGG | |
| S239D/A330L/ | PDVFLFPPKPKDTLMISRTPEVTCVVVDVSHEDPEVKFNW | |
| I332E | YVDGVEVHNAKTKPREEQYNSTYRVVSVLTVLHQDWLNGK | |
| | EYKCKVSNKALPLPEEKTISKAKGQPREPQVYTLPPSREE | |
| | MTKNQVSLTCLVKGFYPSDIAVEWESNGQPENNYKTTPPV | |
| | LDSDGSFFLYSKLTVDKSRWQQGNVFSCSVMHEALHNHYT | |
| | QKSLSLSPG | |
| Consensus | ASTKGPSVFPLAPSSKSTSGGTAALGCLVKDYFPEPVTVS | 21 |
| Constant | WNSGALTSGVHTFPAVLQSSGLYSLSSVVTVPSSSLGTQT | |
| Region | YICNVNHKPSNTKVDKX₁VEPKSCDKTHTCPPCPAPELLGG | |
| S239D/A330L/ | PDVFLFPPKPKDTLMISRTPEVTCVVVDVSHEDPEVKFNW | |
| I332E | YVDGVEVHNAKTKPREEQYNSTYRVVSVLTVLHQDWLNGK | |
| | EYKCKVSNKALPLPEEKTISKAKGQPREPQVYTLPPSRX₂E | |
| | X₃TKNQVSLTCLVKGFYPSDIAVEWESNGQPENNYKTTPPV | |
| | LDSDGSFFLYSKLTVDKSRWQQGNVFSCSVMHEX₄LHNHYT | |
| | QKSLSLSPG | |
| | wherein X₁ is R or K; X₂ is E or D; X₃ is | |
| | M or L; and X₄ is A or G | |
| IgG1m3 | ASTKGPSVFPLAPSSKSTSGGTAALGCLVKDYFPEPVTVS | 22 |
| Constant | WNSGALTSGVHTFPAVLQSSGLYSLSSVVTVPSSSLGTQT | |
| Region | YICNVNHKPSNTKVDKRVEPKSCDKTHTCPPCPAPELLGG | |
| T366W | PSVFLFPPKPKDTLMISRTPEVTCVVVDVSHEDPEVKFNW | |
| | YVDGVEVHNAKTKPREEQYNSTYRVVSVLTVLHQDWLNGK | |
| | EYKCKVSNKALPAPIEKTISKAKGQPREPQVYTLPPSREE | |
| | MTKNQVSLWCLVKGFYPSDIAVEWESNGQPENNYKTTPPV | |
| | LDSDGSFFLYSKLTVDKSRWQQGNVFSCSVMHEALHNHYT | |
| | QKSLSLSPG | |
| Consensus | ASTKGPSVFPLAPSSKSTSGGTAALGCLVKDYFPEPVTVS | 23 |
| Constant | WNSGALTSGVHTFPAVLQSSGLYSLSSVVTVPSSSLGTQT | |
| Region | YICNVNHKPSNTKVDKX₁VEPKSCDKTHTCPPCPAPELLGG | |
| T366W | PSVFLFPPKPKDTLMISRTPEVTCVVVDVSHEDPEVKFNW | |
| | YVDGVEVHNAKTKPREEQYNSTYRVVSVLTVLHQDWLNGK | |
| | EYKCKVSNKALPAPIEKTISKAKGQPREPQVYTLPPSRX₂E | |
| | X₃TKNQVSLWCLVKGFYPSDIAVEWESNGQPENNYKTTPPV | |
| | LDSDGSFFLYSKLTVDKSRWQQGNVFSCSVMHEX₄LHNHYT | |
| | QKSLSLSPG | |
| | wherein X₁ is R or K; X₂ is E or D; X₃ is | |
| | M or L; and X₄ is A or G | |
| IgG1m3 | ASTKGPSVFPLAPSSKSTSGGTAALGCLVKDYFPEPVTVS | 24 |
| Constant | WNSGALTSGVHTFPAVLQSSGLYSLSSVVTVPSSSLGTQT | |
| Region | YICNVNHKPSNTKVDKRVEPKSCDKTHTCPPCPAPELLGG | |
| S239D/A330L/ | PDVFLFPPKPKDTLMISRTPEVTCVVVDVSHEDPEVKFNW | |
| I332E/T366W | YVDGVEVHNAKTKPREEQYNSTYRVVSVLTVLHQDWLNGK | |
| | EYKCKVSNKALPLPEEKTISKAKGQPREPQVYTLPPSREE | |
| | MTKNQVSLWCLVKGFYPSDIAVEWESNGQPENNYKTTPPV | |
| | LDSDGSFFLYSKLTVDKSRWQQGNVFSCSVMHEALHNHYT | |
| | QKSLSLSPG | |
| Consensus | ASTKGPSVFPLAPSSKSTSGGTAALGCLVKDYFPEPVTVS | 25 |
| Constant | WNSGALTSGVHTFPAVLQSSGLYSLSSVVTVPSSSLGTQT | |
| Region | YICNVNHKPSNTKVDKX₁VEPKSCDKTHTCPPCPAPELLGG | |
| S239D/A330L/ | PDVFLFPPKPKDTLMISRTPEVTCVVVDVSHEDPEVKFNW | |
| I332E/T366W | YVDGVEVHNAKTKPREEQYNSTYRVVSVLTVLHQDWLNGK | |
| | EYKCKVSNKALPLPEEKTISKAKGQPREPQVYTLPPSRX₂E | |
| | X₃TKNQVSLWCLVKGFYPSDIAVEWESNGQPENNYKTTPPV | |
| | LDSDGSFFLYSKLTVDKSRWQQGNVFSCSVMHEX₄LHNHYT | |
| | QKSLSLSPG | |
| | wherein X₁ is R or K; X₂ is E or D; X₃ is | |
| | M or L; and X₄ is A or G | |
| IgG1m3 | ASTKGPSVFPLAPSSKSTSGGTAALGCLVKDYFPEPVTVS | 26 |
| Constant | WNSGALTSGVHTFPAVLQSSGLYSLSSVVTVPSSSLGTQT | |
| Region | YICNVNHKPSNTKVDKRVEPKSCDKTHTCPPCPAPELLGG | |
| S239D/I332E/ | PDVFLFPPKPKDTLMISRTPEVTCVVVDVSHEDPEVKFNW | |
| T366W | YVDGVEVHNAKTKPREEQYNSTYRVVSVLTVLHQDWLNGK | |
| | EYKCKVSNKALPAPEEKTISKAKGQPREPQVYTLPPSREE | |
| | MTKNQVSLWCLVKGFYPSDIAVEWESNGQPENNYKTTPPV | |
| | LDSDGSFFLYSKLTVDKSRWQQGNVFSCSVMHEALHNHYT | |
| | QKSLSLSPG | |
| Consensus | ASTKGPSVFPLAPSSKSTSGGTAALGCLVKDYFPEPVTVS | 27 |
| Constant | WNSGALTSGVHTFPAVLQSSGLYSLSSVVTVPSSSLGTQT | |
| Region | YICNVNHKPSNTKVDKX₁VEPKSCDKTHTCPPCPAPELLGG | |
| S239D/I332E/ | PDVFLFPPKPKDTLMISRTPEVTCVVVDVSHEDPEVKFNW | |
| T366W | YVDGVEVHNAKTKPREEQYNSTYRVVSVLTVLHQDWLNGK | |
| | EYKCKVSNKALPAPEEKTISKAKGQPREPQVYTLPPSRX₂E | |
| | X₃TKNQVSLWCLVKGFYPSDIAVEWESNGQPENNYKTTPPV | |
| | LDSDGSFFLYSKLTVDKSRWQQGNVFSCSVMHEX₄LHNHYT | |
| | QKSLSLSPG | |
| | wherein X₁ is R or K; X₂ is E or D; X₃ is | |
| | M or L; and X₄ is A or G | |
| IgG1m3 | ASTKGPSVFPLAPSSKSTSGGTAALGCLVKDYFPEPVTVS | 28 |
| Constant | WNSGALTSGVHTFPAVLQSSGLYSLSSVVTVPSSSLGTQT | |
| Region | YICNVNHKPSNTKVDKRVEPKSCDKTHTCPPCPAPELLGG | |
| S239D/ | PDVFLFPPKPKDTLMISRTPEVTCVVVDVSHEDPEVKFNW | |
| T366W | YVDGVEVHNAKTKPREEQYNSTYRVVSVLTVLHQDWLNGK | |
| | EYKCKVSNKALPAPIEKTISKAKGQPREPQVYTLPPSREE | |
| | MTKNQVSLWCLVKGFYPSDIAVEWESNGQPENNYKTTPPV | |
| | LDSDGSFFLYSKLTVDKSRWQQGNVFSCSVMHEALHNHYT | |
| | QKSLSLSPG | |
| Consensus | ASTKGPSVFPLAPSSKSTSGGTAALGCLVKDYFPEPVTVS | 29 |
| Constant | WNSGALTSGVHTFPAVLQSSGLYSLSSVVTVPSSSLGTQT | |
| Region | YICNVNHKPSNTKVDKX₁VEPKSCDKTHTCPPCPAPELLGG | |
| S239D/ | PDVFLFPPKPKDTLMISRTPEVTCVVVDVSHEDPEVKFNW | |
| T366W | YVDGVEVHNAKTKPREEQYNSTYRVVSVLTVLHQDWLNGK | |
| | EYKCKVSNKALPAPIEKTISKAKGQPREPQVYTLPPSRX₂E | |
| | X₃TKNQVSLWCLVKGFYPSDIAVEWESNGQPENNYKTTPPV | |
| | LDSDGSFFLYSKLTVDKSRWQQGNVFSCSVMHEX₄LHNHYT | |
| | QKSLSLSPG | |
| | wherein X₁ is R or K; X₂ is E or D; X₃ is | |
| | M or L; and X₄ is A or G | |
| IgG1m3 | ASTKGPSVFPLAPSSKSTSGGTAALGCLVKDYFPEPVTVS | 30 |
| Constant | WNSGALTSGVHTFPAVLQSSGLYSLSSVVTVPSSSLGTQT | |
| Region | YICNVNHKPSNTKVDKRVEPKSCDKTHTCPPCPAPELLGG | |
| T366S/L368A | PSVFLFPPKPKDTLMISRTPEVTCVVVDVSHEDPEVKFNW | |
| Y407V | YVDGVEVHNAKTKPREEQYNSTYRVVSVLTVLHQDWLNGK | |
| | EYKCKVSNKALPAPIEKTISKAKGQPREPQVYTLPPSREE | |
| | MTKNQVSLSCAVKGFYPSDIAVEWESNGQPENNYKTTPPV | |
| | LDSDGSFFLVSKLTVDKSRWQQGNVFSCSVMHEALHNHYT | |
| | QKSLSLSPG | |
| Consensus | ASTKGPSVFPLAPSSKSTSGGTAALGCLVKDYFPEPVTVS | 31 |
| Constant | WNSGALTSGVHTFPAVLQSSGLYSLSSVVTVPSSSLGTQT | |
| Region | YICNVNHKPSNTKVDKX₁VEPKSCDKTHTCPPCPAPELLGG | |
| T366S/L368A | PSVFLFPPKPKDTLMISRTPEVTCVVVDVSHEDPEVKFNW | |
| Y407V | YVDGVEVHNAKTKPREEQYNSTYRVVSVLTVLHQDWLNGK | |
| | EYKCKVSNKALPAPIEKTISKAKGQPREPQVYTLPPSRX₂E | |
| | X₃TKNQVSLSCAVKGFYPSDIAVEWESNGQPENNYKTTPPV | |
| | LDSDGSFFLVSKLTVDKSRWQQGNVFSCSVMHEX₄LHNHYT | |
| | QKSLSLSPG | |
| | wherein X₁ is R or K; X₂ is E or D; X₃ is | |
| | M or L; and X₄ is A or G | |
| IgG1m3 | ASTKGPSVFPLAPSSKSTSGGTAALGCLVKDYFPEPVTVS | 32 |
| Constant | WNSGALTSGVHTFPAVLQSSGLYSLSSVVTVPSSSLGTQT | |
| Region | YICNVNHKPSNTKVDKRVEPKSCDKTHTCPPCPAPELLGG | |
| S239D/A330L/ | PDVFLFPPKPKDTLMISRTPEVTCVVVDVSHEDPEVKFNW | |
| I332E/ | YVDGVEVHNAKTKPREEQYNSTYRVVSVLTVLHQDWLNGK | |
| T366S/L368A/ | EYKCKVSNKALPLPEEKTISKAKGQPREPQVYTLPPSREE | |
| Y407V | MTKNQVSLSCAVKGFYPSDIAVEWESNGQPENNYKTTPPV | |
| | LDSDGSFFLVSKLTVDKSRWQQGNVFSCSVMHEALHNHYT | |
| | QKSLSLSPG | |
| Consensus | ASTKGPSVFPLAPSSKSTSGGTAALGCLVKDYFPEPVTVS | 33 |
| Constant | WNSGALTSGVHTFPAVLQSSGLYSLSSVVTVPSSSLGTQT | |
| Region | YICNVNHKPSNTKVDKX₁VEPKSCDKTHTCPPCPAPELLGG | |
| S239D/A330L/ | PDVFLFPPKPKDTLMISRTPEVTCVVVDVSHEDPEVKFNW | |
| I332E/ | YVDGVEVHNAKTKPREEQYNSTYRVVSVLTVLHQDWLNGK | |
| T366S/L368A/ | EYKCKVSNKALPLPEEKTISKAKGQPREPQVYTLPPSRX₂E | |
| Y407V | X₃TKNQVSLSCAVKGFYPSDIAVEWESNGQPENNYKTTPPV | |
| | LDSDGSFFLVSKLTVDKSRWQQGNVFSCSVMHEX₄LHNHYT | |
| | QKSLSLSPG | |
| | wherein X₁ is R or K; X₂ is E or D; X₃ is | |
| | M or L; and X₄ is A or G | |
| IgG1m3 | ASTKGPSVFPLAPSSKSTSGGTAALGCLVKDYFPEPVTVS | 34 |
| Constant | WNSGALTSGVHTFPAVLQSSGLYSLSSVVTVPSSSLGTQT | |
| Region | YICNVNHKPSNTKVDKRVEPKSCDKTHTCPPCPAPELLGG | |
| S239D/I332E/ | PDVFLFPPKPKDTLMISRTPEVTCVVVDVSHEDPEVKFNW | |
| T366S/L368A/ | YVDGVEVHNAKTKPREEQYNSTYRVVSVLTVLHQDWLNGK | |
| Y407V | EYKCKVSNKALPAPEEKTISKAKGQPREPQVYTLPPSREE | |
| | MTKNQVSLSCAVKGFYPSDIAVEWESNGQPENNYKTTPPV | |
| | LDSDGSFFLVSKLTVDKSRWQQGNVFSCSVMHEALHNHYT | |
| | QKSLSLSPG | |
| Consensus | ASTKGPSVFPLAPSSKSTSGGTAALGCLVKDYFPEPVTVS | 35 |
| Constant | WNSGALTSGVHTFPAVLQSSGLYSLSSVVTVPSSSLGTQT | |
| Region | YICNVNHKPSNTKVDKX₁VEPKSCDKTHTCPPCPAPELLGG | |
| S239D/I332E/ | PDVFLFPPKPKDTLMISRTPEVTCVVVDVSHEDPEVKFNW | |
| T366S/L368A/ | YVDGVEVHNAKTKPREEQYNSTYRVVSVLTVLHQDWLNGK | |
| Y407V | EYKCKVSNKALPAPEEKTISKAKGQPREPQVYTLPPSRX₂E | |
| | X₃TKNQVSLSCAVKGFYPSDIAVEWESNGQPENNYKTTPPV | |
| | LDSDGSFFLVSKLTVDKSRWQQGNVFSCSVMHEX₄LHNHYT | |
| | QKSLSLSPG | |
| | wherein X₁ is R or K; X₂ is E or D; X₃ is | |
| | M or L; and X₄ is A or G | |
| IgG1m3 | ASTKGPSVFPLAPSSKSTSGGTAALGCLVKDYFPEPVTVS | 36 |
| Constant | WNSGALTSGVHTFPAVLQSSGLYSLSSVVTVPSSSLGTQT | |
| Region | YICNVNHKPSNTKVDKRVEPKSCDKTHTCPPCPAPELLGG | |
| S239D/ | PDVFLFPPKPKDTLMISRTPEVTCVVVDVSHEDPEVKFNW | |
| T366S/L368A/ | YVDGVEVHNAKTKPREEQYNSTYRVVSVLTVLHQDWLNGK | |
| Y407V | EYKCKVSNKALPAPIEKTISKAKGQPREPQVYTLPPSREE | |
| | MTKNQVSLSCAVKGFYPSDIAVEWESNGQPENNYKTTPPV | |
| | LDSDGSFFLVSKLTVDKSRWQQGNVFSCSVMHEALHNHYT | |
| | QKSLSLSPG | |
| Consensus | ASTKGPSVFPLAPSSKSTSGGTAALGCLVKDYFPEPVTVS | 37 |
| Constant | WNSGALTSGVHTFPAVLQSSGLYSLSSVVTVPSSSLGTQT | |
| Region | YICNVNHKPSNTKVDKX₁VEPKSCDKTHTCPPCPAPELLGG | |
| S239D/ | PDVFLFPPKPKDTLMISRTPEVTCVVVDVSHEDPEVKFNW | |
| T366S/L368A/ | YVDGVEVHNAKTKPREEQYNSTYRVVSVLTVLHQDWLNGK | |
| Y407V | EYKCKVSNKALPAPIEKTISKAKGQPREPQVYTLPPSRX₂E | |
| | X₃TKNQVSLSCAVKGFYPSDIAVEWESNGQPENNYKTTPPV | |
| | LDSDGSFFLVSKLTVDKSRWQQGNVFSCSVMHEX₄LHNHYT | |
| | QKSLSLSPG | |
| | wherein X₁ is R or K; X₂ is E or D; X₃ is | |
| | M or L; and X₄ is A or G | |
| IgG1m3 | APELLGGPSVFLFPPKPKDTLMISRTPEVTCVVVDVSHED | 38 |
| Constant | PEVKFNWYVDGVEVHNAKTKPREEQYNSTYRVVSVLTVLH | |
| Region | QDWLNGKEYKCKVSNKALPAPIEKTISKAKGQPREPQVYT | |
| CH2-CH3 | LPPSREEMTKNQVSLTCLVKGFYPSDIAVEWESNGQPENN | |
| | YKTTPPVLDSDGSFFLYSKLTVDKSRWQQGNVFSCSVMHE | |
| | ALHNHYTQKSLSLSPG | |
| Consensus | APELLGGPSVFLFPPKPKDTLMISRTPEVTCVVVDVSHED | 39 |
| Constant | PEVKFNWYVDGVEVHNAKTKPREEQYNSTYRVVSVLTVLH | |
| Region | QDWLNGKEYKCKVSNKALPAPIEKTISKAKGQPREPQVYT | |
| CH2-CH3 | LPPSRX₁EX₂TKNQVSLTCLVKGFYPSDIAVEWESNGQPEN | |
| | NYKTTPPVLDSDGSFFLYSKLTVDKSRWQQGNVFSCSVMH | |
| | EX₃LHNHYTQKSLSLSPG | |
| | wherein X₁ is E or D; X₂ is M or L; and | |
| | X₃ is A or G | |
| IgG1m3 | APELLGGPDVFLFPPKPKDTLMISRTPEVTCVVVDVSHED | 40 |
| Constant | PEVKFNWYVDGVEVHNAKTKPREEQYNSTYRVVSVLTVLH | |
| Region | QDWLNGKEYKCKVSNKALPAPIEKTISKAKGQPREPQVYT | |
| CH2-CH3 | LPPSREEMTKNQVSLTCLVKGFYPSDIAVEWESNGQPENN | |
| S239D | YKTTPPVLDSDGSFFLYSKLTVDKSRWQQGNVFSCSVMHE | |
| | ALHNHYTQKSLSLSPG | |
| Consensus | APELLGGPDVFLFPPKPKDTLMISRTPEVTCVVVDVSHED | 41 |
| Constant | PEVKFNWYVDGVEVHNAKTKPREEQYNSTYRVVSVLTVLH | |
| Region | QDWLNGKEYKCKVSNKALPAPIEKTISKAKGQPREPQVYT | |
| CH2-CH3 | LPPSRX₁EX₂TKNQVSLTCLVKGFYPSDIAVEWESNGQPEN | |
| S239D | NYKTTPPVLDSDGSFFLYSKLTVDKSRWQQGNVFSCSVMH | |
| | EX₃LHNHYTQKSLSLSPG | |
| | wherein X₁ is E or D; X₂ is M or L; and | |
| | X₃ is A or G | |
| IgG1m3 | APELLGGPDVFLFPPKPKDTLMISRTPEVTCVVVDVSHED | 42 |
| Constant | PEVKFNWYVDGVEVHNAKTKPREEQYNSTYRVVSVLTVLH | |
| Region | QDWLNGKEYKCKVSNKALPAPEEKTISKAKGQPREPQVYT | |
| CH2-CH3 | LPPSREEMTKNQVSLTCLVKGFYPSDIAVEWESNGQPENN | |
| S239D/I332E | YKTTPPVLDSDGSFFLYSKLTVDKSRWQQGNVFSCSVMHE | |
| | ALHNHYTQKSLSLSPG | |
| Consensus | APELLGGPDVFLFPPKPKDTLMISRTPEVTCVVVDVSHED | 43 |
| Constant | PEVKFNWYVDGVEVHNAKTKPREEQYNSTYRVVSVLTVLH | |
| Region | QDWLNGKEYKCKVSNKALPAPEEKTISKAKGQPREPQVYT | |
| CH2-CH3 | LPPSRX₁EX₂TKNQVSLTCLVKGFYPSDIAVEWESNGQPEN | |
| S239D/I332E | NYKTTPPVLDSDGSFFLYSKLTVDKSRWQQGNVFSCSVMH | |
| | EX₃LHNHYTQKSLSLSPG | |
| | wherein X₁ is E or D; X₂ is M or L; and | |
| | X₃ is A or G | |
| IgG1m3 | APELLGGPDVFLFPPKPKDTLMISRTPEVTCVVVDVSHED | 44 |
| Constant | PEVKFNWYVDGVEVHNAKTKPREEQYNSTYRVVSVLTVLH | |
| Region | QDWLNGKEYKCKVSNKALPLPEEKTISKAKGQPREPQVYT | |
| CH2-CH3 | LPPSREEMTKNQVSLTCLVKGFYPSDIAVEWESNGQPENN | |
| S239D/A330L/ | YKTTPPVLDSDGSFFLYSKLTVDKSRWQQGNVFSCSVMHE | |
| I332E | ALHNHYTQKSLSLSPG | |
| Consensus | APELLGGPDVFLFPPKPKDTLMISRTPEVTCVVVDVSHED | 45 |
| Constant | PEVKFNWYVDGVEVHNAKTKPREEQYNSTYRVVSVLTVLH | |
| Region | QDWLNGKEYKCKVSNKALPLPEEKTISKAKGQPREPQVYT | |
| CH2-CH3 | LPPSRX₁EX₂TKNQVSLTCLVKGFYPSDIAVEWESNGQPEN | |
| S239D/A330L/ | NYKTTPPVLDSDGSFFLYSKLTVDKSRWQQGNVFSCSVMH | |
| I332E | EX₃LHNHYTQKSLSLSPG | |
| | wherein X₁ is E or D; X₂ is M or L; and | |
| | X₃ is A or G | |
| IgG1m3 | APELLGGPSVFLFPPKPKDTLMISRTPEVTCVVVDVSHED | 46 |
| Constant | PEVKFNWYVDGVEVHNAKTKPREEQYNSTYRVVSVLTVLH | |
| Region | QDWLNGKEYKCKVSNKALPAPIEKTISKAKGQPREPQVYT | |
| CH2-CH3 | LPPSREEMTKNQVSLWCLVKGFYPSDIAVEWESNGQPENN | |
| T366W | YKTTPPVLDSDGSFFLYSKLTVDKSRWQQGNVFSCSVMHE | |
| | ALHNHYTQKSLSLSPG | |
| Consensus | APELLGGPSVFLFPPKPKDTLMISRTPEVTCVVVDVSHED | 47 |
| Constant | PEVKFNWYVDGVEVHNAKTKPREEQYNSTYRVVSVLTVLH | |
| Region | QDWLNGKEYKCKVSNKALPAPIEKTISKAKGQPREPQVYT | |
| CH2-CH3 | LPPSRX₁EX₂TKNQVSLWCLVKGFYPSDIAVEWESNGQPEN | |
| T366W | NYKTTPPVLDSDGSFFLYSKLTVDKSRWQQGNVFSCSVMH | |
| | EX₃LHNHYTQKSLSLSPG | |
| | wherein X₁ is E or D; X₂ is M or L; and | |
| | X₃ is A or G | |
| IgG1m3 | APELLGGPDVFLFPPKPKDTLMISRTPEVTCVVVDVSHED | 48 |
| Constant | PEVKFNWYVDGVEVHNAKTKPREEQYNSTYRVVSVLTVLH | |
| Region | QDWLNGKEYKCKVSNKALPLPEEKTISKAKGQPREPQVYT | |
| CH2-CH3 | LPPSREEMTKNQVSLWCLVKGFYPSDIAVEWESNGQPENN | |
| S239D/A330L/ | YKTTPPVLDSDGSFFLYSKLTVDKSRWQQGNVFSCSVMHE | |
| I332E/T366W | ALHNHYTQKSLSLSPG | |
| Consensus | APELLGGPDVFLFPPKPKDTLMISRTPEVTCVVVDVSHED | 49 |
| Constant | PEVKFNWYVDGVEVHNAKTKPREEQYNSTYRVVSVLTVLH | |
| Region | QDWLNGKEYKCKVSNKALPLPEEKTISKAKGQPREPQVYT | |
| CH2-CH3 | LPPSRX₁EX₂TKNQVSLWCLVKGFYPSDIAVEWESNGQPEN | |
| S239D/A330L/ | NYKTTPPVLDSDGSFFLYSKLTVDKSRWQQGNVFSCSVMH | |
| I332E/T366W | EX₃LHNHYTQKSLSLSPG | |
| | wherein X₁ is E or D; X₂ is M or L; and | |
| | X₃ is A or G | |
| IgG1m3 | APELLGGPDVFLFPPKPKDTLMISRTPEVTCVVVDVSHED | 50 |
| Constant | PEVKFNWYVDGVEVHNAKTKPREEQYNSTYRVVSVLTVLH | |
| Region | QDWLNGKEYKCKVSNKALPAPEEKTISKAKGQPREPQVYT | |
| CH2-CH3 | LPPSREEMTKNQVSLWCLVKGFYPSDIAVEWESNGQPENN | |
| S239D/I332E/ | YKTTPPVLDSDGSFFLYSKLTVDKSRWQQGNVFSCSVMHE | |
| T366W | ALHNHYTQKSLSLSPG | |
| Consensus | APELLGGPDVFLFPPKPKDTLMISRTPEVTCVVVDVSHED | 51 |
| Constant | PEVKFNWYVDGVEVHNAKTKPREEQYNSTYRVVSVLTVLH | |
| Region | QDWLNGKEYKCKVSNKALPAPEEKTISKAKGQPREPQVYT | |
| CH2-CH3 | LPPSRX₁EX₂TKNQVSLWCLVKGFYPSDIAVEWESNGQPEN | |
| S239D/I332E/ | NYKTTPPVLDSDGSFFLYSKLTVDKSRWQQGNVFSCSVMH | |
| T366W | EX₃LHNHYTQKSLSLSPG | |
| | wherein X₁ is E or D; X₂ is M or L; and | |
| | X₃ is A or G | |
| IgG1m3 | APELLGGPDVFLFPPKPKDTLMISRTPEVTCVVVDVSHED | 52 |
| Constant | PEVKFNWYVDGVEVHNAKTKPREEQYNSTYRVVSVLTVLH | |
| Region | QDWLNGKEYKCKVSNKALPAPIEKTISKAKGQPREPQVYT | |
| CH2-CH3 | LPPSREEMTKNQVSLWCLVKGFYPSDIAVEWESNGQPENN | |
| S239D/ | YKTTPPVLDSDGSFFLYSKLTVDKSRWQQGNVFSCSVMHE | |
| T366W | ALHNHYTQKSLSLSPG | |
| Consensus | APELLGGPDVFLFPPKPKDTLMISRTPEVTCVVVDVSHED | 53 |
| Constant | PEVKFNWYVDGVEVHNAKTKPREEQYNSTYRVVSVLTVLH | |
| Region | QDWLNGKEYKCKVSNKALPAPIEKTISKAKGQPREPQVYT | |
| CH2-CH3 | LPPSRX₁EX₂TKNQVSLWCLVKGFYPSDIAVEWESNGQPEN | |
| S239D/ | NYKTTPPVLDSDGSFFLYSKLTVDKSRWQQGNVFSCSVMH | |
| T366W | EX₃LHNHYTQKSLSLSPG | |
| | wherein X₁ is E or D; X₂ is M or L; and | |
| | X₃ is A or G | |
| IgG1m3 | APELLGGPSVFLFPPKPKDTLMISRTPEVTCVVVDVSHED | 54 |
| Constant | PEVKFNWYVDGVEVHNAKTKPREEQYNSTYRVVSVLTVLH | |
| Region | QDWLNGKEYKCKVSNKALPAPIEKTISKAKGQPREPQVYT | |
| CH2-CH3 | LPPSREEMTKNQVSLSCAVKGFYPSDIAVEWESNGQPENN | |
| T366S/L368A/ | YKTTPPVLDSDGSFFLVSKLTVDKSRWQQGNVFSCSVMHE | |
| Y407V | ALHNHYTQKSLSLSPG | |
| Consensus | APELLGGPSVFLFPPKPKDTLMISRTPEVTCVVVDVSHED | 55 |
| Constant | PEVKFNWYVDGVEVHNAKTKPREEQYNSTYRVVSVLTVLH | |
| Region | QDWLNGKEYKCKVSNKALPAPIEKTISKAKGQPREPQVYT | |
| CH2-CH3 | LPPSRX₁EX₂TKNQVSLSCAVKGFYPSDIAVEWESNGQPEN | |
| T366S/L368A/ | NYKTTPPVLDSDGSFFLVSKLTVDKSRWQQGNVFSCSVMH | |
| Y407V | EX₃LHNHYTQKSLSLSPG | |
| | wherein X₁ is E or D; X₂ is M or L; and | |
| | X₃ is A or G | |
| IgG1m3 | APELLGGPDVFLFPPKPKDTLMISRTPEVTCVVVDVSHED | 56 |
| Constant | PEVKFNWYVDGVEVHNAKTKPREEQYNSTYRVVSVLTVLH | |
| Region | QDWLNGKEYKCKVSNKALPLPEEKTISKAKGQPREPQVYT | |
| CH2-CH3 | LPPSREEMTKNQVSLSCAVKGFYPSDIAVEWESNGQPENN | |
| S239D/A330L/ | YKTTPPVLDSDGSFFLVSKLTVDKSRWQQGNVFSCSVMHE | |
| I332E/ | ALHNHYTQKSLSLSPG | |
| T366S/L368A/ | | |
| Y407V | | |
| Consensus | APELLGGPDVFLFPPKPKDTLMISRTPEVTCVVVDVSHED | 57 |
| Constant | PEVKFNWYVDGVEVHNAKTKPREEQYNSTYRVVSVLTVLH | |
| Region | QDWLNGKEYKCKVSNKALPLPEEKTISKAKGQPREPQVYT | |
| CH2-CH3 | LPPSRX₁EX₂TKNQVSLSCAVKGFYPSDIAVEWESNGQPEN | |
| S239D/A330L/ | NYKTTPPVLDSDGSFFLVSKLTVDKSRWQQGNVFSCSVMH | |
| I332E/ | EX₃LHNHYTQKSLSLSPG | |
| T366S/L368A/ | wherein X₁ is E or D; X₂ is M or L; and | |
| Y407V | X₃ is A or G | |
| IgG1m3 | APELLGGPDVFLFPPKPKDTLMISRTPEVTCVVVDVSHED | 58 |
| Constant | PEVKFNWYVDGVEVHNAKTKPREEQYNSTYRVVSVLTVLH | |
| Region | QDWLNGKEYKCKVSNKALPAPEEKTISKAKGQPREPQVYT | |
| CH2-CH3 | LPPSREEMTKNQVSLSCAVKGFYPSDIAVEWESNGQPENN | |
| S239D/I332E/ | YKTTPPVLDSDGSFFLVSKLTVDKSRWQQGNVFSCSVMHE | |
| T366S/L368A/ | ALHNHYTQKSLSLSPG | |
| Y407V | | |
| Consensus | APELLGGPDVFLFPPKPKDTLMISRTPEVTCVVVDVSHED | 59 |
| Constant | PEVKFNWYVDGVEVHNAKTKPREEQYNSTYRVVSVLTVLH | |
| Region | QDWLNGKEYKCKVSNKALPAPEEKTISKAKGQPREPQVYT | |
| CH2-CH3 | LPPSRX₁EX₂TKNQVSLSCAVKGFYPSDIAVEWESNGQPEN | |
| S239D/I332E/ | NYKTTPPVLDSDGSFFLVSKLTVDKSRWQQGNVFSCSVMH | |
| T366S/L368A/ | EX₃LHNHYTQKSLSLSPG | |
| Y407V | wherein X₁ is E or D; X₂ is M or L; and | |
| | X₃ is A or G | |
| IgG1m3 | APELLGGPDVFLFPPKPKDTLMISRTPEVTCVVVDVSHED | 60 |
| Constant | PEVKFNWYVDGVEVHNAKTKPREEQYNSTYRVVSVLTVLH | |
| Region | QDWLNGKEYKCKVSNKALPAPIEKTISKAKGQPREPQVYT | |
| CH2-CH3 | LPPSREEMTKNQVSLSCAVKGFYPSDIAVEWESNGQPENN | |
| S239D/ | YKTTPPVLDSDGSFFLVSKLTVDKSRWQQGNVFSCSVMHE | |
| T366S/L368A/ | ALHNHYTQKSLSLSPG | |
| Y407V | | |
| Consensus | APELLGGPDVFLFPPKPKDTLMISRTPEVTCVVVDVSHED | 61 |
| Constant | PEVKFNWYVDGVEVHNAKTKPREEQYNSTYRVVSVLTVLH | |
| Region | QDWLNGKEYKCKVSNKALPAPIEKTISKAKGQPREPQVYT | |
| CH2-CH3 | LPPSRX₁EX₂TKNQVSLSCAVKGFYPSDIAVEWESNGQPEN | |
| S239D/ | NYKTTPPVLDSDGSFFLVSKLTVDKSRWQQGNVFSCSVMH | |
| T366S/L368A/ | EX₃LHNHYTQKSLSLSPG | |
| Y407V | wherein X₁ is E or D; X₂ is M or L; and | |
| | X₃ is A or G | |
| IgG1m3 | CPPCPAPELLGGPDVFLFPPKPKDTLMISRTPEVTCVVVD | 62 |
| Fc Region | VSHEDPEVKFNWYVDGVEVHNAKTKPREEQYNSTYRVVSV | |
| S239D/A330L/ | LTVLHQDWLNGKEYKCKVSNKALPLPEEKTISKAKGQPRE | |
| I332E/T366W | PQVYTLPPSREEMTKNQVSLWCLVKGFYPSDIAVEWESNG | |
| | QPENNYKTTPPVLDSDGSFFLYSKLTVDKSRWQQGNVFSC | |
| | SVMHEALHNHYTQKSLSLSPG | |
| Consensus | CPPCPAPELLGGPDVFLFPPKPKDTLMISRTPEVTCVVVD | 63 |
| Fc Region | VSHEDPEVKFNWYVDGVEVHNAKTKPREEQYNSTYRVVSV | |
| S239D/A330L/ | LTVLHQDWLNGKEYKCKVSNKALPLPEEKTISKAKGQPRE | |
| I332E/T366W | PQVYTLPPSRX₁EX₂TKNQVSLWCLVKGFYPSDIAVEWESN | |
| | GQPENNYKTTPPVLDSDGSFFLYSKLTVDKSRWQQGNVFS | |
| | CSVMHEX₃LHNHYTQKSLSLSPG | |
| | wherein X₁ is E or D; X₂ is M or L; and | |
| | X₃ is A or G | |
| IgG1m3 | CPPCPAPELLGGPDVFLFPPKPKDTLMISRTPEVTCVVVD | 64 |
| Fc Region | VSHEDPEVKFNWYVDGVEVHNAKTKPREEQYNSTYRVVSV | |
| S239D/I332E/ | LTVLHQDWLNGKEYKCKVSNKALPAPEEKTISKAKGQPRE | |
| T366W | PQVYTLPPSREEMTKNQVSLWCLVKGFYPSDIAVEWESNG | |
| | QPENNYKTTPPVLDSDGSFFLYSKLTVDKSRWQQGNVFSC | |
| | SVMHEALHNHYTQKSLSLSPG | |
| Consensus | CPPCPAPELLGGPDVFLFPPKPKDTLMISRTPEVTCVVVD | 65 |
| Fe Region | VSHEDPEVKFNWYVDGVEVHNAKTKPREEQYNSTYRVVSV | |
| S239D/I332E/ | LTVLHQDWLNGKEYKCKVSNKALPAPEEKTISKAKGQPRE | |
| T366W | PQVYTLPPSRX₁EX₂TKNQVSLWCLVKGFYPSDIAVEWESN | |
| | GQPENNYKTTPPVLDSDGSFFLYSKLTVDKSRWQQGNVFS | |
| | CSVMHEX₃LHNHYTQKSLSLSPG | |
| | wherein X₁ is E or D; X₂ is M or L; and | |
| | X₃ is A or G | |
| IgG1m3 | CPPCPAPELLGGPDVFLFPPKPKDTLMISRTPEVTCVVVD | 66 |
| Fe Region | VSHEDPEVKFNWYVDGVEVHNAKTKPREEQYNSTYRVVSV | |
| S239D/ | LTVLHQDWLNGKEYKCKVSNKALPAPIEKTISKAKGQPRE | |
| T366S/L368A/ | PQVYTLPPSREEMTKNQVSLSCAVKGFYPSDIAVEWESNG | |
| Y407V | QPENNYKTTPPVLDSDGSFFLVSKLTVDKSRWQQGNVFSC | |
| | SVMHEALHNHYTQKSLSLSPG | |
| Consensus | CPPCPAPELLGGPDVFLFPPKPKDTLMISRTPEVTCVVVD | 67 |
| Fe Region | VSHEDPEVKFNWYVDGVEVHNAKTKPREEQYNSTYRVVSV | |
| S239D/ | LTVLHQDWLNGKEYKCKVSNKALPAPIEKTISKAKGQPRE | |
| T366S/L368A/ | PQVYTLPPSRX₁EX₂TKNQVSLSCAVKGFYPSDIAVEWESN | |
| Y407V | GQPENNYKTTPPVLDSDGSFFLVSKLTVDKSRWQQGNVFS | |
| | CSVMHEX₃LHNHYTQKSLSLSPG | |
| | wherein X₁ is E or D; X₂ is M or L; and | |
| | X₃ is A or G | |
| IgG1m3 | ASTKGPSVFPLAPSSKSTSGGTAALGCLVKDYFPEPVTVS | 68 |
| Constant | WNSGALTSGVHTFPAVLQSSGLYSLSSVVTVPSSSLGTQT | |
| Region | YICNVNHKPSNTKVDKRVEPKSCDKTHTCPPCPAPELLGG | |
| S239D/A330L/ | PDVFLFPPKPKDTLMISRTPEVTCVVVDVSHEDPEVKFNW | |
| T366W | YVDGVEVHNAKTKPREEQYNSTYRVVSVLTVLHQDWLNGK | |
| | EYKCKVSNKALPLPIEKTISKAKGQPREPQVYTLPPSREE | |
| | MTKNQVSLWCLVKGFYPSDIAVEWESNGQPENNYKTTPPV | |
| | LDSDGSFFLYSKLTVDKSRWQQGNVFSCSVMHEALHNHYT | |
| | QKSLSLSPG | |
| IgG1m3 | ASTKGPSVFPLAPSSKSTSGGTAALGCLVKDYFPEPVTVS | 69 |
| Constant | WNSGALTSGVHTFPAVLQSSGLYSLSSVVTVPSSSLGTQT | |
| Region | YICNVNHKPSNTKVDKRVEPKSCDKTHTCPPCPAPELLGG | |
| A330L/ | PSVFLFPPKPKDTLMISRTPEVTCVVVDVSHEDPEVKFNW | |
| T366W | YVDGVEVHNAKTKPREEQYNSTYRVVSVLTVLHQDWLNGK | |
| | EYKCKVSNKALPLPIEKTISKAKGQPREPQVYTLPPSREE | |
| | MTKNQVSLWCLVKGFYPSDIAVEWESNGQPENNYKTTPPV | |
| | LDSDGSFFLYSKLTVDKSRWQQGNVFSCSVMHEALHNHYT | |
| | QKSLSLSPG | |
| IgG1m3 | ASTKGPSVFPLAPSSKSTSGGTAALGCLVKDYFPEPVTVS | 70 |
| Constant | WNSGALTSGVHTFPAVLQSSGLYSLSSVVTVPSSSLGTQT | |
| Region | YICNVNHKPSNTKVDKRVEPKSCDKTHTCPPCPAPELLGG | |
| I332E/ | PSVFLFPPKPKDTLMISRTPEVTCVVVDVSHEDPEVKFNW | |
| T366W | YVDGVEVHNAKTKPREEQYNSTYRVVSVLTVLHQDWLNGK | |
| | EYKCKVSNKALPAPEEKTISKAKGQPREPQVYTLPPSREE | |
| | MTKNQVSLWCLVKGFYPSDIAVEWESNGQPENNYKTTPPV | |
| | LDSDGSFFLYSKLTVDKSRWQQGNVFSCSVMHEALHNHYT | |
| | QKSLSLSPG | |
| IgG1m3 | ASTKGPSVFPLAPSSKSTSGGTAALGCLVKDYFPEPVTVS | 71 |
| Constant | WNSGALTSGVHTFPAVLQSSGLYSLSSVVTVPSSSLGTQT | |
| Region | YICNVNHKPSNTKVDKRVEPKSCDKTHTCPPCPAPELLGG | |
| S239D/A330L/ | PDVFLFPPKPKDTLMISRTPEVTCVVVDVSHEDPEVKFNW | |
| T366S/L368A/ | YVDGVEVHNAKTKPREEQYNSTYRVVSVLTVLHQDWLNGK | |
| Y407V | EYKCKVSNKALPLPIEKTISKAKGQPREPQVYTLPPSREE | |
| | MTKNQVSLSCAVKGFYPSDIAVEWESNGQPENNYKTTPPV | |
| | LDSDGSFFLVSKLTVDKSRWQQGNVFSCSVMHEALHNHYT | |
| | QKSLSLSPG | |
| IgG1m3 | ASTKGPSVFPLAPSSKSTSGGTAALGCLVKDYFPEPVTVS | 72 |
| Constant | WNSGALTSGVHTFPAVLQSSGLYSLSSVVTVPSSSLGTQT | |
| Region | YICNVNHKPSNTKVDKRVEPKSCDKTHTCPPCPAPELLGG | |
| A330L/I332E/ | PSVFLFPPKPKDTLMISRTPEVTCVVVDVSHEDPEVKFNW | |
| T366S/L368A/ | YVDGVEVHNAKTKPREEQYNSTYRVVSVLTVLHQDWLNGK | |
| Y407V | EYKCKVSNKALPLPEEKTISKAKGQPREPQVYTLPPSREE | |
| | MTKNQVSLSCAVKGFYPSDIAVEWESNGQPENNYKTTPPV | |
| | LDSDGSFFLVSKLTVDKSRWQQGNVFSCSVMHEALHNHYT | |
| | QKSLSLSPG | |
| IgG1m3 | ASTKGPSVFPLAPSSKSTSGGTAALGCLVKDYFPEPVTVS | 73 |
| Constant | WNSGALTSGVHTFPAVLQSSGLYSLSSVVTVPSSSLGTQT | |
| Region | YICNVNHKPSNTKVDKRVEPKSCDKTHTCPPCPAPELLGG | |
| A330L/ | PSVFLFPPKPKDTLMISRTPEVTCVVVDVSHEDPEVKFNW | |
| T366S/L368A/ | YVDGVEVHNAKTKPREEQYNSTYRVVSVLTVLHQDWLNGK | |
| Y407V | EYKCKVSNKALPLPIEKTISKAKGQPREPQVYTLPPSREE | |
| | MTKNQVSLSCAVKGFYPSDIAVEWESNGQPENNYKTTPPV | |
| | LDSDGSFFLVSKLTVDKSRWQQGNVFSCSVMHEALHNHYT | |
| | QKSLSLSPG | |
| IgG1m3 | ASTKGPSVFPLAPSSKSTSGGTAALGCLVKDYFPEPVTVS | 74 |
| Constant | WNSGALTSGVHTFPAVLQSSGLYSLSSVVTVPSSSLGTQT | |
| Region | YICNVNHKPSNTKVDKRVEPKSCDKTHTCPPCPAPELLGG | |
| I332E/ | PSVFLFPPKPKDTLMISRTPEVTCVVVDVSHEDPEVKFNW | |
| T366S/L368A/ | YVDGVEVHNAKTKPREEQYNSTYRVVSVLTVLHQDWLNGK | |
| Y407V | EYKCKVSNKALPAPEEKTISKAKGQPREPQVYTLPPSREE | |
| | MTKNQVSLSCAVKGFYPSDIAVEWESNGQPENNYKTTPPV | |
| | LDSDGSFFLVSKLTVDKSRWQQGNVFSCSVMHEALHNHYT | |
| | QKSLSLSPG | |
| IgG1m3 | ASTKGPSVFPLAPSSKSTSGGTAALGCLVKDYFPEPVTVS | 75 |
| Constant | WNSGALTSGVHTFPAVLQSSGLYSLSSVVTVPSSSLGTQT | |
| Region | YICNVNHKPSNTKVDKRVEPKSCDKTHTCPPCPAPELLGG | |
| A330L/I332E/ | PSVFLFPPKPKDTLMISRTPEVTCVVVDVSHEDPEVKFNW | |
| T366W | YVDGVEVHNAKTKPREEQYNSTYRVVSVLTVLHQDWLNGK | |
| | EYKCKVSNKALPLPEEKTISKAKGQPREPQVYTLPPSREE | |
| | MTKNQVSLWCLVKGFYPSDIAVEWESNGQPENNYKTTPPV | |
| | LDSDGSFFLYSKLTVDKSRWQQGNVFSCSVMHEALHNHYT | |
| | QKSLSLSPG | |
| IgG1m3 | CPPCPAPELLGGPDVFLFPPKPKDTLMISRTPEVTCVVVD | 76 |
| Fc Region | VSHEDPEVKFNWYVDGVEVHNAKTKPREEQYNSTYRVVSV | |
| S239D/A330L/ | LTVLHQDWLNGKEYKCKVSNKALPLPIEKTISKAKGQPRE | |
| T366W | PQVYTLPPSREEMTKNQVSLWCLVKGFYPSDIAVEWESNG | |
| | QPENNYKTTPPVLDSDGSFFLYSKLTVDKSRWQQGNVFSC | |
| | SVMHEALHNHYTQKSLSLSPG | |
| IgG1m3 | CPPCPAPELLGGPSVFLFPPKPKDTLMISRTPEVTCVVVD | 77 |
| Fc Region | VSHEDPEVKFNWYVDGVEVHNAKTKPREEQYNSTYRVVSV | |
| A330L/ | LTVLHQDWLNGKEYKCKVSNKALPLPIEKTISKAKGQPRE | |
| T366W | PQVYTLPPSREEMTKNQVSLWCLVKGFYPSDIAVEWESNG | |
| | QPENNYKTTPPVLDSDGSFFLYSKLTVDKSRWQQGNVFSC | |
| | SVMHEALHNHYTQKSLSLSPG | |
| IgG1m3 | CPPCPAPELLGGPSVFLFPPKPKDTLMISRTPEVTCVVVD | 78 |
| Fc Region | VSHEDPEVKFNWYVDGVEVHNAKTKPREEQYNSTYRVVSV | |
| I332E/ | LTVLHQDWLNGKEYKCKVSNKALPAPEEKTISKAKGQPRE | |
| T366W | PQVYTLPPSREEMTKNQVSLWCLVKGFYPSDIAVEWESNG | |
| | QPENNYKTTPPVLDSDGSFFLYSKLTVDKSRWQQGNVFSC | |
| | SVMHEALHNHYTQKSLSLSPG | |
| IgG1m3 | CPPCPAPELLGGPDVFLFPPKPKDTLMISRTPEVTCVVVD | 79 |
| Fc Region | VSHEDPEVKFNWYVDGVEVHNAKTKPREEQYNSTYRVVSV | |
| S239D/A330L/ | LTVLHQDWLNGKEYKCKVSNKALPLPIEKTISKAKGQPRE | |
| T366S/L368A/ | PQVYTLPPSREEMTKNQVSLSCAVKGFYPSDIAVEWESNG | |
| Y407V | QPENNYKTTPPVLDSDGSFFLVSKLTVDKSRWQQGNVFSC | |
| | SVMHEALHNHYTQKSLSLSPG | |
| IgG1m3 | CPPCPAPELLGGPSVFLFPPKPKDTLMISRTPEVTCVVVD | 80 |
| Fc Region | VSHEDPEVKFNWYVDGVEVHNAKTKPREEQYNSTYRVVSV | |
| A330L/I332E/ | LTVLHQDWLNGKEYKCKVSNKALPLPEEKTISKAKGQPRE | |
| T366S/L368A/ | PQVYTLPPSREEMTKNQVSLSCAVKGFYPSDIAVEWESNG | |
| Y407V | QPENNYKTTPPVLDSDGSFFLVSKLTVDKSRWQQGNVFSC | |
| | SVMHEALHNHYTQKSLSLSPG | |
| IgG1m3 | CPPCPAPELLGGPSVFLFPPKPKDTLMISRTPEVTCVVVD | 81 |
| Fc Region | VSHEDPEVKFNWYVDGVEVHNAKTKPREEQYNSTYRVVSV | |
| A330L/ | LTVLHQDWLNGKEYKCKVSNKALPLPIEKTISKAKGQPRE | |
| T366S/L368A/ | PQVYTLPPSREEMTKNQVSLSCAVKGFYPSDIAVEWESNG | |
| Y407V | QPENNYKTTPPVLDSDGSFFLVSKLTVDKSRWQQGNVFSC | |
| | SVMHEALHNHYTQKSLSLSPG | |
| IgG1m3 | CPPCPAPELLGGPSVFLFPPKPKDTLMISRTPEVTCVVVD | 82 |
| Fc Region | VSHEDPEVKFNWYVDGVEVHNAKTKPREEQYNSTYRVVSV | |
| I332E/ | LTVLHQDWLNGKEYKCKVSNKALPAPEEKTISKAKGQPRE | |
| T366S/L368A/ | PQVYTLPPSREEMTKNQVSLSCAVKGFYPSDIAVEWESNG | |
| Y407V | QPENNYKTTPPVLDSDGSFFLVSKLTVDKSRWQQGNVFSC | |
| | SVMHEALHNHYTQKSLSLSPG | |
| IgG1m3 | CPPCPAPELLGGPSVFLFPPKPKDTLMISRTPEVTCVVVD | 83 |
| Fc Region | VSHEDPEVKFNWYVDGVEVHNAKTKPREEQYNSTYRVVSV | |
| A330L/I332E/ | LTVLHQDWLNGKEYKCKVSNKALPLPEEKTISKAKGQPRE | |
| T366W | PQVYTLPPSREEMTKNQVSLWCLVKGFYPSDIAVEWESNG | |
| | QPENNYKTTPPVLDSDGSFFLYSKLTVDKSRWQQGNVFSC | |
| | SVMHEALHNHYTQKSLSLSPG | |
| IgG1m3 | CPPCPAPELLGGPSVFLFPPKPKDTLMISRTPEVTCVVVD | 84 |
| Fc Region | VSHEDPEVKFNWYVDGVEVHNAKTKPREEQYNSTYRVVSV | |
| T366W | LTVLHQDWLNGKEYKCKVSNKALPAPIEKTISKAKGQPRE | |
| | PQVYTLPPSREEMTKNQVSLWCLVKGFYPSDIAVEWESNG | |
| | QPENNYKTTPPVLDSDGSFFLYSKLTVDKSRWQQGNVFSC | |
| | SVMHEALHNHYTQKSLSLSPG | |
| IgG1m3 | CPPCPAPELLGGPDVFLFPPKPKDTLMISRTPEVTCVVVD | 85 |
| Fc Region | VSHEDPEVKFNWYVDGVEVHNAKTKPREEQYNSTYRVVSV | |
| S239D/ | LTVLHQDWLNGKEYKCKVSNKALPAPIEKTISKAKGQPRE | |
| T366W | PQVYTLPPSREEMTKNQVSLWCLVKGFYPSDIAVEWESNG | |
| | QPENNYKTTPPVLDSDGSFFLYSKLTVDKSRWQQGNVFSC | |
| | SVMHEALHNHYTQKSLSLSPG | |
| IgG1m3 | CPPCPAPELLGGPSVFLFPPKPKDTLMISRTPEVTCVVVD | 86 |
| Fc Region | VSHEDPEVKFNWYVDGVEVHNAKTKPREEQYNSTYRVVSV | |
| T366S/L368A/ | LTVLHQDWLNGKEYKCKVSNKALPAPIEKTISKAKGQPRE | |
| Y407V | PQVYTLPPSREEMTKNQVSLSCAVKGFYPSDIAVEWESNG | |
| | QPENNYKTTPPVLDSDGSFFLVSKLTVDKSRWQQGNVFSC | |
| | SVMHEALHNHYTQKSLSLSPG | |
| IgG1m3 | CPPCPAPELLGGPDVFLFPPKPKDTLMISRTPEVTCVVVD | 87 |
| Fc Region | VSHEDPEVKFNWYVDGVEVHNAKTKPREEQYNSTYRVVSV | |
| S239D/I332E/ | LTVLHQDWLNGKEYKCKVSNKALPAPEEKTISKAKGQPRE | |
| T366S/L368A/ | PQVYTLPPSREEMTKNQVSLSCAVKGFYPSDIAVEWESNG | |
| Y407V | QPENNYKTTPPVLDSDGSFFLVSKLTVDKSRWQQGNVFSC | |
| | SVMHEALHNHYTQKSLSLSPG | |

In certain embodiments, the heterodimeric protein comprises: a first Fc polypeptide comprising an amino acid sequence that is at least 75% identical (e.g., at least 75, 76, 77, 78, 79, 80, 81, 82, 83, 84, 85, 86, 87, 88, 89, 90, 91, 92, 93, 94, 95, 96, 97, 98, 99, or 100% identical) to the amino acid sequence of SEQ ID NO: 24, 25, 26, 27, 48, 49, 50, 51, 62, 63, 64, or 65; and/or a second Fc polypeptide comprising an amino acid sequence that is at least 75% identical (e.g., at least 75, 76, 77, 78, 79, 80, 81, 82, 83, 84, 85, 86, 87, 88, 89, 90, 91, 92, 93, 94, 95, 96, 97, 98, 99, or 100% identical) to the amino acid sequence of SEQ ID NO: 36, 37, 60, 61, 66 or 67. In certain embodiments, the first Fc polynucleotide comprises the amino acid sequence of SEQ ID NO: 24, 25, 26, 27, 48, 49, 50, 51, 62, 63, 64, or 65; and/or the second Fc polynucleotide comprises the amino acid sequence of SEQ ID NO: 36, 37, 60, 61, 66 or 67. Exemplary pairs of amino acid sequences that can be incorporated into the first and second Fc polypeptides of heterodimeric proteins are set forth in Table 2 herein. In certain, embodiments, the first Fc polypeptide and the second Fc polypeptide, respectively, comprise the amino acid sequences set forth in the first and second column of any row of Table 2.

TABLE 2
Exemplary pairs of amino acid sequences that can be
incorporated into first and second Fc polypeptides

| SEQ ID NO of first | SEQ ID NO of second |
|---|---|
| Fc polypeptide sequence | Fc polypeptide sequence |
| 24 | 36 |
| 24 | 37 |
| 25 | 36 |
| 25 | 37 |
| 26 | 36 |
| 26 | 37 |
| 27 | 36 |
| 27 | 37 |
| 48 | 60 |
| 48 | 61 |
| 49 | 60 |
| 49 | 61 |
| 50 | 60 |
| 50 | 61 |
| 51 | 60 |
| 51 | 61 |
| 62 | 66 |
| 62 | 67 |
| 63 | 66 |
| 63 | 67 |
| 64 | 66 |
| 64 | 67 |
| 65 | 66 |
| 65 | 67 |

In certain embodiments, the first Fc polypeptide and second Fc polypeptide comprise amino acid sequences that are at least 75% identical (e.g., at least 75, 76, 77, 78, 79, 80, 81, 82, 83, 84, 85, 86, 87, 88, 89, 90, 91, 92, 93, 94, 95, 96, 97, 98, 99, or 100% identical) to the amino acid sequence of SEQ ID NOs: 24 and 36; 24 and 37; 25 and 36; 25 and 37; 26 and 36; 26 and 37; 27 and 36; 27 and 37; 48 and 60; 48 and 61; 49 and 60; 49 and 61; 50 and 60; 50 and 61; 51 and 60; 51 and 61; 62 and 66; 62 and 67; 63 and 66; 63 and 67; 64 and 66; 64 and 67; 65 and 66; or 65 and 67, respectively. In certain embodiments, the first Fc polypeptide and second Fc polypeptide comprise the amino acid sequences of SEQ ID NOs: 24 and 36; 24 and 37; 25 and 36; 25 and 37; 26 and 36; 26 and 37; 27 and 36; 27 and 37; 48 and 60; 48 and 61; 49 and 60; 49 and 61; 50 and 60; 50 and 61; 51 and 60; 51 and 61; 62 and 66; 62 and 67; 63 and 66; 63 and 67; 64 and 66; 64 and 67; 65 and 66; or 65 and 67, respectively.

In an embodiment, the first Fc polypeptide comprises the amino acid sequence of SEQ ID NO: 22 and the second Fc polypeptide comprises the amino acid sequence of SEQ ID NO: 30, 34, 36, 71, 72, 73, or 74. In another embodiment, the first Fc polypeptide comprises the amino acid sequence of SEQ ID NO: 24 and the second Fc polypeptide comprises the amino acid sequence of SEQ ID NO: 30, 34, 36, 71, 72, 73, or 74. In another embodiment, the first Fc polypeptide comprises the amino acid sequence of SEQ ID NO: 68 and the second Fc polypeptide comprises the amino acid sequence of SEQ ID NO: 30, 34, 36, 71, 72, 73, or 74. In another embodiment, the first Fc polypeptide comprises the amino acid sequence of SEQ ID NO: 26 and the second Fc polypeptide comprises the amino acid sequence of SEQ ID NO: 30, 34, 36, 71, 72, 73, or 74. In another embodiment, the first Fc polypeptide comprises the amino acid sequence of SEQ ID NO: 75 and the second Fc polypeptide comprises the amino acid sequence of SEQ ID NO: 30, 34, 36, 71, 72, 73, or 74. In another embodiment, the first Fc polypeptide comprises the amino acid sequence of SEQ ID NO: 28 and the second Fc polypeptide comprises the amino acid sequence of SEQ ID NO: 30, 34, 36, 71, 72, 73, or 74. In another embodiment, the first Fc polypeptide comprises the amino acid sequence of SEQ ID NO: 69 and the second Fc polypeptide comprises the amino acid sequence of SEQ ID NO: 30, 34, 36, 71, 72, 73, or 74. In another embodiment, the first Fc polypeptide comprises the amino acid sequence of SEQ ID NO: 70 and the second Fc polypeptide comprises the amino acid sequence of SEQ ID NO: 30, 34, 36, 71, 72, 73, or 74.

In an embodiment, the first Fc polypeptide comprises the amino acid sequence of SEQ ID NO: 84 and the second Fc polypeptide comprises the amino acid sequence of SEQ ID NO: 66, 79, 80, 81, 82, 86, or 87. In another embodiment, the first Fc polypeptide comprises the amino acid sequence of SEQ ID NO: 62 and the second Fc polypeptide comprises the amino acid sequence of SEQ ID NO: 66, 79, 80, 81, 82, 86, or 87. In another embodiment, the first Fc polypeptide comprises the amino acid sequence of SEQ ID NO: 76 and the second Fc polypeptide comprises the amino acid sequence of SEQ ID NO: 66, 79, 80, 81, 82, 86, or 87. In another embodiment, the first Fc polypeptide comprises the amino acid sequence of SEQ ID NO: 64 and the second Fc polypeptide comprises the amino acid sequence of SEQ ID NO: 66, 79, 80, 81, 82, 86, or 87. In another embodiment, the first Fc polypeptide comprises the amino acid sequence of SEQ ID NO: 83 and the second Fc polypeptide comprises the amino acid sequence of SEQ ID NO: 66, 79, 80, 81, 82, 86, or 87. In another embodiment, the first Fc polypeptide comprises the amino acid sequence of SEQ ID NO: 85 and the second Fc polypeptide comprises the amino acid sequence of SEQ ID NO: 66, 79, 80, 81, 82, 86, or 87. In another embodiment, the first Fc polypeptide comprises the amino acid sequence of SEQ ID NO: 77 and the second Fc polypeptide comprises the amino acid sequence of SEQ ID NO: 66, 79, 80, 81, 82, 86, or 87. In another embodiment, the first Fc polypeptide comprises the amino acid sequence of SEQ ID NO: 78 and the second Fc polypeptide comprises the amino acid sequence of SEQ ID NO: 66, 79, 80, 81, 82, 86, or 87.

In certain embodiments, the first and/or the second Fc polypeptide further comprises one or more antigen-binding moieties. An antigen-binding moiety can be linked (directly or indirectly) to any portion of an Fc polypeptide. For example, antigen-binding moieties can be linked (directly or indirectly) to the N and/or C terminus of an Fc polypeptide. Additionally or alternatively, antigen-binding moieties can be linked together in tandem in an Fc polypeptide.

In certain embodiments, the first and the second Fc polypeptides in a heterodimeric protein each comprise one or more antigen-binding moieties. In such embodiments, each of the antigen-binding moieties can bind to the same or different antigens. In certain embodiments, the heterodimeric protein comprises: a first Fc polypeptide comprising a first antigen-binding moiety that binds to a first antigen; and a second Fc polypeptide comprising a second antigen-binding moiety that binds to a second antigen, wherein the first and second antigen are different (e.g., different molecules or different regions of the same molecule).

Any type of binding moiety that can be linked to an Fc polypeptide is suitable for use in the heterodimeric proteins disclosed herein. In certain embodiments, the binding moiety comprises an antibody variable domain. Exemplary binding moieties comprising an antibody variable domain include, without limitation, a VH, a VL, a VHH, a VH/VL pair, an scFv, a diabody, or a Fab. In certain embodiments, the binding moiety comprises a ligand. Exemplary ligands include, without limitation, hormones and growth factors, and analogues and mimetics thereof. In certain embodiments, the binding moiety comprises an extracellular domain of cell surface receptor. Exemplary cell surface receptors include, without limitation, a tumor necrosis factor superfamily receptor, a vascular endothelial growth factor receptor, or a transforming growth factor receptor. Such extracellular domains of cell surface receptors are well known in the art to be useful for sequestering the natural ligands of the receptors. Other binding moieties that are suitable for use in the heterodimeric proteins disclosed herein, include, without limitation, lipocalins (see e.g., Gebauer M. et al., 2012, Method Enzymol 503:157-188, which is incorporated by reference herein in its entirety), adnectins (see e.g., Lipovsek D., 2011, Protein Eng Des Sel 24:3-9, which is incorporated by reference herein in its entirety), avimers (see e.g., Silverman J, et al., 2005, Nat Biotechnol 23:1556-1561, which is incorporated by reference herein in its entirety), fynomers (see e.g., Schlatter D, et al., 2012, mAbs 4:497-508, which is incorporated by reference herein in its entirety), kunitz domains (see e.g., Hosse R. J. et al., 2006, Protein Sci 15:14-27, which is incorporated by reference herein in its entirety), knottins (see e.g., Kintzing J. R. et al., 2016, Curr Opin Chem Biol 34:143-150, which is incorporated by reference herein in its entirety), affibodies (see e.g., Feldwisch J. et al., 2010 J Mol Biol 398:232-247, which is incorporated by reference herein in its entirety), and DARPins (see e.g., Pluckthun A., 2015, Annu Rev Pharmacol Toxicol 55:489-511, which is incorporated by reference herein in its entirety).

In certain embodiments, the first and/or the second Fc polypeptides in a heterodimeric protein each comprises an antibody heavy chain. The antibody heavy chain can be a full-length antibody heavy chain or variant antibody heavy chain comprising one or more amino acid insertions, deletions, substitutions, or modifications relative to a naturally occurring full-length antibody heavy chain. In certain embodiments, the antibody heavy chain lacks a CH1 domain or comprises mutations in a CH1 domain or heavy chain variable domain that prevent association of the heavy chain with an antibody light chain. In certain embodiments, the antibody heavy chain lacks a portion of a hinge region.

Any species and isotype of antibody heavy chain can be used in the heterodimeric proteins disclosed herein. In certain embodiments, the first and/or the second Fc polypeptide comprises a human IgG₁, IgG₂, IgG₃, IgG₄, IgA₁, or IgA₂ antibody heavy chain or variant thereof. In certain embodiments, the first and/or the second Fc polypeptide comprises an antibody heavy chain that is a chimera of one or more human IgG₁, IgG₂, IgG₃, IgG₄, IgA₁, or IgA₂ antibody heavy chains.

In certain embodiments, the first and/or second Fc polypeptide comprises an antibody heavy chain and an antibody light chain (e.g., a human kappa or lambda light chain). The light chain can be linked (directly or indirectly) to any portion of the antibody heavy chain. In certain embodiments, the antibody heavy chain and the antibody light chain are linked to one another by disulphide bonds such that they form a half-antibody.

In certain embodiments, the heterodimeric protein comprises: a first Fc polypeptide comprising a first antibody heavy chain and a first antibody light chain that form a first half-antibody; and a second Fc polypeptide comprising a second antibody heavy chain and a second antibody light chain that form a second half-antibody. The first and the second half-antibodies can bind to the same antigen or different antigens (e.g., different molecules or different regions of the same molecule).

In certain embodiments, the CH3 domain of any of the first and/or second Fc polypeptides disclosed herein further comprises a C-terminal lysine. For example, in certain embodiments, any one of the CH3 domain-containing amino sequences disclosed in Table 1 can further comprise a lysine at the C-terminus of the CH3 domain.

In certain embodiments, the heterodimeric proteins disclosed herein exhibit improved biophysical properties (e.g., expression levels, solubility, thermal stability, manufacturability, and/or immunogenicity) relative to a corresponding prior art heterodimeric protein (e.g., multispecific antibody). In certain embodiments, the heterodimeric proteins disclosed herein exhibit improved thermal stability and manufacturability.

### 5.3 Pharmaceutical Compositions

Provided herein are compositions comprising a heterodimeric protein having the desired degree of purity in a physiologically acceptable carrier, excipient or stabilizer (see, e.g., Remington's Pharmaceutical Sciences (1990) Mack Publishing Co., Easton, Pa.). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In a specific embodiment, pharmaceutical compositions comprise a heterodimeric protein disclosed herein, and optionally one or more additional prophylactic or therapeutic agents, in a pharmaceutically acceptable carrier. In a specific embodiment, pharmaceutical compositions comprise an effective amount of a heterodimeric protein described herein, and optionally one or more additional prophylactic or therapeutic agents, in a pharmaceutically acceptable carrier. In certain embodiments, the heterodimeric protein is the only active ingredient included in the pharmaceutical composition. Pharmaceutical compositions described herein can be useful in treating a condition, such as cancer or an infectious disease. In one embodiment, the present invention relates to a pharmaceutical composition of the present invention comprising a heterodimeric protein of the present invention for use as a medicament. In another embodiment, the present invention relates to a pharmaceutical composition of the present invention for use in a method for the treatment of cancer or an infectious disease.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

A pharmaceutical composition may be formulated for any route of administration to a subject. Specific examples of routes of administration include intranasal, oral, pulmonary, transdermal, intradermal, and parenteral. Parenteral administration, characterized by either subcutaneous, intramuscular or intravenous injection, is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Preparations for parenteral administration of a heterodimeric protein include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Topical mixtures comprising a heterodimeric protein are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

A heterodimeric protein disclosed herein can be formulated as an aerosol for topical application, such as by inhalation (see, e.g., U.S. Pat. Nos. 4,044,126, 4,414,209 and 4,364,923, which describe aerosols for delivery of a steroid useful for treatment of inflammatory diseases, particularly asthma and are herein incorporated by reference in their entireties). These formulations for administration to the respiratory tract can be in the form of an aerosol or solution for a nebulizer, or as a microfine powder for insufflations, alone or in combination with an inert carrier such as lactose. In such a case, the particles of the formulation will, in one embodiment, have diameters of less than 50 microns, in one embodiment less than 10 microns.

A heterodimeric protein disclosed herein can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the heterodimeric protein alone or in combination with other pharmaceutically acceptable excipients can also be administered.

Transdermal patches, including iontophoretic and electrophoretic devices, are well known to those of skill in the art, and can be used to administer a heterodimeric protein. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957, all of which are herein incorporated by reference in their entireties.

In certain embodiments, a pharmaceutical composition comprising a heterodimeric protein described herein is a lyophilized powder, which can be reconstituted for administration as solutions, emulsions and other mixtures. It may also be reconstituted and formulated as solids or gels. The lyophilized powder is prepared by dissolving heterodimeric protein described herein, or a pharmaceutically acceptable derivative thereof, in a suitable solvent. In certain embodiments, the lyophilized powder is sterile. The solvent may contain an excipient which improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature. Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

Heterodimeric proteins and other compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874, all of which are herein incorporated by reference in their entireties. In a specific embodiment, a heterodimeric protein described herein is targeted to a tumor.

The compositions to be used for in vivo administration can be sterile. This is readily accomplished by filtration through, e.g., sterile filtration membranes.

### 5.4 Polynucleotides, Vectors and Methods of Producing Heterodimeric Proteins

In another aspect, provided herein are polynucleotides comprising a nucleotide sequence encoding Fc polypeptides described herein, and vectors, e.g., vectors comprising such polynucleotides for recombinant expression in host cells (e.g., E. coli and mammalian cells). Provided herein are polynucleotides comprising nucleotide sequences encoding the first and/or second Fc polypeptide of the heterodimeric proteins provided herein, as well as vectors comprising such polynucleotide sequences, e.g., expression vectors for their efficient expression in host cells, e.g., mammalian cells.

As used herein, an “isolated” polynucleotide or nucleic acid molecule is one which is separated from other nucleic acid molecules which are present in the natural source (e.g., in a mouse or a human) of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. For example, the language “substantially free” includes preparations of polynucleotide or nucleic acid molecule having less than about 15%, 10%, 5%, 2%, 1%, 0.5%, or 0.1% (in particular less than about 10%) of other material, e.g., cellular material, culture medium, other nucleic acid molecules, chemical precursors and/or other chemicals. In a specific embodiment, a nucleic acid molecule(s) encoding an Fc polypeptide described herein is isolated or purified.

In particular aspects, provided herein are polynucleotides comprising nucleotide sequences encoding Fc polypeptides. In certain aspects, provided herein are polynucleotides comprising a nucleotide sequence encoding the first and/or second Fc polypeptide of the heterodimeric proteins described herein.

Also provided herein are polynucleotides encoding Fc polypeptides that are optimized, e.g., by codon/RNA optimization, replacement with heterologous signal sequences, and elimination of mRNA instability elements. Methods to generate optimized nucleic acids encoding Fc polypeptides for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, all of which are herein incorporated by reference in their entireties. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In certain embodiments, it can be desirable to alter one or more codons to encode a conservative mutation, e.g., a similar amino acid with similar chemical structure and properties and/or function as the original amino acid. Such methods can increase expression of an Fc polyprotein by at least 1 fold, 2 fold, 3 fold, 4 fold, 5 fold, 10 fold, 20 fold, 30 fold, 40 fold, 50 fold, 60 fold, 70 fold, 80 fold, 90 fold, or 100 fold or more relative to the expression of an Fc polyprotein encoded by polynucleotides that have not been optimized.

In certain embodiments, an optimized polynucleotide sequence encoding an Fc polypeptide can hybridize to an antisense (e.g., complementary) polynucleotide of an unoptimized polynucleotide sequence encoding an Fc polypeptide described herein. In specific embodiments, an optimized nucleotide sequence encoding an Fc polypeptide described herein hybridizes under high stringency conditions to antisense polynucleotide of an unoptimized polynucleotide sequence encoding an Fc polypeptide described herein. In a specific embodiment, an optimized nucleotide sequence encoding an Fc polypeptide described herein hybridizes under high stringency, intermediate or lower stringency hybridization conditions to an antisense polynucleotide of an unoptimized nucleotide sequence encoding an Fc polypeptide described herein. Information regarding hybridization conditions has been described, see, e.g., U.S. Patent Application Publication No. US 2005/0048549 (e.g., paragraphs 72-73), which is herein incorporated by reference in its entirety.

The polynucleotides can be obtained, and the nucleotide sequence of the polynucleotides determined, by any method known in the art. Nucleotide sequences encoding Fc polypeptides described herein and modified versions of these Fc polypeptides can be determined using methods well known in the art, i.e., nucleotide codons known to encode particular amino acids are assembled in such a way to generate a nucleic acid that encodes the Fc polypeptide. Such a polynucleotide encoding the Fc polypeptide can be assembled from chemically synthesized oligonucleotides (e.g., as described in Kutmeier G et al., (1994), BioTechniques 17: 242-6, herein incorporated by reference in its entirety), which, briefly, involves the synthesis of overlapping oligonucleotides containing portions of the sequence encoding an Fc polypeptide, annealing and ligating of those oligonucleotides, and then amplification of the ligated oligonucleotides by PCR.

Alternatively, a polynucleotide encoding an Fc polypeptide described herein can be generated from nucleic acid from a suitable source using methods well known in the art (e.g., PCR and other molecular cloning methods). PCR amplification methods can be used to obtain nucleic acids comprising the sequence encoding the first and/or second Fc polypeptide of a heterodimeric protein. The amplified nucleic acids can be cloned into vectors for expression in host cells and for further cloning.

If a clone containing a nucleic acid encoding a particular Fc polypeptide is not available, but the sequence of the Fc polypeptide molecule is known, a nucleic acid encoding the Fc polypeptide can be chemically synthesized or obtained from a suitable source (e.g., a cDNA library generated from, or nucleic acid, preferably poly A+RNA, isolated from, any tissue or cells expressing the Fc polypeptide) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the Fc polypeptide. Amplified nucleic acids generated by PCR can then be cloned into replicable cloning vectors using any method well known in the art.

DNA encoding Fc polypeptides described herein can be readily isolated and sequenced using conventional procedures (e.g., by using oligonucleotide probes that are capable of binding specifically to genes encoding the heterodimeric protein. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce the Fc polypeptide, to obtain the synthesis of the Fc polypeptide in the recombinant host cells.

Also provided are polynucleotides that hybridize under high stringency, intermediate or lower stringency hybridization conditions to polynucleotides that encode an Fc polypeptide described herein. In specific embodiments, polynucleotides described herein hybridize under high stringency, intermediate or lower stringency hybridization conditions to polynucleotides encoding the first and/or second Fc polypeptide of a heterodimeric protein provided herein.

Hybridization conditions have been described in the art and are known to one of skill in the art. For example, hybridization under stringent conditions can involve hybridization to filter-bound DNA in 6× sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions are known to those of skill in the art and have been described, see, for example, Ausubel F M et al., eds., (1989) Current Protocols in Molecular Biology, Vol. I, Green Publishing Associates, Inc. and John Wiley & Sons, Inc., New York at pages 6.3.1-6.3.6 and 2.10.3, which is herein incorporated by reference in its entirety.

In certain aspects, provided herein are cells (e.g., host cells) expressing (e.g., recombinantly) Fc polypeptides described herein and related polynucleotides and expression vectors. Provided herein are vectors (e.g., expression vectors) comprising polynucleotides comprising nucleotide sequences encoding Fc polypeptides for recombinant expression in host cells, preferably in mammalian cells (e.g., CHO cells). Also provided herein are host cells comprising such vectors for recombinantly expressing Fc polypeptides described herein. In a particular aspect, provided herein are methods for producing Fc polypeptides described herein, comprising expressing such Fc polypeptides from a host cell.

Recombinant expression of Fc polypeptides described herein generally involves construction of an expression vector containing a polynucleotide that encodes the Fc polypeptide. Once a polynucleotide encoding an Fc polypeptide described herein has been obtained, the vector for the production of the Fc polypeptide molecule can be produced by recombinant DNA technology using techniques well known in the art. Thus, methods for preparing a protein by expressing a polynucleotide containing an Fc polypeptide encoding nucleotide sequence are described herein. Methods which are well known to those skilled in the art can be used to construct expression vectors containing Fc polypeptide coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding an Fc polypeptide described herein operably linked to a promoter.

An expression vector can be transferred to a cell (e.g., host cell) by conventional techniques and the resulting cells can then be cultured by conventional techniques to produce an Fc polypeptide described herein. Thus, provided herein are host cells containing a polynucleotide encoding an Fc polypeptide described herein operably linked to a promoter for expression of such sequences in the host cell. In certain embodiments, vectors encoding both the first and second Fc polypeptide of a heterodimeric protein, individually, can be co-expressed in the host cell for expression of the entire heterodimeric protein, as detailed below. In certain embodiments, a host cell contains a vector comprising a polynucleotide encoding both the first and second Fc polypeptide of a heterodimeric protein described herein. In specific embodiments, a host cell contains two different vectors, a first vector comprising a polynucleotide encoding a first Fc polypeptide of a heterodimeric protein described herein and a second vector comprising a polynucleotide encoding a second Fc polypeptide of a heterodimeric protein. In certain embodiments, a host cell comprises the following four vectors: a first vector comprising a polynucleotide encoding a first antibody heavy chain of a first half-antibody of a heterodimeric protein described herein; a second vector comprising a polynucleotide encoding a second antibody heavy chain of a second half-antibody of a heterodimeric protein described herein; a third vector comprising a polynucleotide encoding a first antibody light chain of a first half-antibody of a heterodimeric protein described herein; and a fourth vector comprising a polynucleotide encoding a second antibody light chain of a second half-antibody of a heterodimeric protein described herein. In certain embodiments, vectors encoding the first antibody heavy chain of a first half-antibody, the second antibody heavy chain of a second half-antibody, the first antibody light chain of a first half-antibody, and the second antibody light chain of a second half-antibody, individually, can be co-expressed in the host cell to produce the heterodimeric protein.

In other embodiments, a first host cell comprises a first vector comprising a polynucleotide encoding a first Fc polypeptide of a heterodimeric protein described herein, and a second host cell comprises a second vector comprising a polynucleotide encoding a second Fc polypeptide of a heterodimeric protein described herein. In specific embodiments, a first Fc polypeptide expressed by a first cell associates with a second Fc polypeptide of a second cell to form a heterodimeric protein described herein. In certain embodiments, a first host cell comprises a first vector comprising a polynucleotide encoding a first antibody heavy chain of a first half-antibody of a heterodimeric protein described herein and a second vector comprising a polynucleotide encoding a first antibody light chain of a first half-antibody of a heterodimeric protein described herein, and a second host cell comprises a third vector comprising a polynucleotide encoding a second antibody heavy chain of a second half-antibody of a heterodimeric protein described herein and a fourth vector comprising a polynucleotide encoding a second antibody light chain of a second half-antibody of a heterodimeric protein described herein. In certain embodiments, a first half-antibody expressed by a first cell associates with a second half-antibody of a second cell to form a heterodimeric protein described herein. In certain embodiments, provided herein is a population of host cells comprising such first host cell and such second host cell.

A variety of host-expression vector systems can be utilized to express Fc polypeptides described herein (see, e.g., U.S. Pat. No. 5,807,715, which is herein incorporated by reference in its entirety). Such host-expression systems represent vehicles by which the coding sequences of interest can be produced and subsequently purified, but also represent cells which can, when transformed or transfected with the appropriate nucleotide coding sequences, express heterodimeric protein molecules described herein in situ. These include but are not limited to microorganisms such as bacteria (e.g., E. coli and B. subtilis) transformed with, e.g., recombinant bacteriophage DNA, plasmid DNA or cosmid DNA expression vectors containing heterodimeric protein coding sequences; yeast (e.g., Saccharomyces Pichia) transformed with, e.g., recombinant yeast expression vectors containing heterodimeric protein coding sequences; insect cell systems infected with, e.g., recombinant virus expression vectors (e.g., baculovirus) containing heterodimeric protein coding sequences; plant cell systems (e.g., green algae such as Chlamydomonas reinhardtii) infected with, e.g., recombinant virus expression vectors (e.g., cauliflower mosaic virus, CaMV; tobacco mosaic virus, TMV) or transformed with, e.g., recombinant plasmid expression vectors (e.g., Ti plasmid) containing heterodimeric protein coding sequences; or mammalian cell systems (e.g., COS (e.g., COS1 or COS), CHO, BHK, MDCK, HEK 293, NS0, PER.C6, VERO, CRL7O3O, HsS78Bst, HeLa, and NIH 3T3, HEK-293T, HepG₂, SP210, R1.1, B-W, L-M, BSC1, BSC40, YB/20 and BMT10 cells) harboring, e.g., recombinant expression constructs containing promoters derived from the genome of mammalian cells (e.g., metallothionein promoter) or from mammalian viruses (e.g., the adenovirus late promoter; the vaccinia virus 7.5K promoter). In a specific embodiment, cells for expressing heterodimeric proteins described herein are Chinese hamster ovary (CHO) cells, for example CHO cells from the CHO GS System™ (Lonza). In a particular embodiment, cells for expressing heterodimeric proteins described herein are human cells, e.g., human cell lines. In a specific embodiment, a mammalian expression vector is pOptiVEC™ or pcDNA3.3. In a particular embodiment, bacterial cells such as Escherichia coli, or eukaryotic cells (e.g., mammalian cells) are used for the expression of heterodimeric proteins. For example, mammalian cells such as CHO cells, in conjunction with a vector such as the major intermediate early gene promoter element from human cytomegalovirus is an effective expression system for proteins, such as antibodies (Foecking M K & Hofstetter H (1986) Gene 45: 101-5; and Cockett M I et al., (1990) Biotechnology 8(7): 662-7, each of which is herein incorporated by reference in its entirety). In certain embodiments, heterodimeric proteins described herein are produced by CHO cells or NSO cells. In a specific embodiment, the expression of nucleotide sequences encoding heterodimeric proteins described herein is regulated by a constitutive promoter, inducible promoter or tissue specific promoter.

In bacterial systems, a number of expression vectors can be advantageously selected depending upon the use intended for the heterodimeric protein being expressed. For example, when a large quantity of such protein is to be produced for the generation of pharmaceutical compositions of heterodimeric proteins, vectors which direct the expression of high levels of fusion protein products that are readily purified can be desirable. Such vectors include, but are not limited to, the E. coli expression vector pUR278 (Ruether U & Mueller-Hill B (1983) EMBO J 2: 1791-1794), in which the protein coding sequence can be ligated individually into the vector in frame with the lac Z coding region so that a fusion protein is produced; pIN vectors (Inouye S & Inouye M (1985) Nuc Acids Res 13: 3101-3109; Van Heeke G & Schuster S M (1989) J Biol Chem 24: 5503-5509); and the like, all of which are herein incorporated by reference in their entireties. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV), for example, can be used as a vector to express foreign genes. The virus grows in Spodoptera frugiperda cells. The protein coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the protein coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the heterodimeric protein in infected hosts (e.g., see Logan J & Shenk T (1984) PNAS 81(12): 3655-9, which is herein incorporated by reference in its entirety). Specific initiation signals can also be required for efficient translation of inserted protein coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al., (1987) Methods Enzymol. 153: 516-544, which is herein incorporated by reference in its entirety).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT2O and T47D, NSO (a murine myeloma cell line that does not endogenously produce any immunoglobulin chains), CRL7O3O, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells. In certain embodiments, heterodimeric proteins described herein are produced in mammalian cells, such as CHO cells.

In a specific embodiment, the heterodimeric proteins described herein have reduced fucose content or no fucose content. Such proteins can be produced using techniques known to one skilled in the art. For example, the Fc polypeptides described herein can be expressed in cells deficient or lacking the ability of to fucosylate. In a specific example, cell lines with a knockout of both alleles of a1,6-fucosyltransferase can be used to produce Fc polypeptides with reduced fucose content. The Potelligent® system (Lonza) is an example of such a system that can be used to produce Fc polypeptides with reduced fucose content.

For long-term, high-yield production of recombinant proteins, stable expression cells can be generated. For example, cell lines which stably express Fc polypeptides described herein can be engineered. In specific embodiments, a cell provided herein stably expresses a first Fc polypeptide and a second Fc polypeptide which associate to form a heterodimeric protein described herein. In certain embodiments, a first cell provided herein stably expresses a first Fc polypeptide and second cell provided herein stably expresses a second Fc polypeptide. In certain embodiments, a first Fc polypeptide expressed by a first cell associates with a second Fc polypeptide of a second cell to form a heterodimeric protein described herein.

In certain aspects, rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA/polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express Fc polypeptides described herein. Such engineered cell lines can be particularly useful in screening and evaluation of compositions that interact directly or indirectly with the Fc polypeptides.

A number of selection systems can be used, including but not limited to the herpes simplex virus thymidine kinase (Wigler M et al., (1977) Cell 11(1): 223-32), hypoxanthineguanine phosphoribosyltransferase (Szybalska E H & Szybalski W (1962) PNAS 48(12): 2026-2034) and adenine phosphoribosyltransferase (Lowy I et al., (1980) Cell 22(3): 817-23) genes in tk-, hgprt- or aprt-cells, respectively, all of which are herein incorporated by reference in their entireties. Also, antimetabolite resistance can be used as the basis of selection for the following genes: dhfr, which confers resistance to methotrexate (Wigler M et al., (1980) PNAS 77(6): 3567-70; O'Hare K et al., (1981) PNAS 78: 1527-31); gpt, which confers resistance to mycophenolic acid (Mulligan R C & Berg P (1981) PNAS 78(4): 2072-6); neo, which confers resistance to the aminoglycoside G-418 (Wu G Y & Wu C H (1991) Biotherapy 3: 87-95; Tolstoshev P (1993) Ann Rev Pharmacol Toxicol 32: 573-596; Mulligan RC (1993) Science 260: 926-932; and Morgan R A & Anderson W F (1993) Ann Rev Biochem 62: 191-217; Nabel G J & Felgner P L (1993) Trends Biotechnol 11(5): 211-5); and hygro, which confers resistance to hygromycin (Santerre R F et al., (1984) Gene 30(1-3): 147-56), all of which are herein incorporated by reference in their entireties. Methods commonly known in the art of recombinant DNA technology can be routinely applied to select the desired recombinant clone and such methods are described, for example, in Ausubel F M et al., (eds.), Current Protocols in Molecular Biology, John Wiley & Sons, NY (1993); Kriegler M, Gene Transfer and Expression, A Laboratory Manual, Stockton Press, NY (1990); and in Chapters 12 and 13, Dracopoli N C et al., (eds.), Current Protocols in Human Genetics, John Wiley & Sons, NY (1994); Colbère-Garapin F et al., (1981) J Mol Biol 150: 1-14, all of which are herein incorporated by reference in their entireties.

The expression levels of protein can be increased by vector amplification (for a review, see Bebbington C R & Hentschel C C G, The use of vectors based on gene amplification for the expression of cloned genes in mammalian cells in DNA cloning, Vol. 3 (Academic Press, New York, 1987), which is herein incorporated by reference in its entirety). When a marker in the vector system expressing an Fc polypeptide is amplifiable, increase in the level of inhibitor present in culture of host cell will increase the number of copies of the marker gene. Since the amplified region is associated with the Fc polypeptide gene, production of the Fc polypeptide will also increase (Crouse G F et al., (1983) Mol Cell Biol 3: 257-66, which is herein incorporated by reference in its entirety).

The host cell can be co-transfected with two or more expression vectors described herein, the first vector encoding a first Fc polypeptide and the second vector encoding a second Fc polypeptide. The two vectors can contain identical selectable markers which enable equal expression of first and second Fc polypeptides. The host cells can be co-transfected with different amounts of the two or more expression vectors. For example, host cells can be transfected with any one of the following ratios of a first expression vector and a second expression vector: about 1:1, 1:2, 1:3, 1:4, 1:5, 1:6, 1:7, 1:8, 1:9, 1:10, 1:12, 1:15, 1:20, 1:25, 1:30, 1:35, 1:40, 1:45, or 1:50.

Alternatively, a single vector can be used which encodes, and is capable of expressing, both the first and second Fc polypeptides of a heterodimeric protein. The coding sequences for the first and second Fc polypeptides can comprise cDNA or genomic DNA. The expression vector can be monocistronic or multicistronic. A multicistronic nucleic acid construct can encode 2, 3, 4, 5, 6, 7, 8, 9, 10 or more genes/nucleotide sequences, or in the range of 2-5, 5-10, or 10-20 genes/nucleotide sequences. For example, a bicistronic nucleic acid construct can comprise, in the following order, a promoter, a first gene (e.g., first Fc polypeptide of a heterodimeric protein described herein), and a second gene and (e.g., second Fc polypeptide of a heterodimeric protein described herein). In such an expression vector, the transcription of both genes can be driven by the promoter, whereas the translation of the mRNA from the first gene can be by a cap-dependent scanning mechanism and the translation of the mRNA from the second gene can be by a cap-independent mechanism, e.g., by an IRES.

Once a heterodimeric protein or Fc polypeptide described herein has been produced by recombinant expression, it can be purified by any method known in the art for protein purification, for example, by chromatography (e.g., ion exchange, affinity, and sizing column chromatography), centrifugation, differential solubility, or by any other standard technique for the purification of proteins. Further, the heterodimeric proteins and Fc polypeptides described herein can be fused to heterologous polypeptide sequences described herein or otherwise known in the art to facilitate purification.

In specific embodiments, a heterodimeric protein or Fc polypeptide described herein is isolated or purified. Generally, an isolated protein is one that is substantially free of other proteins. For example, in a particular embodiment, a preparation of a heterodimeric protein or Fc polypeptides described herein is substantially free of cellular material and/or chemical precursors. The language “substantially free of cellular material” includes preparations of a protein in which the protein is separated from cellular components of the cells from which it is isolated or recombinantly produced. Thus, a protein that is substantially free of cellular material includes preparations of protein having less than about 30%, 20%, 10%, 5%, 2%, 1%, 0.5%, or 0.1% (by dry weight) of heterologous protein (also referred to herein as a “contaminating protein”) and/or variants of a protein, for example, different post-translational modified forms of a protein or other different versions of a protein (e.g., protein fragments). When the protein is recombinantly produced, it is also generally substantially free of culture medium, i.e., culture medium represents less than about 20%, 10%, 2%, 1%, 0.5%, or 0.1% of the volume of the protein preparation. When the protein is produced by chemical synthesis, it is generally substantially free of chemical precursors or other chemicals, i.e., it is separated from chemical precursors or other chemicals which are involved in the synthesis of the protein. Accordingly, such preparations of the protein have less than about 30%, 20%, 10%, or 5% (by dry weight) of chemical precursors or compounds other than the protein of interest. In a specific embodiment, heterodimeric proteins and Fc polypeptides described herein are isolated or purified.

Heterodimeric proteins and Fc polypeptides described herein can be produced by any method known in the art for the synthesis of proteins, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press, all of which are herein incorporated by reference in their entireties.

### 6. EXAMPLES

The examples are offered by way of illustration and not by way of limitation.

### 6.1 Example 1: Synthesis and Heterodimerization of Fc Polypeptides

This example describes the synthesis and heterodimerization of Fc polypeptides specific for Target 1 or Target 2 (two different antigens present on the surface of human T cells).

### 6.1.1 Fc Polypeptide Synthesis

Twelve Fc polypeptides (each comprising a half-antibody specific for either Target 1 or Target 2) were expressed in CHO cells and purified using Protein A affinity chromatography (GE Healthcare). The binding specificity of the antigen binding portion, and the amino acid sequence of the heavy chain constant region, for each of the twelve Fc polypeptides is described in Table 3.

TABLE 3
Fc polypeptides

| | | SEQ ID NO of |
|---|---|---|
| Fc | | heavy chain |
| polypeptide | | constant region |
| # | Antigen specificity and Fc mutation(s) | sequence* |
| 1 | Target 1 T366W | 22 |
| 2 | Target 1 T366S/L368A/Y407V | 30 |
| 3 | Target 1 S239D/A330L/I332E/T366W | 24 |
| 4 | Target 1 S239D/A330L/I332E/ | 32 |
| | T366S/L368A/Y407V | |
| 5 | Target 1 S239D/T366S/L368A/Y407V | 36 |
| 6 | Target 1 wild-type IgG1 | 14 |
| 7 | Target 2 T366W | 22 |
| 8 | Target 2 T366S/L368A/Y407V | 30 |
| 9 | Target 2 S239D/A330L/I332E/T366W | 24 |
| 10 | Target 2 S239D/A330L/I332E/ | 32 |
| | T366S/L368A/Y407V | |
| 11 | Target 2 S239D/T366S/L368A/Y407V | 36 |
| 12 | Target 2 wild-type IgG1 | 14 |

*The nucleic acid coding sequence for each of these heavy chain constant regions includes a lysine at the C-terminus.

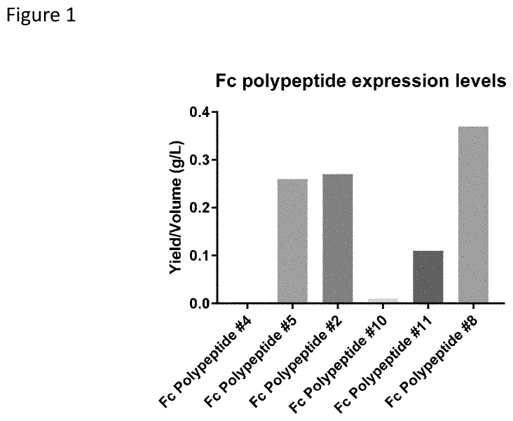

Protein expression levels for Fc polypeptide #s 4, 5, 2, 10, 11, and 8 were measured and compared. As shown in FIG. 1, Fc polypeptides 4 and 10 (which contained both S239D/A330L/I332E mutations and T366S/L368A/Y407V mutations) were expressed at negligible levels relative to the other Fc polypeptides tested.

### 6.1.2 Heterodimerization of Fc Polypeptides

The Fc polypeptides set forth in Table 3 were heterodimerized to form the heterodimeric proteins described in Table 4. Briefly, the first and second Fc polypeptides indicated in Table 4 were mixed in equimolar amounts in the presence of 50 mM of reductant (2-MEA) for at least 2 hours to reduce heavy chain interchain disulphide bonds. The mixtures of Fc polypeptides were then buffer-exchanged into reoxidation buffer (10 mM sodium citrate pH 6.0, 115 mM NaCl) using desalting PD-10 columns to remove the reductant and incubated at room temperature for 24 hours to facilitate dimerization of the first and second Fc polypeptides. The resultant Fc polypeptide mixtures were then buffer-exchanged into storage buffer (10 mM histidine pH 6 and 115 mM NaCl). Purity and quality were determined by SDS-PAGE and dimer formation and percent heterodimerization was assessed by absolute size-exclusion chromatography (ASEC).

TABLE 4
Heterodimeric proteins

| | First | Second |
|---|---|---|
| Name | Fc polypeptide | Fc polypeptide |
| BA111 | Fc polypeptide #1 | Fc polypeptide #2 |
| BA112 | Fc polypeptide #3 | Fc Polypeptide #2 |
| BA113 | Fc polypeptide #3 | Fc polypeptide #5 |
| BA114 | Fc polypeptide #3 | Fc polypeptide #4 |
| BA115 | Fc polypeptide #7 | Fc polypeptide #8 |
| BA116 | Fc polypeptide #9 | Fc polypeptide #8 |
| BA117 | Fc polypeptide #9 | Fc polypeptide #11 |
| BA118 | Fc polypeptide #9 | Fc polypeptide #10 |

### 6.1.3 Thermal Stability

The thermal stability of heterodimeric protein BA111, BA112, BA113, BA114, BA115, BA116, BA117, and BA118 was assessed. 1 μl of Sypro Orange Fluorescent Dye (final Sypro concentration at 5×) was added to 4 μg of protein diluted in PBS. The Sypro Orange fluorescence was analyzed on a Thermal Cycler (Biorad CFX96 Real-Time System C1000 PCR Detection System) using a thermal ramp starting at 10° C. to 95° C. and the protein thermal shifts were analyzed on the CFX Maestro software. Bar charts were created in prism.

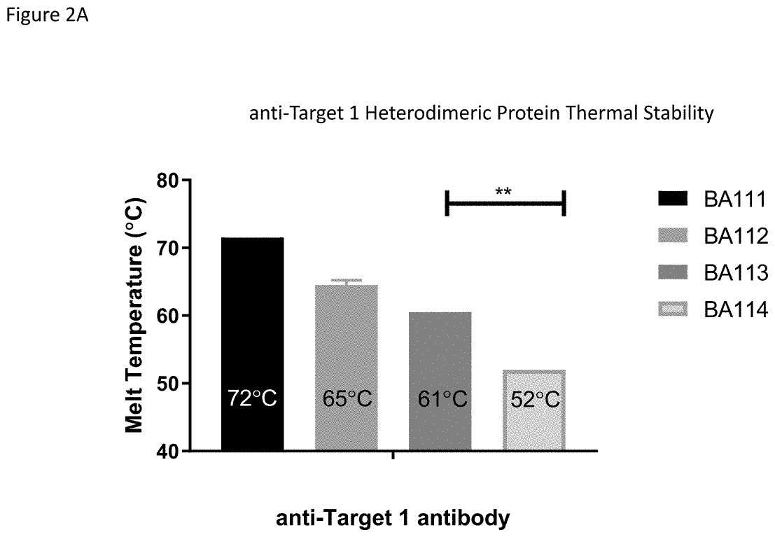

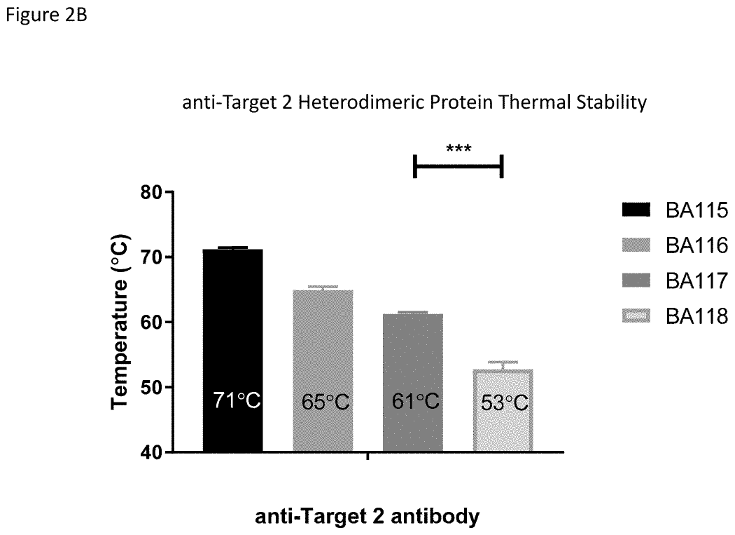

FIG. 2A shows the melting temperature for anti-Target 1 heterodimeric proteins BA111, BA112, BA113, and BA114. The melting temperature of BA114 is lower than BA111, BA112, or BA113.

FIG. 2B shows the melting temperature for anti-Target 2 heterodimeric proteins BA115, BA116, BA117, and BA118. The melting temperature of BA118 is lower than BA115, BA116, or BA117.

### 6.2 Example 2: Target Binding

This example demonstrates the ability of the heterodimeric proteins set forth in Table 4 to bind to their intended targets.

### 6.2.1 Target 1

The ability of heterodimeric proteins BA111, BA112, BA113, BA114, homodimeric protein BA119 (WT IgG₁), and an IgG₁isotype control antibody to bind to Target 1 was assessed.

Briefly, a frozen aliquot of Jurkat cells engineered to express human Target 1 was thawed at 37° C. and cultured overnight in RPMI media supplemented with 10% fetal bovine serum (FBS) at 37° C. and 5% CO2. Cells were counted and their viability assessed using a Muse apparatus (Luminex Corp.) by mixing a 20 μl aliquot of cells with 380 μl of viability dye. Cells were then pelleted by centrifugation at 1500 rpm for five minutes and resuspended to a final concentration of 1×10⁶ cells/mL in 1× phosphate buffer saline (PBS) supplemented with 2% FBS (FACS buffer). Cells were seeded in a 96-well U-bottom tissue culture plate at a density of 1×10⁵ cells per well and washed twice with FACS buffer.

Heterodimeric proteins were serially diluted 1:10 in FACS buffer for a total of 8 working dilutions ranging from 50 μg/ml to 0.000005 μg/ml. Cells were resuspended in 50 μl of the heterodimeric protein solution, and incubated for 30 minutes at 4° C.

To detect heterodimeric protein binding, cells were washed twice with cold FACS Buffer and resuspended in FACS Buffer containing fluorescein isothiocyanate (FITC)-labeled goat anti-human IgG (H+L) secondary antibody at a 1:200 final dilution and live/dead staining solution at a 1:1000 final dilution. Cells were then incubated for 30 minutes at 4° C., washed twice with cold FACS Buffer, and analyzed by flow cytometry (BD LSR Fortessa Flow Cytometer). The data was analyzed using FlowJo (Version 10, FLOWJO) software by sequentially gating the FSC-A vs. SSC-A, SSC-H vs SSC-A, and SSC-A vs. live-dead. Mean fluorescence intensity (MFI) values for FITC staining were calculated, and the data was plotted using GraphPad Prism software (Version 7, GraphPad Software Inc.).

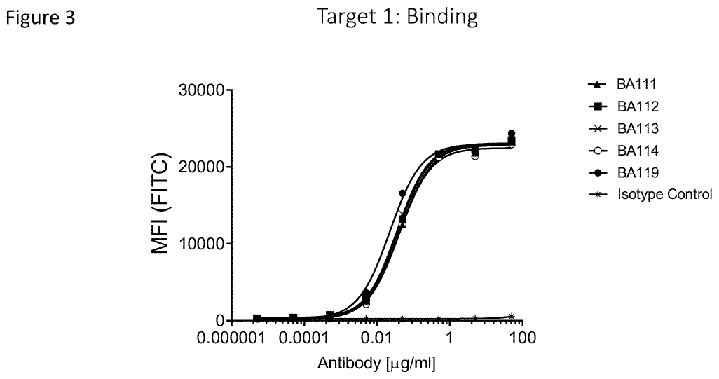

As shown in FIG. 3, BA111, BA112, BA113, BA114, and BA119 showed potent and comparable binding to Target 1-expressing cells.

### 6.2.2 Target 2

The ability of heterodimeric proteins BA115, BA116, BA117, BA118, homodimeric protein BA120 (WT IgG₁), and an IgG₁isotype control antibody to bind Target 2 was assessed.

Briefly, a frozen aliquot of CHO cells engineered to express Target 2 was thawed at 37° C. and cultured overnight in F-12 media supplemented with 10% fetal bovine serum (FBS) at 37° C. and 5% CO2. Cells were counted and their viability assessed using a Vi-XCell apparatus. Cells were then pelleted by centrifugation at 1500 rpm for five minutes and resuspended to a final concentration of 1×10⁶ cells/mL in 1× phosphate buffer saline (PBS) supplemented with 2% FBS (FACS buffer). Cells were seeded in a 96-well U-bottom tissue culture plate at a density of 1×10⁵ cells per well and washed twice with FACS buffer.

Heterodimeric proteins were serially diluted 1:3 in FACS buffer for a total of 8 working dilutions ranging from 30 μg/ml to 0.013717 μg/ml. Cells were resuspended in 50 μl of the heterodimeric protein solution, and incubated for 30 minutes at 4° C.

To detect heterodimeric protein binding, cells were washed twice with cold FACS Buffer and resuspended in FACS Buffer containing fluorescein isothiocyanate (FITC)-labeled goat anti-human IgG (H+L) secondary antibody at a 1:200 final dilution and live/dead staining solution at a 1:1000 final dilution. Cells were then incubated for 30 minutes at 4° C., washed twice with cold FACS Buffer, and analyzed by flow cytometry (BD LSR Fortessa Flow Cytometer). The data was analyzed using the FlowJo software by sequentially gating the FSC-A vs. SSC-A, SSC-H vs SSC-A, and SSC-A vs. live-dead. Mean fluorescence intensity (MFI) values for FITC staining were calculated, and the data was plotted using GraphPad Prism software.

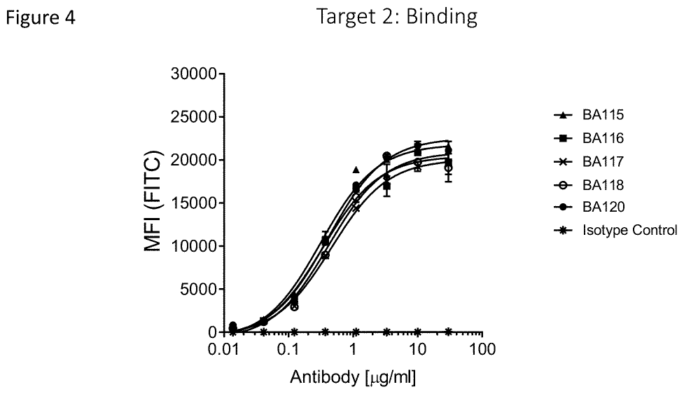

As shown in FIG. 4, BA115, BA116, BA117, BA118, and BA120 showed potent and comparable binding to Target 2-expressing cells.

### 6.3 Example 3: Target Blockade

This example demonstrates the ability of the heterodimeric proteins set forth in Table 4 to block signaling in cells expressing their intended target.

### 6.3.1 Target 1

The ability of heterodimeric proteins BA111, BA112, BA113, BA114, homodimeric protein BA119 (WT IgG₁), and an IgG₁isotype control antibody to block signaling by binding Target 1 was assessed using a Jurkat reporter assay.

Briefly, a human T cell line (Jurkat) engineered to express human Target 1 with a luciferase reporter driven by a native promoter that can respond to both TCR activation and inhibitory co-receptor signaling that suppresses activation (Promega) was plated in a 96 well flat-bottom white plate according to the manufacturer's protocol and treated with a dose-titration of BA111, BA112, BA113, BA114, BA119, and an IgG₁isotype control antibody, serially diluted 1-to-2.5 in culture media from 50 μg/ml to 0.015 μg/ml. To assess the functional activity of the indicated heterodimeric proteins, CHO-K1 cells engineered to express the natural ligand of Target 1 and an engineered cell surface protein designed to activate the T cell receptor (TCR) complex in an antigen-independent manner were co-cultured with the heterodimeric protein-treated Jurkat reporter cell line for 6 hours at 37° C. and 5% CO2. To assess the ability of the heterodimeric proteins to block the interaction of Target 1 with its natural ligand and enhance T cell activation gene activity, luciferase expression was quantified using Bio-Glo™ reagent and an Envision plate-reading luminometer.

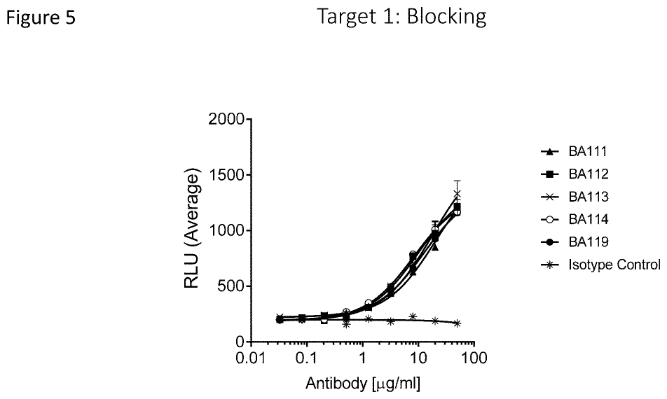

As shown in FIG. 5, co-culture of the cell lines resulted in engagement of the inhibitory co-receptor of Target 1 (expressed on Jurkat cells) with its natural ligands CD80 and CD86 (expressed on Raji cells), which inhibited T cell activation, as shown by a lack of luciferase expression. This inhibition was relieved upon addition of increasing concentrations of BA111, BA112, BA113, BA114, or the homodimeric protein BA119. BA111, BA112, BA113, BA114 and BA119 were functionally comparable at enhancing T cell activation, as determined by IL-2 reporter gene activity.

### 6.3.2 Target 2

The ability of heterodimeric proteins BA115, BA116, BA117, BA118, homodimeric protein BA120 (WT IgG₁), and an IgG₁isotype control antibody to block signaling by binding Target 2 was assessed using a Jurkat reporter assay.

Briefly, a human T cell line (Jurkat) that endogenously expresses the inhibitory co-receptor of Target 2 was engineered to constitutively express cell surface Target 2 and a luciferase reporter gene driven by an IL-2 promoter (Promega). Cells were plated in a 96 well flat-bottom white plate according to the manufacturer's protocol and treated with a dose-titration of the indicated anti-Target 2 heterodimeric proteins or an isotype control IgG₁antibody. Heterodimeric proteins were serially diluted 1:2.5 in culture media with concentrations ranging from 50 μg/ml to 0.015 μg/ml. An antigen presenting cell line (Raji) that endogenously expresses the natural ligands of Target 2, and was engineered to express a proprietary T cell activator, was prepared according to the manufacturer's instructions and co-cultured with the heterodimeric protein-treated Jurkat cells for 6 hours at 37° C. and 5% CO2. To assess the ability of the heterodimeric proteins to block Target 2 interactions and enhance IL-2 reporter gene activity, luciferase expression was quantified using Bio-Glo™ reagent and an Envision plate-reading luminometer.

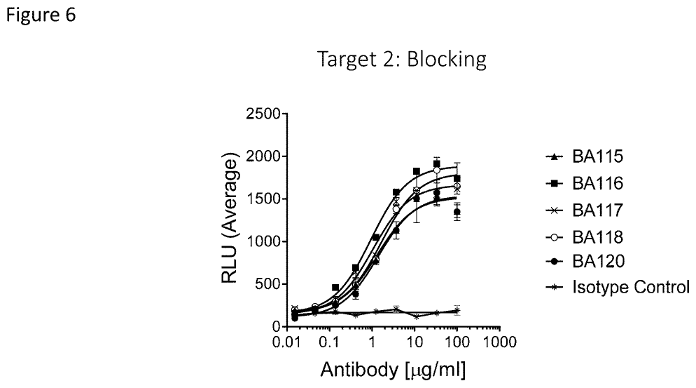

As shown in FIG. 6, co-culture of the two cell lines resulted in engagement of the inhibitory co-receptor of Target 2 (expressed on Jurkat cells) with its natural ligands (expressed on Raji cells), which inhibited T cell activation, as shown by a lack of luciferase expression. This inhibition was relieved upon addition of increasing concentrations of BA115, BA116, BA117, BA118, or the homodimeric protein BA120. BA115, BA116, BA117, BA118, and BA120 were functionally comparable at enhancing T cell activation, as determined by IL-2 reporter gene activity.

### 6.4 Example 4: SEA Stimulation Assay

This example demonstrates the ability of heterodimeric proteins to elicit IL-2 secretion by SEA-stimulated PBMCs.

### 6.4.1 Target 1

The ability of heterodimeric proteins specific for Target 1 to elicit IL-2 secretion by SEA-stimulated PBMCs was investigated.

Briefly, a 5× concentrated intermediate stock of heterodimeric protein sufficient for three replicates per donor was prepared in 1.2 mL bullet tubes. First, 420 μL of 500 μg/mL of each heterodimeric protein was prepared in R10 media. Heterodimeric proteins were then serially diluted 1-to-10 for a total of 8 dilutions and 20 μl of heterodimeric protein mixture was then added to corresponding wells of a round-bottom 96-well plate. Frozen aliquots of human PBMCs were retrieved from liquid nitrogen and immediately thawed in 37° C. water. Cells were transferred to 9 mL of pre-warmed R10 media and immediately centrifuged at 2000 rpm for two minutes. Cells were then counted, and viability was assessed. Cells were centrifuged at 2000 rpm for two minutes and resuspended.

An intermediate stock concentration of SEA was made by diluting 10 μL of 10 μg/mL of SEA in 90 μL of R10 to make an intermediate concentration of 1 μg/mL. To stimulate the cells, 35 μL of the 1 μg/mL intermediate stock of SEA was added to 28 mL of cells. 80 μL of cells and SEA mixture was added into corresponding wells and incubated in a humidified chamber at 37° C. and 5% CO₂ for four days. A total of 0.1×10⁶ cells/well and a final concentration of 1 ng/mL of SEA was used.

After four days of incubation, plates were removed from the incubator, gently agitated by hand, and then centrifuged for two minutes at 2000 rpm. 5 μL of the supernatant was transferred to a 384-well AlphaLISA plate (Perkin Elmer) for cytokine analysis. AlphaLISA kits were used for the measurements of IL-2 in accordance with manufacturer instructions. Briefly, assay buffer was prepared by adding 2.5 mL of 10× AlphaLISA Immunoassay Buffer to 22.5 mL water. Human IL-2 analyte was used to prepare a standard dilution in accordance with manufacturer instructions. A mixture of 1.6× AlphaLISA anti-IL-2 acceptor beads and biotinylated antibody anti-IL-2 mix was prepared in assay buffer. 8 μL was added to each well and incubated in darkness at room temperature, rotating at 500 rpm for 90 minutes. A 2.3× streptavidin donor bead intermediate stock was prepared in assay buffer. 10 μL was added to each well and incubated in darkness at room temperature, rotating at 500 rpm for 20 minutes. AlphaLISA plates were briefly centrifuged at 2000 rpm. Relative light units (RLU) were measured using the AlphaScreen protocol on an EnVision Plate Reader.

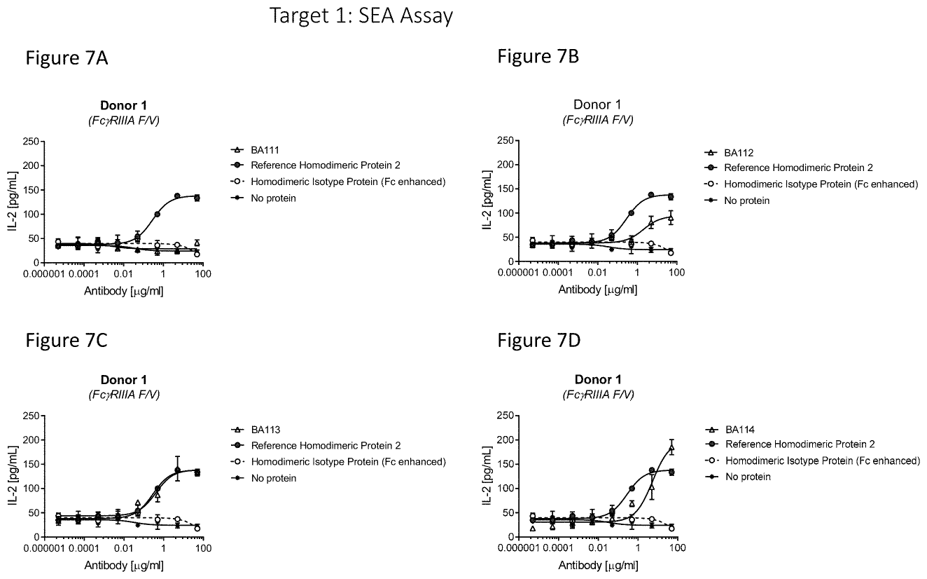

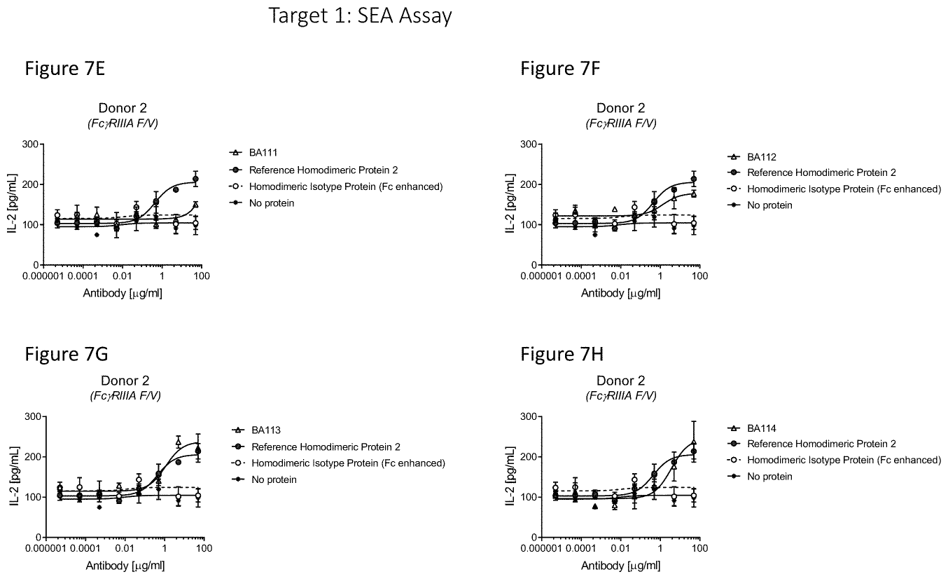

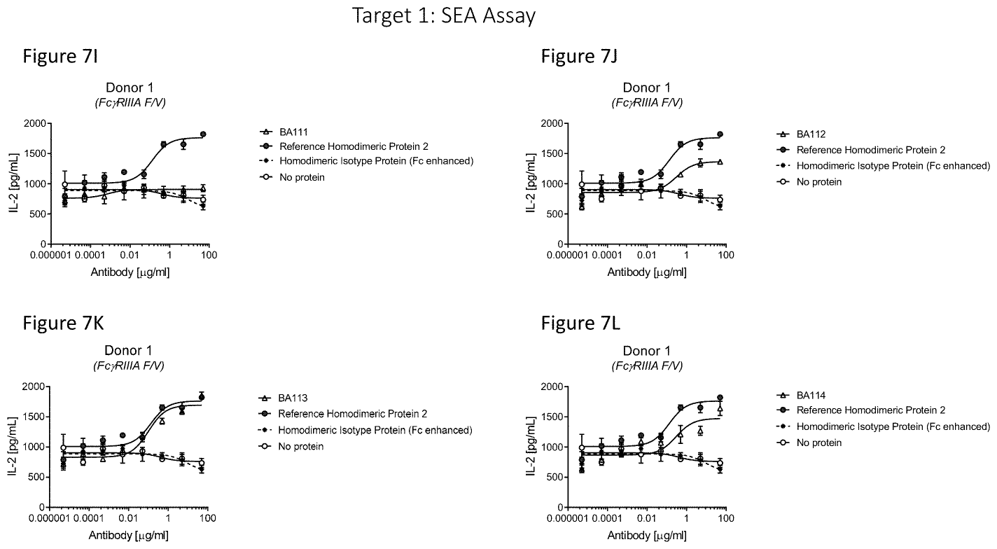

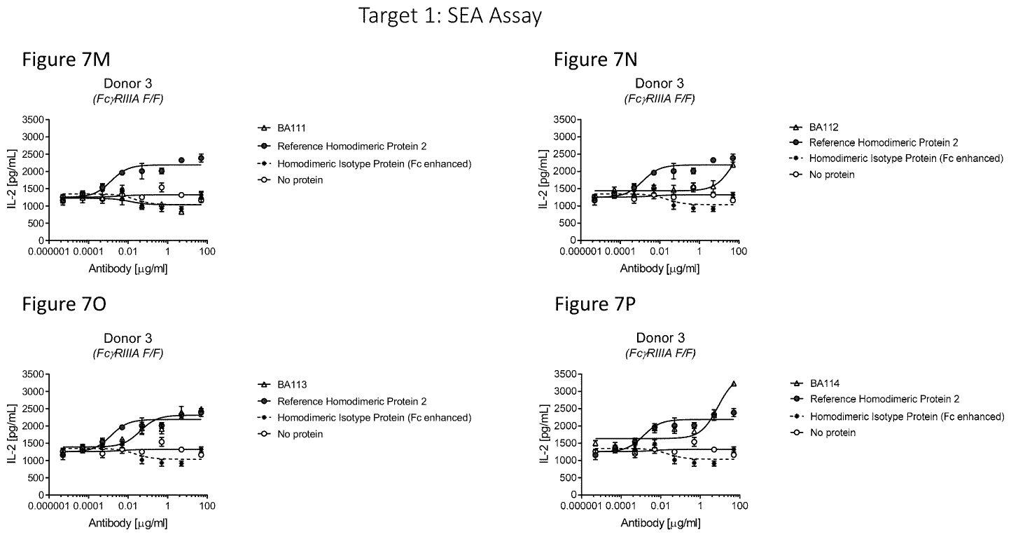

As shown in FIGS. 7A-7P, BA113 potently enhanced IL-2 secretion in a dose-dependent manner that was comparable to Reference Homodimeric Protein 2, also specific for Target 1. BA113 demonstrated superior functional activity compared BA112 and BA111. BA111 did not induce significant levels of IL-2 secretion over cells treated with a homodimeric isotype control protein, which comprises S239D/A330L/I332E mutations (“Fc enhanced”), and was used as an isotype control. BA114 showed an apparent reduced potency in enhancing IL-2 secretion compared to BA113. The results presented in FIGS. 7A-7P were obtained using PBMCs from 3 different donors, Donor 1 (FIGS. 7A-7D and FIGS. 7I-7L), Donor 2 (FIGS. 7E-7H), and Donor 3 (FIGS. 7M-7P).

### 6.4.2 Target 2

The ability of heterodimeric proteins specific for Target 2 to elicit IL-2 secretion by SEA-stimulated PBMCs was investigated.

Experiments were performed as described in Example 6.4.1 above utilizing BA115, BA116, BA117, BA118, and Reference Homodimeric Protein 1. A homodimeric isotype protein, which comprises S239D/A330L/I332E mutations (“Fc enhanced”), was used as an isotype control.

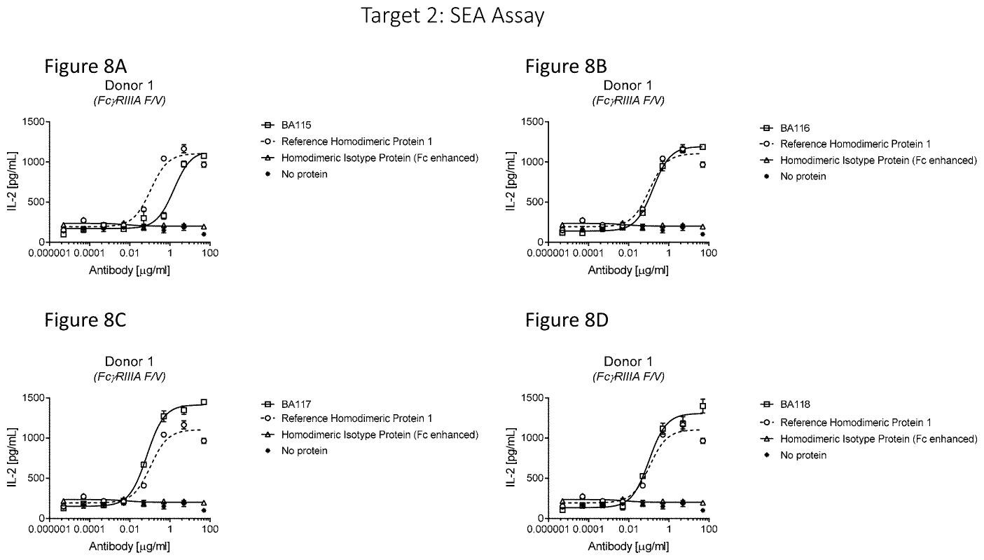

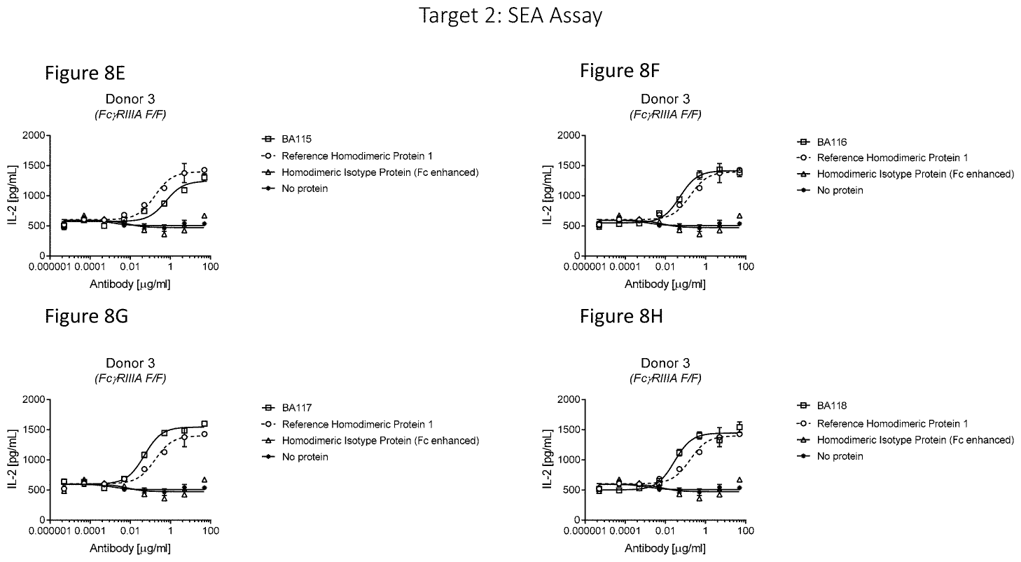

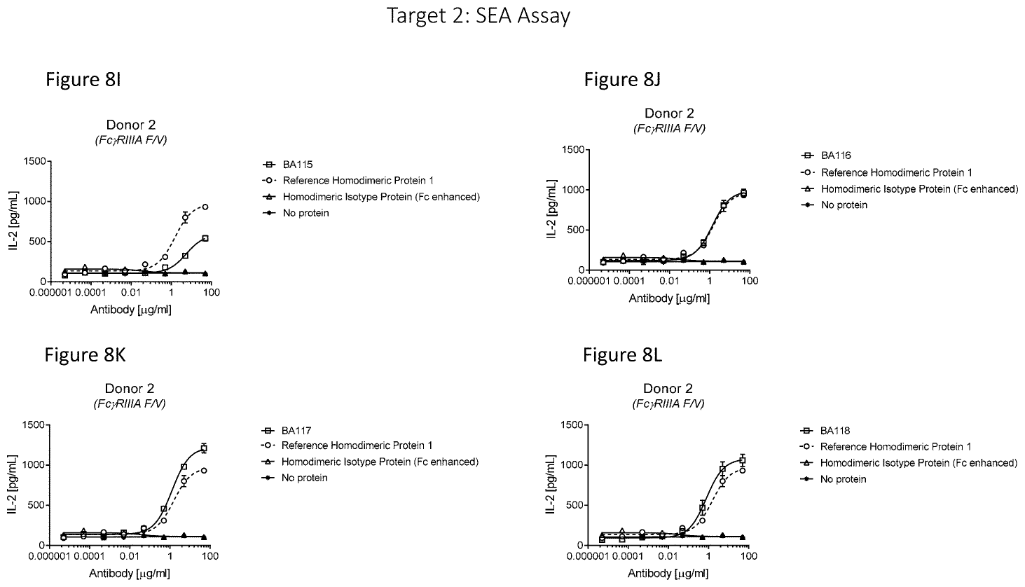

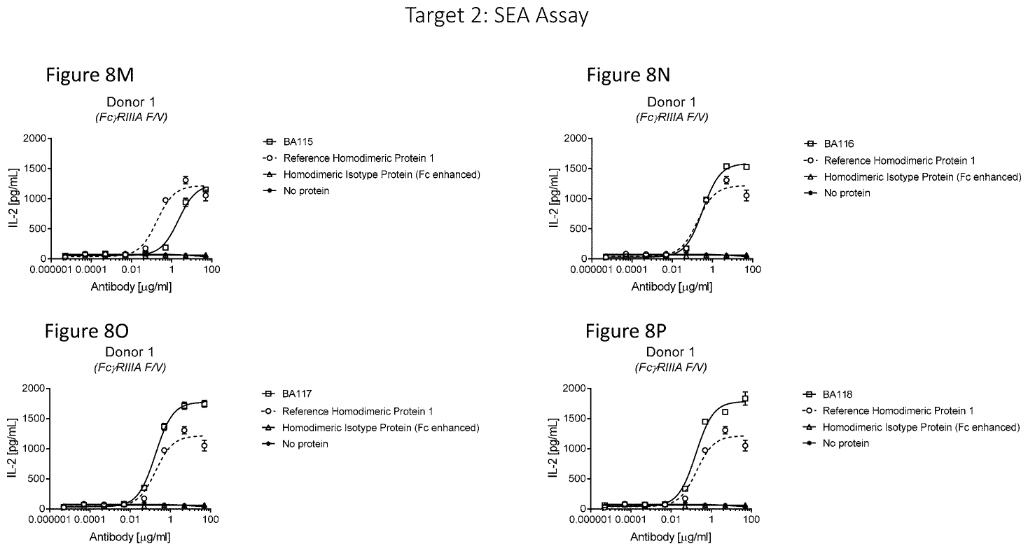

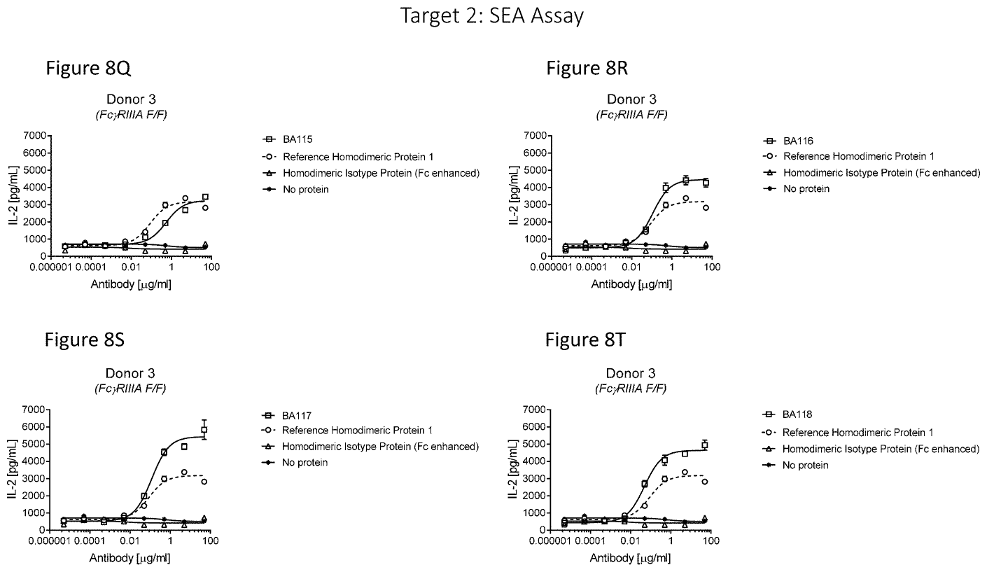

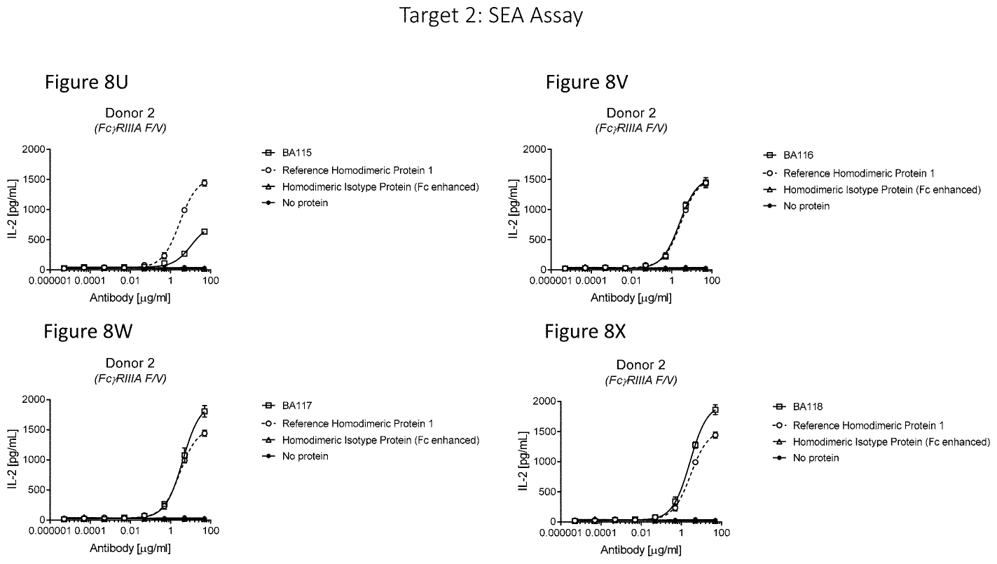

As shown in FIGS. 8A-8X, BA117 potently enhanced IL-2 secretion in a dose-dependent manner and demonstrated superior functional activity compared BA116 and BA115 and Reference Homodimeric Protein 1, also specific for Target 2. BA115 demonstrated significantly reduced potency compared to BA117, BA118 or BA116. BA115 and BA118 variant showed comparable functional activity at enhancing IL-2 secretion from SEA-stimulated PBMC donors.

### 6.5 Example 5:Fc Binding

### 6.5.1 FcγRIIIA (CD16) Binding

### 6.5.1.1 Target 1

The ability of BA111, BA112, BA113, and BA114 to bind FcγRIIIA was assessed using a cell binding assay.

Briefly, CHO cells expressing hFcγRIIIA (V/V) or hFcγRIIIA (F/F) were thawed from frozen and resuspended in 5ml FACS buffer (DPBS, BSA 0.5%, Azide 0.05%) at a density of 4×10⁶ cells/ml. The cells were dispensed in a 96-well U-bottom tissue culture plate at a density of 2×10⁵ cells/well and were then incubated for 45 minutes at 4° C. with a series dilution of BA111, BA112, BA113, and BA114 at concentrations from 60 μg/mL to 7 ng/mL diluted in FACS buffer. For antibody staining, the cells were washed twice with cold FACS buffer and re-suspended in FACS buffer containing goat anti-human IgG-PE (F(ab)2 anti-Fc) (JIR, Cat #109-116-098) at 1:800 dilution. After a 45-minute incubation at 4° C., the cells were washed twice with cold FACS buffer, and the cells were analyzed by flow cytometry (BD LSR Fortessa Flow Cytometer). The data were analyzed by the FlowJo software by sequentially gating on the FSC-A vs. SSC-A, SSC-H vs SSC-A, and Count vs PE. Geometric Mean fluorescence intensity (gMFI) values were calculated, and the data were plotted by GraphPad Prism software. Data is from three separate experiments; error bar represent standard error of the mean.

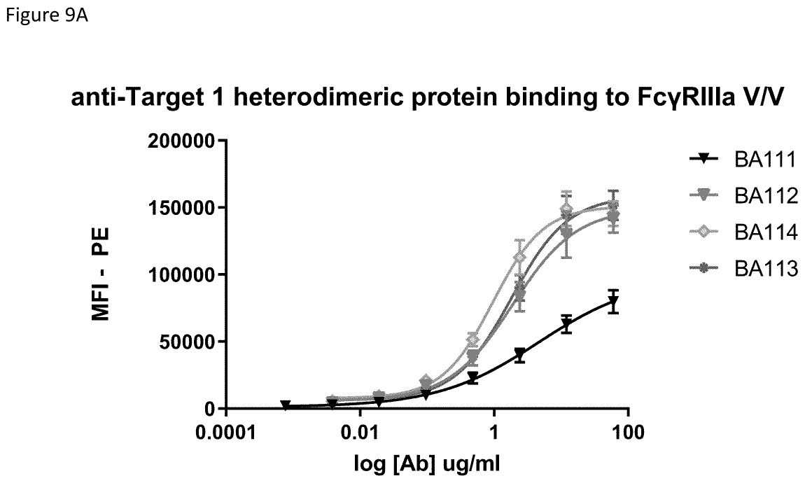

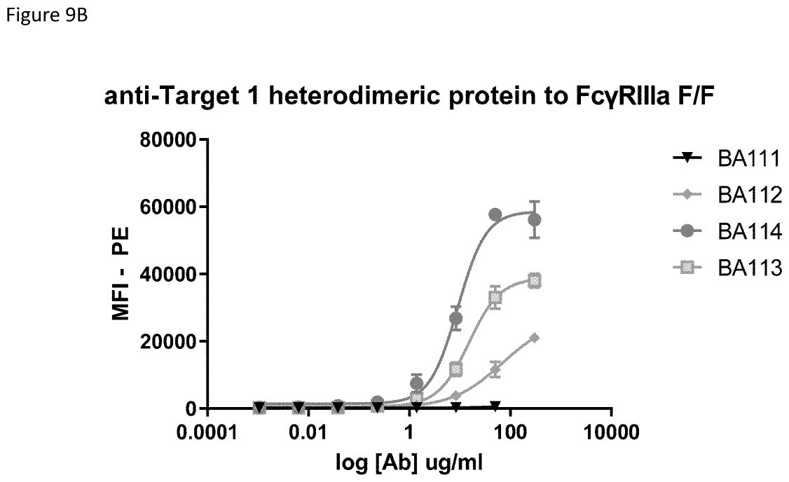

As shown in FIGS. 9A and 9B, BA112, BA113, and BA114 bind to the FcγRIIIA V/V (FIG. 9A) and F/F (FIG. 9B) expressed on CHO cells. BA113 demonstrates enhanced binding FcγRIIIA V/V (FIG. 9A) and F/F (FIG. 9B) as compared to BA111.

### 6.5.1.2 Target 2

The ability of BA115, BA116, BA117, and BA118 to bind FcγRIIIA was assessed using a cell binding assay.

The experiment was carried out as described in section 6.5.1.1 except that heterodimeric proteins BA115, BA116, BA117, and BA118 were used at concentrations from 60 μg/mL to 7 ng/mL. Data is from three experiments, error bar represent standard error of the mean.

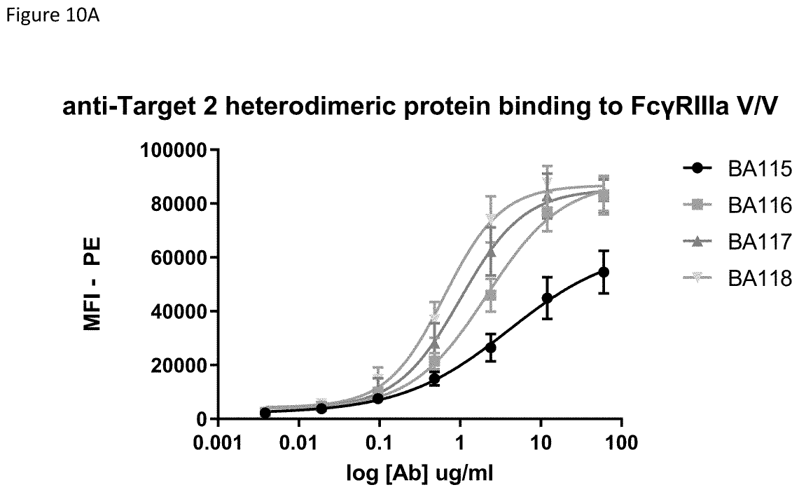

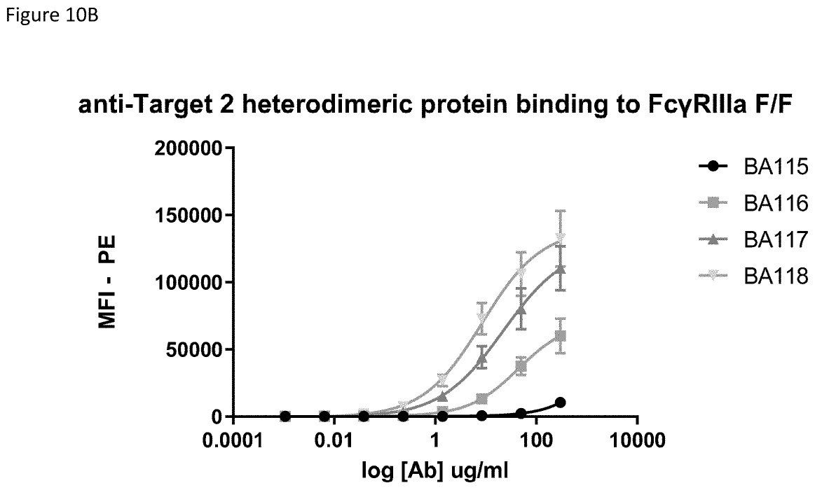

As shown in FIGS. 10A and 10B, BA116, BA117, and BA118 bind to the FcγRIIIA V/V (FIG. 10A) and F/F (FIG. 10B) expressed on CHO cells. BA117 demonstrates enhanced binding to FcγRIIIA V/V (FIG. 10A) and F/F (FIG. 10B) as compared to BA115.

### 6.5.2 Other Fc Receptors

The ability of BA112, BA113, BA114, BA115, BA116, BA117, and BA118 to bind to FcγRI, FcγRIIa H/H, FcγRIIa R/R, and FcγRIIb was also assessed using cell binding assays and no meaningful differences were observed.

### 6.5.3 FcγRIIIA (CD16) Binding for Additional Heterodimeric Proteins

This example describes the synthesis, heterodimerization, and Fc receptor binding of additional Fc polypeptides specific for Target 1 or Target 2 (two different antigens present on the surface of human T cells).

Additional Fc polypeptides (each comprising a half-antibody specific for either Target 1 or Target 2) were expressed in CHO cells and purified using Protein A affinity chromatography (GE Healthcare). The binding specificity of the antigen binding portion, and the amino acid sequence of the heavy chain constant region, for each of the twelve Fc polypeptides is described in Table 5.

TABLE 5
Fc polypeptides

| | | SEQ ID NO of |
|---|---|---|
| Fc | | heavy chain |
| polypeptide | | constant region |
| # | Antigen specificity and Fc mutation(s) | sequence* |
| 13 | Target 1 T366W/S239D/A330L | 68 |
| 14 | Target 1 T366W/S239D/I332E | 26 |
| 15 | Target 1 T366W/A330L/I332E | 75 |
| 16 | Target 1 T366W/S239D | 28 |
| 17 | Target 1 T366W/A330L | 69 |
| 18 | Target 1 T366W/I332E | 70 |
| 19 | Target 1 | 71 |
| | T366S/L368A/Y407V/S239D/A330L | |
| 20 | Target 1 | 72 |
| | T366S/L368A/Y407V/A330L/I332E | |
| 21 | Target 1 | 34 |
| | T366S/L368A/Y407V/S239D/I332E | |
| 22 | Target 1 T366S/L368A/Y407V/A330L | 73 |
| 23 | Target 1 T366S/L368A/Y407V/I332E | 74 |
| 24 | Target 2 T366W/S239D/A330L | 68 |
| 25 | Target 2 T366W/S239D/I332E | 26 |
| 26 | Target 2 T366W/S239D | 28 |
| 27 | Target 2 T366W/A330L | 69 |
| 28 | Target 2 T366W/I332E | 70 |
| 29 | Target 2 | 71 |
| | T366S/L368A/Y407V/S239D/A330L | |
| 30 | Target 2 | 72 |
| | T366S/L368A/Y407V/A330L/I332E | |
| 31 | Target 2 | 34 |
| | T366S/L368A/Y407V/S239D/I332E | |
| 32 | Target 2 T366S/L368A/Y407V/A330L | 73 |
| 33 | Target 2 T366S/L368A/Y407V/I332E | 74 |

The Fc polypeptides set forth in Tables 3 and/or 5 were heterodimerized to form the monospecific heterodimeric proteins described in Table 6. Heterodimerization was performed as described in Example 6.1.2.

TABLE 6
Heterodimeric proteins

| | First | Second |
|---|---|---|
| Name | Fc polypeptide | Fc polypeptide |
| BA115 | Fc polypeptide #7 | Fc polypeptide #8 |
| BA116 | Fc polypeptide #9 | Fc Polypeptide #8 |
| BA161 | Fc polypeptide #24 | Fc polypeptide #8 |
| BA162 | Fc polypeptide #25 | Fc Polypeptide #8 |
| BA163 | Fc polypeptide #26 | Fc polypeptide #8 |
| BA164 | Fc polypeptide #27 | Fc polypeptide #8 |
| BA165 | Fc polypeptide #28 | Fc polypeptide #8 |
| BA166 | Fc polypeptide #7 | Fc polypeptide #29 |
| BA167 | Fc polypeptide #9 | Fc polypeptide #29 |
| BA168 | Fc polypeptide #24 | Fc polypeptide #29 |
| BA169 | Fc polypeptide #25 | Fc polypeptide #29 |
| BA170 | Fc polypeptide #26 | Fc polypeptide #29 |
| BA171 | Fc polypeptide #27 | Fc polypeptide #29 |
| BA172 | Fc polypeptide #28 | Fc polypeptide #29 |
| BA173 | Fc polypeptide #7 | Fc polypeptide #30 |
| BA174 | Fc polypeptide #9 | Fc polypeptide #30 |
| BA175 | Fc polypeptide #24 | Fc polypeptide #30 |
| BA176 | Fc polypeptide #25 | Fc polypeptide #30 |
| BA177 | Fc polypeptide #26 | Fc polypeptide #30 |
| BA178 | Fc polypeptide #27 | Fc polypeptide #30 |
| BA179 | Fc polypeptide #28 | Fc polypeptide #30 |
| BA180 | Fc polypeptide #7 | Fc polypeptide #31 |
| BA181 | Fc polypeptide #9 | Fc polypeptide #31 |
| BA182 | Fc polypeptide #24 | Fc polypeptide #31 |
| BA183 | Fc polypeptide #25 | Fc polypeptide #31 |
| BA184 | Fc polypeptide #26 | Fc polypeptide #31 |
| BA185 | Fc polypeptide #27 | Fc polypeptide #31 |
| BA186 | Fc polypeptide #28 | Fc polypeptide #31 |
| BA187 | Fc polypeptide #7 | Fc polypeptide #11 |
| BA117 | Fc polypeptide #9 | Fc polypeptide #11 |
| BA188 | Fc polypeptide #24 | Fc polypeptide #11 |
| BA189 | Fc polypeptide #25 | Fc polypeptide #11 |
| BA190 | Fc polypeptide #26 | Fc polypeptide #11 |
| BA191 | Fc polypeptide #27 | Fc polypeptide #11 |
| BA192 | Fc polypeptide #28 | Fc polypeptide #11 |
| BA193 | Fc polypeptide #7 | Fc polypeptide #32 |
| BA194 | Fc polypeptide #9 | Fc polypeptide #32 |
| BA195 | Fc polypeptide #24 | Fc polypeptide #32 |
| BA196 | Fc polypeptide #25 | Fc polypeptide #32 |
| BA197 | Fc polypeptide #26 | Fc polypeptide #32 |
| BA198 | Fc polypeptide #27 | Fc polypeptide #32 |
| BA199 | Fc polypeptide #28 | Fc polypeptide #32 |
| BA200 | Fc polypeptide #7 | Fc polypeptide #33 |
| BA201 | Fc polypeptide #9 | Fc polypeptide #33 |
| BA202 | Fc polypeptide #24 | Fc polypeptide #33 |
| BA203 | Fc polypeptide #25 | Fc polypeptide #33 |
| BA204 | Fc polypeptide #26 | Fc polypeptide #33 |
| BA205 | Fc polypeptide #27 | Fc polypeptide #33 |
| BA206 | Fc polypeptide #28 | Fc polypeptide #33 |
| BA111 | Fc polypeptide #1 | Fc polypeptide #2 |
| BA112 | Fc polypeptide #3 | Fc polypeptide #2 |
| BA207 | Fc polypeptide #13 | Fc polypeptide #2 |
| BA208 | Fc polypeptide #14 | Fc polypeptide #2 |
| BA209 | Fc polypeptide #15 | Fc polypeptide #2 |
| BA210 | Fc polypeptide #16 | Fc polypeptide #2 |
| BA211 | Fc polypeptide #17 | Fc polypeptide #2 |
| BA212 | Fc polypeptide #18 | Fc polypeptide #2 |
| BA213 | Fc polypeptide #1 | Fc polypeptide #19 |
| BA214 | Fc polypeptide #3 | Fc polypeptide #19 |
| BA215 | Fc polypeptide #13 | Fc polypeptide #19 |
| BA216 | Fc polypeptide #14 | Fc polypeptide #19 |
| BA217 | Fc polypeptide #15 | Fc polypeptide #19 |
| BA218 | Fc polypeptide #16 | Fc polypeptide #19 |
| BA219 | Fc polypeptide #17 | Fc polypeptide #19 |
| BA220 | Fc polypeptide #18 | Fc polypeptide #19 |
| BA221 | Fc polypeptide #1 | Fc polypeptide #20 |
| BA222 | Fc polypeptide #3 | Fc polypeptide #20 |
| BA223 | Fc polypeptide #13 | Fc polypeptide #20 |
| BA224 | Fc polypeptide #14 | Fc polypeptide #20 |
| BA225 | Fc polypeptide #15 | Fc polypeptide #20 |
| BA226 | Fc polypeptide #16 | Fc polypeptide #20 |
| BA227 | Fc polypeptide #17 | Fc polypeptide #20 |
| BA228 | Fc polypeptide #18 | Fc polypeptide #20 |
| BA229 | Fc polypeptide #1 | Fc Polypeptide #21 |
| BA230 | Fc polypeptide #3 | Fc Polypeptide #21 |
| BA231 | Fc polypeptide #13 | Fc Polypeptide #21 |
| BA232 | Fc polypeptide #14 | Fc Polypeptide #21 |
| BA233 | Fc polypeptide #15 | Fc Polypeptide #21 |
| BA234 | Fc polypeptide #16 | Fc Polypeptide #21 |
| BA235 | Fc polypeptide #17 | Fc Polypeptide #21 |
| BA236 | Fc polypeptide #18 | Fc Polypeptide #21 |
| BA237 | Fc polypeptide #1 | Fc polypeptide #5 |
| BA113 | Fc polypeptide #3 | Fc polypeptide #5 |
| BA238 | Fc polypeptide #13 | Fc polypeptide #5 |
| BA239 | Fc polypeptide #14 | Fc polypeptide #5 |
| BA240 | Fc polypeptide #15 | Fc polypeptide #5 |
| BA241 | Fc polypeptide #16 | Fc polypeptide #5 |
| BA242 | Fc polypeptide #17 | Fc polypeptide #5 |
| BA243 | Fc polypeptide #18 | Fc polypeptide #5 |
| BA244 | Fc polypeptide #1 | Fc polypeptide #22 |
| BA245 | Fc polypeptide #3 | Fc polypeptide #22 |
| BA246 | Fc polypeptide #13 | Fc polypeptide #22 |
| BA247 | Fc polypeptide #14 | Fc polypeptide #22 |
| BA248 | Fc polypeptide #15 | Fc polypeptide #22 |
| BA249 | Fc polypeptide #16 | Fc polypeptide #22 |
| BA250 | Fc polypeptide #17 | Fc polypeptide #22 |
| BA251 | Fc polypeptide #18 | Fc polypeptide #22 |
| BA252 | Fc polypeptide #1 | Fc polypeptide #23 |
| BA253 | Fc polypeptide #3 | Fc polypeptide #23 |
| BA254 | Fc polypeptide #13 | Fc polypeptide #23 |
| BA255 | Fc polypeptide #14 | Fc polypeptide #23 |
| BA256 | Fc polypeptide #15 | Fc polypeptide #23 |
| BA257 | Fc polypeptide #16 | Fc polypeptide #23 |
| BA258 | Fc polypeptide #17 | Fc polypeptide #23 |
| BA259 | Fc polypeptide #18 | Fc polypeptide #23 |

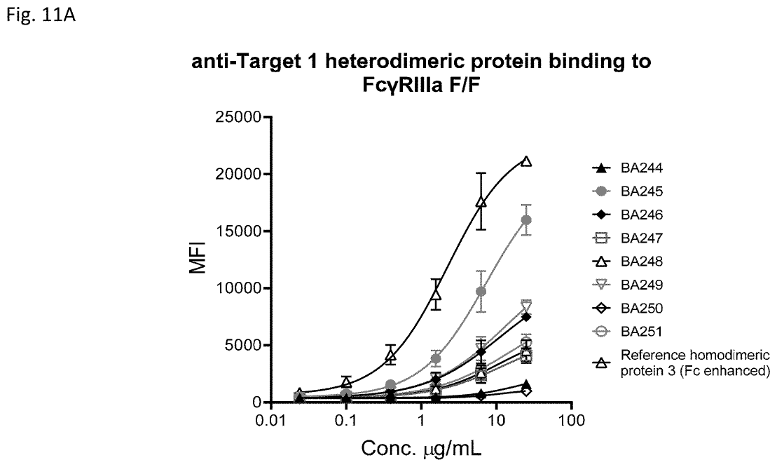

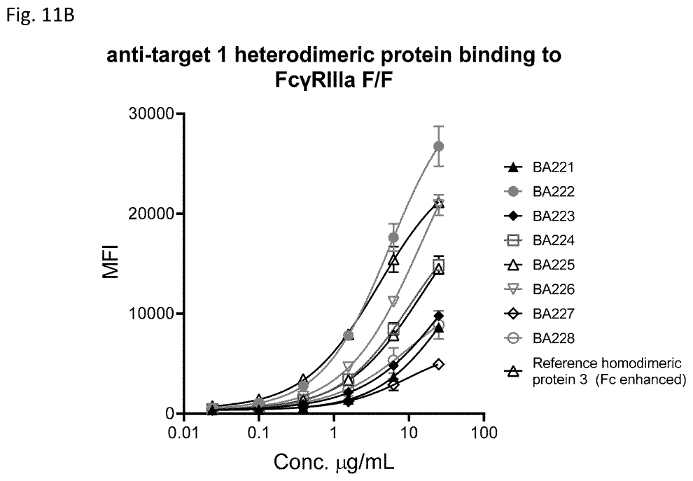

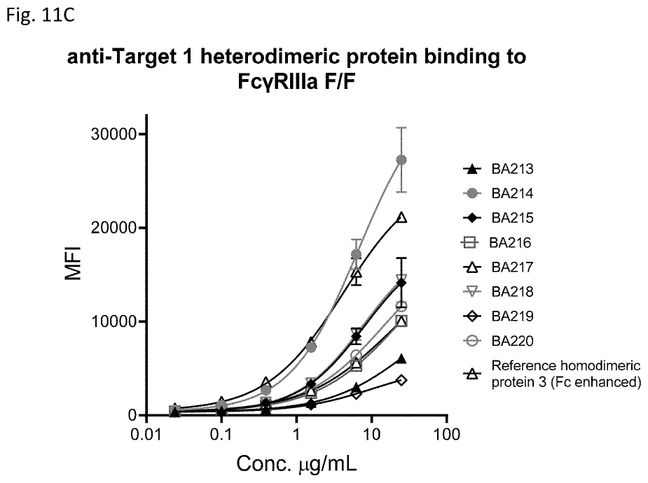

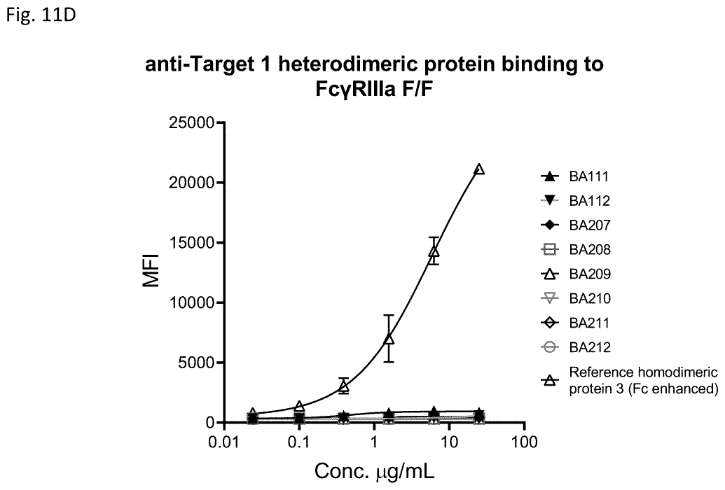

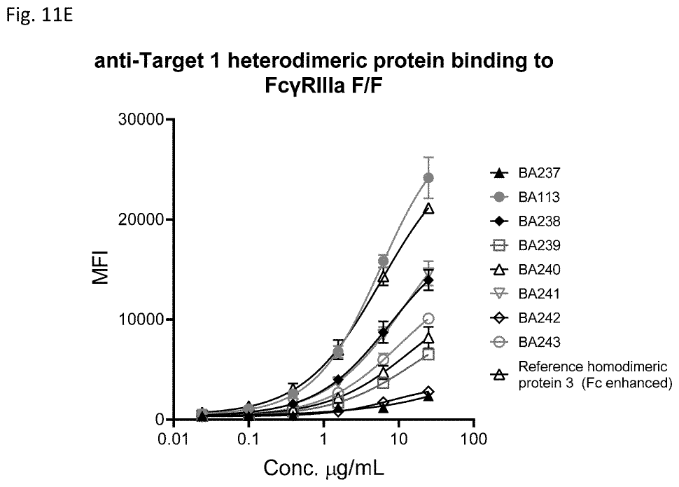

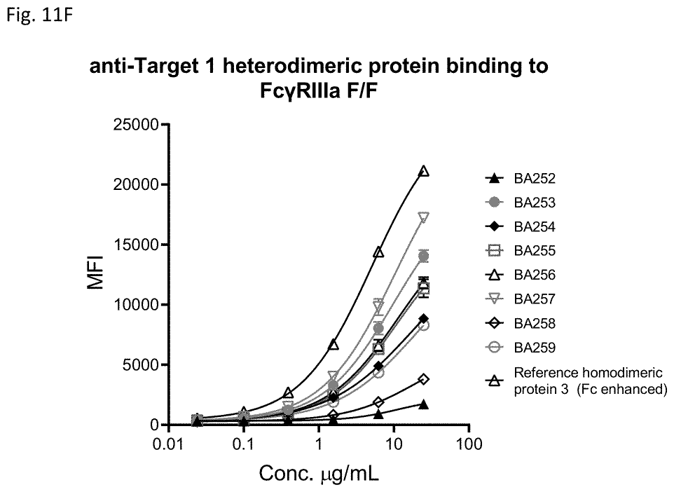

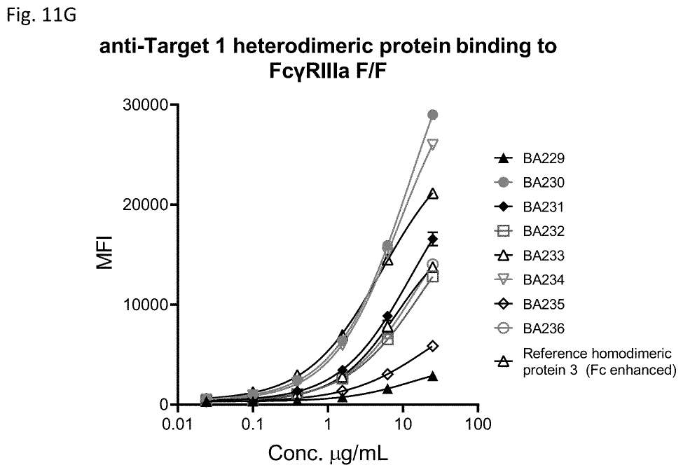

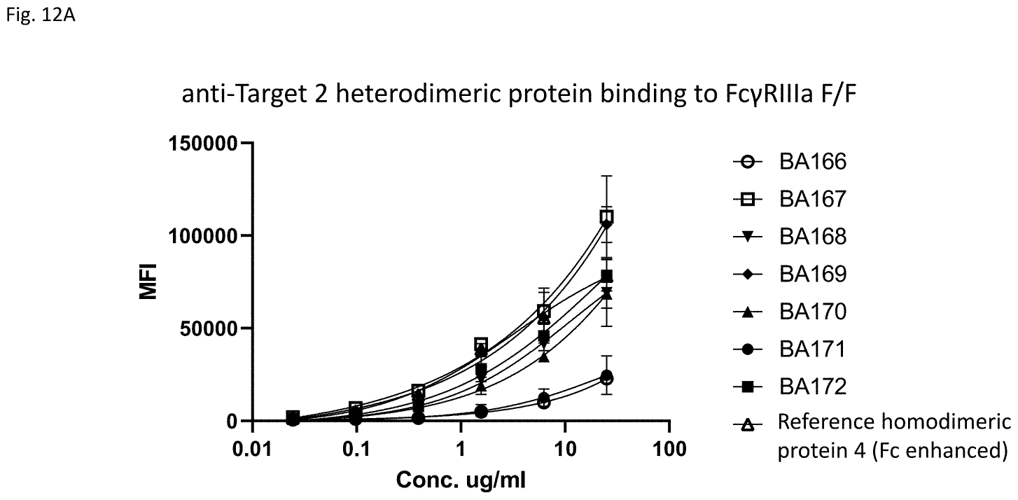

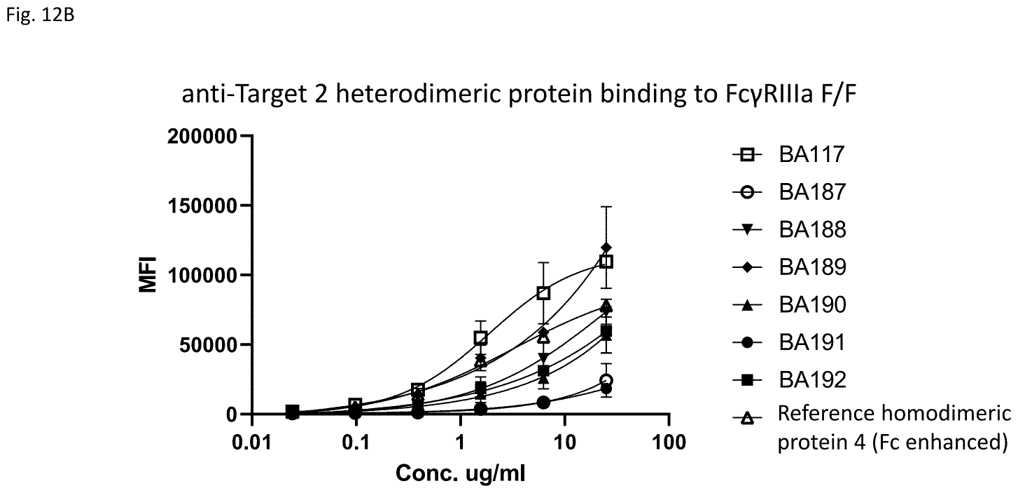

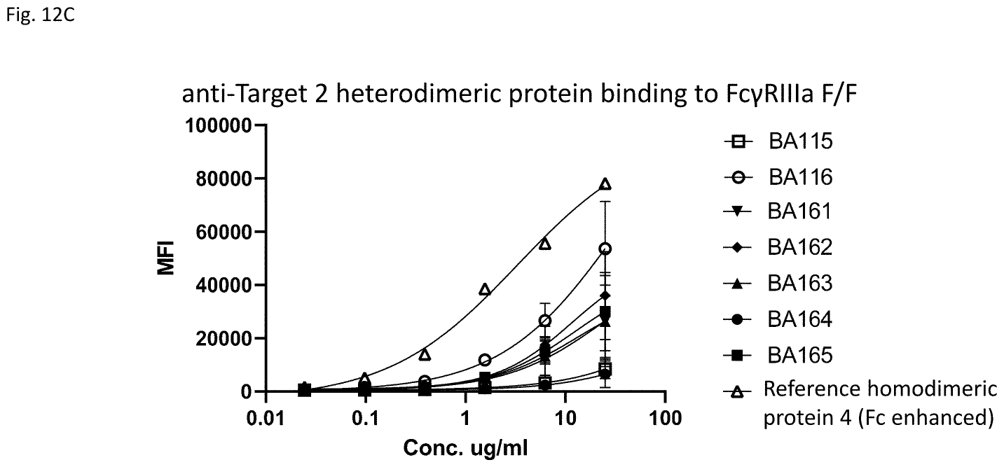

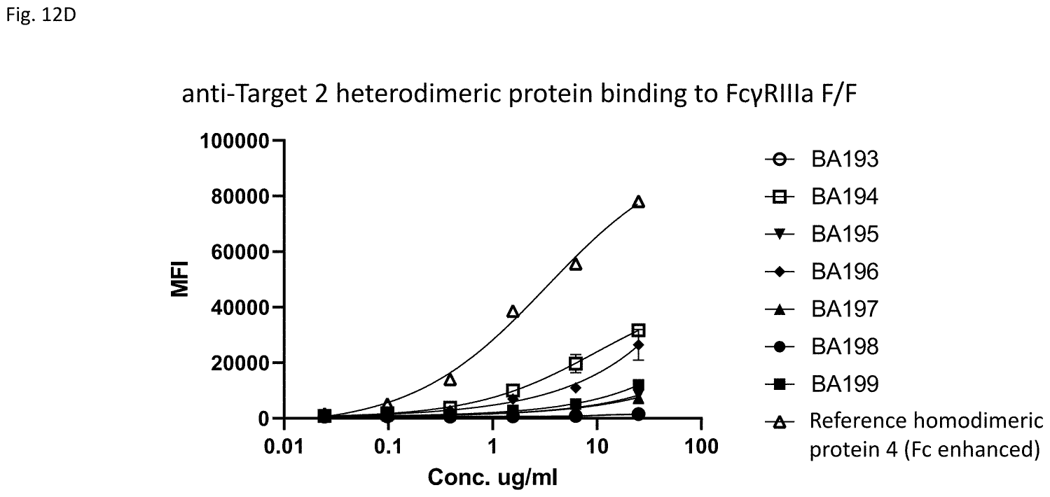

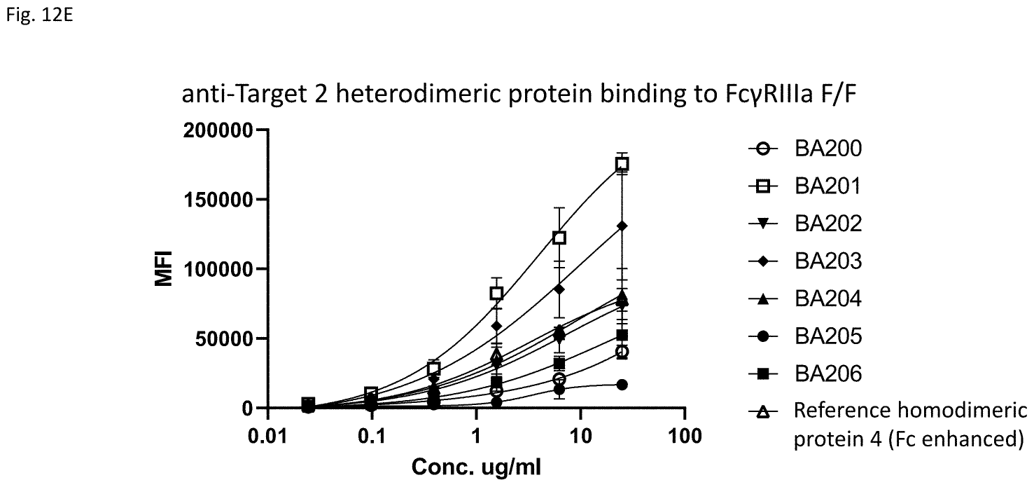

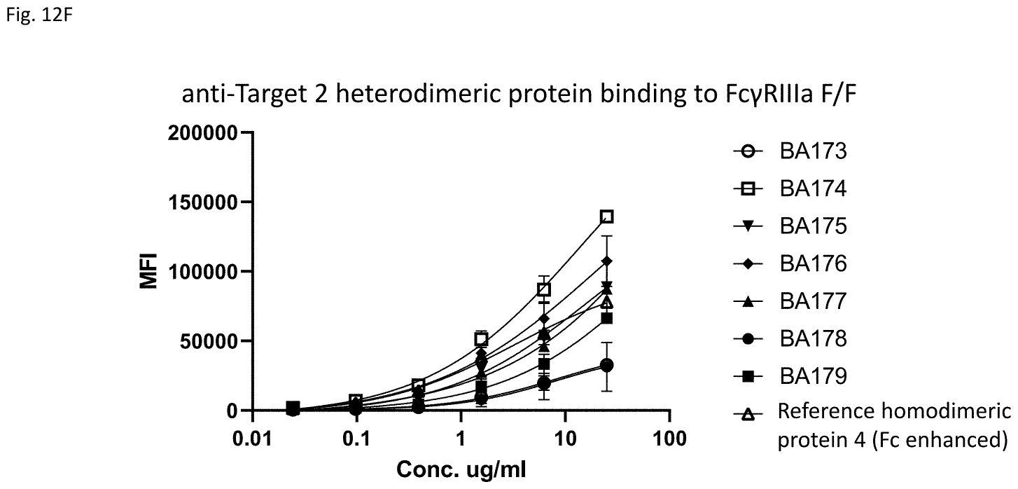

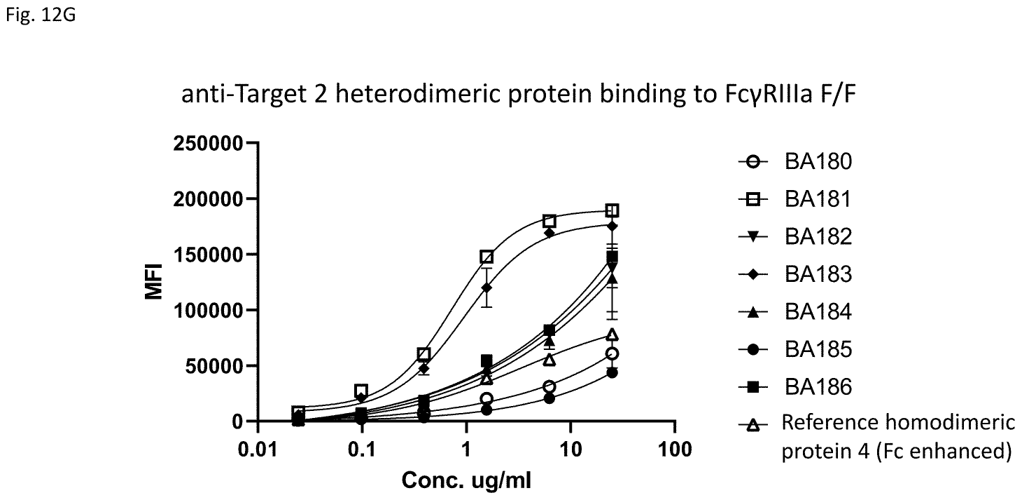

The ability of the heterodimeric proteins in Table 6 to bind FcγRIIIA F/F was assessed using a cell binding assay, as described in Example 6.5.1 with the exception that the detection antibody used for assessing the anti-target 1 heterodimeric proteins shown in FIG. 11 and Table 7 was goat anti-human IgG-Alexa Fluor 488, Fcγ Fragment Specific (JIR, Cat# 109-546-098). The dilution range used in both FIG. 11 and FIG. 12 was 100m/ml-0 μg/ml with a 1:4 stepwise dilution. In each case, a reference homodimeric protein containing S239D/A330L/I332E mutations (“Fc enhanced”) was used for comparison.

The ability of the anti-target 1 heterodimeric proteins with various Fc polypeptides to bind to CHO cells expressing cell surface human FcγRIIIA F/F is shown in FIGS. 11A-11G. The level of heterodimeric protein binding to the cells, as assessed by the geometric mean fluorescence intensity (MFI), was plotted against the concentrations of heterodimeric protein incubated with the cells. The area under the curve for each heterodimeric protein is presented in Table 7.

The ability of the anti-target 2 heterodimeric proteins with various Fc polypeptides to bind to CHO cells expressing cell surface human FcγRIIIA F/F is shown in FIGS. 12A-12G. The level of heterodimeric protein binding to the cells, as assessed by the geometric mean fluorescence intensity (MFI), was plotted against the concentrations of heterodimeric protein incubated with the cells. The area under the curve for each heterodimeric protein is presented in Table 8.

TABLE 7
Area under the curve for heterodimeric
proteins presented in FIGS. 11A-11G

| Name | AUC |
|---|---|
| Reference homodimeric | 400560 |
| protein 3 | |
| BA222 | 482384 |
| BA214 | 480264 |
| BA230 | 479341 |
| BA232 | 440787 |
| BA113 | 434339 |
| BA224 | 341586 |
| BA255 | 289244 |
| BA245 | 276263 |
| BA233 | 270566 |
| BA226 | 249901 |
| BA239 | 248715 |
| BA216 | 248277 |
| BA240 | 246156 |
| BA217 | 242282 |
| BA223 | 238873 |
| BA253 | 236702 |
| BA231 | 229864 |
| BA236 | 224726 |
| BA234 | 205394 |
| BA254 | 196874 |
| BA220 | 193500 |
| BA257 | 188615 |
| BA243 | 173798 |
| BA215 | 169206 |
| BA218 | 164197 |
| BA225 | 155132 |
| BA228 | 153773 |
| BA256 | 147779 |
| BA247 | 140591 |
| BA238 | 139565 |
| BA259 | 135178 |
| BA221 | 129190 |
| BA248 | 128792 |
| BA241 | 109967 |
| BA213 | 97090 |
| BA235 | 95446 |
| BA251 | 88508 |
| BA227 | 84590 |
| BA246 | 76963 |
| BA249 | 69589 |
| BA219 | 66111 |
| BA258 | 60553 |
| BA242 | 49501 |
| BA229 | 48590 |
| BA237 | 40479 |
| BA252 | 28796 |
| BA244 | 26352 |
| BA111 | 22945 |
| BA250 | 17687 |
| BA209 | 12160 |
| BA112 | 11542 |
| BA208 | 8967 |
| BA211 | 8941 |
| BA207 | 8588 |
| BA212 | 8440 |
| BA210 | 8416 |

TABLE 8
Area under the curve for heterodimeric
proteins presented in FIGS. 12A-12G

| Name | AUC |
|---|---|
| Reference homodimeric | 92261 |
| protein 4 | |
| BA181 | 409269 |
| BA183 | 363045 |
| BA201 | 298944 |
| BA174 | 229726 |
| BA186 | 226029 |
| BA203 | 221615 |
| BA182 | 213536 |
| BA184 | 201835 |
| BA117 | 201821 |
| BA176 | 175408 |
| BA167 | 175293 |
| BA189 | 173119 |
| BA169 | 163892 |
| BA175 | 138038 |
| BA204 | 136837 |
| BA177 | 131142 |
| BA172 | 128775 |
| BA202 | 123520 |
| BA168 | 110262 |
| BA188 | 109496 |
| BA170 | 100200 |
| BA179 | 98624 |
| BA180 | 90331 |
| BA192 | 89857 |
| BA206 | 83706 |
| BA116 | 79878 |
| BA190 | 78765 |
| BA200 | 63320 |
| BA185 | 63295 |
| BA173 | 55475 |
| BA162 | 50986 |
| BA194 | 49928 |
| BA165 | 46970 |
| BA178 | 46437 |
| BA163 | 41201 |
| BA161 | 38733 |
| BA196 | 37758 |
| BA171 | 37697 |
| BA187 | 31952 |
| BA166 | 31226 |
| BA205 | 30148 |
| BA191 | 27189 |
| BA199 | 18921 |
| BA115 | 14042 |
| BA195 | 12935 |
| BA197 | 12766 |
| BA164 | 11045 |
| BA193 | 3647 |
| BA198 | 3474 |

The invention is not to be limited in scope by the specific embodiments described herein. Indeed, various modifications disclosed herein in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A heterodimeric protein comprising a first Fc polypeptide and a second Fc polypeptide, wherein:
a) the first Fc polypeptide comprises aspartate, glutamate, and tryptophan at amino acid positions 239, 332, and 366, respectively; and
b) the second Fc polypeptide comprises aspartate, serine, alanine, and valine at amino acid positions 239, 366, 368, and 407, respectively, but does not comprise glutamate at amino acid position 332,
wherein the amino acid positions are numbered according to the EU index.

2. The heterodimeric protein of claim 1, wherein:
a) the first Fc polypeptide further comprises leucine at amino acid position 330; and/or
b) the second Fc polypeptide does not comprise leucine at amino acid position 330,
wherein the amino acid positions are numbered according to the EU index.

3. (canceled)

4. The heterodimeric protein of claim 1, wherein the first Fc polypeptide comprises a first antigen-binding moiety and/or the second Fc polypeptide comprises a second antigen-binding moiety.

5. The heterodimeric protein of claim 4, wherein the first and/or second antigen-binding moiety comprises an antibody variable domain, an extracellular domain of a cell surface receptor, a soluble T cell receptor, or a ligand, optionally wherein:
a) the first and/or second antigen-binding moiety comprises a VH, a VL, a VHH, a VH/VL pair, an scFv, a diabody, and/or a Fab;
b) the cell surface receptor is a tumor necrosis factor superfamily receptor, a vascular endothelial growth factor receptor, or a transforming growth factor receptor; or
c) the ligand is a hormone or growth factor,
wherein the first and second antigen-binding moieties specifically bind to the same or different target molecules.

6-9. (canceled)

10. The heterodimeric protein of claim 1, wherein the first and/or second Fc polypeptide comprises a CH1 domain, hinge region, CH2 domain and/or CH3 domain of human IgG₁, IgG₂, IgG₃, IgG₄, IgA₁, or IgA₂.

11. The heterodimeric protein of claim 10, wherein the first and/or second Fc polypeptide comprises an antibody heavy chain, optionally wherein:
a) the antibody heavy chain lacks a CH1 domain and/or a portion of a hinge region; or
b) the antibody heavy chain is a full-length antibody heavy chain,
optionally wherein the antibody heavy chain is a human IgG₁, IgG₂, IgG₃, IgG₄, IgA₁, or IgA₂ heavy chain,
optionally wherein the first and/or second Fc polypeptide further comprises an antibody light chain, optionally wherein the antibody light chain is a human kappa or lambda light chain.

12-16. (canceled)

17. The heterodimeric protein of claim 1, wherein:
a) the first Fc polypeptide comprises a first half-antibody comprising a first antibody heavy chain and a first antibody light chain;
b) the second Fc polypeptide comprises a second half-antibody comprising a second antibody heavy chain and a second antibody light chain; or
c) the first Fc polypeptide comprises a first half-antibody comprising a first antibody heavy chain and a first antibody light chain and the second Fc polypeptide comprises a second half-antibody comprising a second antibody heavy chain and a second antibody light chain,
optionally wherein the first and second half-antibodies bind to different target molecules or to different regions of the same target molecule.

18-19. (canceled)

20. The heterodimeric protein of claim 1, wherein:
a) the first Fc polypeptide comprises an amino acid sequence that is at least 75% identical (optionally at least 76, 77, 78, 79, 80, 81, 82, 83, 84, 85, 86, 87, 88, 89, 90, 91, 92, 93, 94, 95, 96, 97, 98, 99, or 100% identical) to an amino acid sequence selected from the group consisting of SEQ ID NO: 1-3, 6-7, 10-11, 24-27, 48-51, and 62-65; and/or
b) the second Fc polypeptide comprises an amino acid sequence that is at least 75% identical (optionally at least 75, 76, 77, 78, 79, 80, 81, 82, 83, 84, 85, 86, 87, 88, 89, 90, 91, 92, 93, 94, 95, 96, 97, 98, 99, or 100% identical) to an amino acid sequence selected from the group consisting of SEQ ID NO: 1-3, 5, 12-13, 36-37, 60-61, and 66-67, or
c) the first Fc polypeptide comprises an amino acid sequence that is at least 75% identical (optionally at least 76, 77, 78, 79, 80, 81, 82, 83, 84, 85, 86, 87, 88, 89, 90, 91, 92, 93, 94, 95, 96, 97, 98, 99, or 100% identical) to an amino acid sequence selected from the group consisting of SEQ ID NO: 1-3, 6-7, 10-11, 24-27, 48-51, and 62-65; and the second Fc polypeptide comprises an amino acid sequence that is at least 75% identical (optionally at least 75, 76, 77, 78, 79, 80, 81, 82, 83, 84, 85, 86, 87, 88, 89, 90, 91, 92, 93, 94, 95, 96, 97, 98, 99, or 100% identical) to an amino acid sequence selected from the group consisting of SEQ ID NO: 1-3, 5, 12-13, 36-37, 60-61, and 66-67.

21. (canceled)

22. The heterodimeric protein of claim 1, wherein the first Fc polypeptide and second Fc polypeptide comprise amino acid sequences that are at least 75% identical (optionally at least 75, 76, 77, 78, 79, 80, 81, 82, 83, 84, 85, 86, 87, 88, 89, 90, 91, 92, 93, 94, 95, 96, 97, 98, 99, or 100% identical) to the amino acid sequences of SEQ ID NOs: 24 and 36; 24 and 37; 25 and 36; 25 and 37; 26 and 36; 26 and 37; 27 and 36; 27 and 37; 48 and 60; 48 and 61; 49 and 60; 49 and 61; 50 and 60; 50 and 61; 51 and 60; 51 and 61; 62 and 66; 62 and 67; 63 and 66; 63 and 67; 64 and 66; 64 and 67; 65 and 66; or 65 and 67, respectively.

23. The heterodimeric protein of claim 1, wherein the first Fc polypeptide and the second Fc polypeptide comprises the amino acid sequences set forth in SEQ ID NOs: 24 and 36; 24 and 37; 25 and 36; 25 and 37; 26 and 36; 26 and 37; 27 and 36; 27 and 37; 48 and 60; 48 and 61; 49 and 60; 49 and 61; 50 and 60; 50 and 61; 51 and 60; 51 and 61; 62 and 66; 62 and 67; 63 and 66; 63 and 67; 64 and 66; 64 and 67; 65 and 66; or 65 and 67, respectively.

24-46. (canceled)

47. A pharmaceutical composition comprising the heterodimeric protein of claim 1 and a pharmaceutically acceptable carrier or excipient.

48. An isolated polynucleotide encoding the first and the second Fc polypeptides of the heterodimeric protein of any one of claim 1.

49. A vector comprising the polynucleotide of claim 48.

50. A recombinant host cell comprising:
a) a polynucleotide encoding the first and the second Fc polypeptides of the heterodimeric protein of any one of claim 1;
b) a vector comprising a polynucleotide encoding the first and the second Fc polypeptides of the heterodimeric protein of any one of claim 1;
c) a first polynucleotide encoding the first Fc polypeptide of the heterodimeric protein of claim 1, and a second polynucleotide encoding the second Fc polypeptide of the heterodimeric protein of claim 1; or
d) a first vector comprising a first polynucleotide encoding the first Fc polypeptide of the heterodimeric protein of claim 1, and a second vector comprising a second polynucleotide encoding the second Fc polypeptide of the heterodimeric protein of claim 1.

51. A method of producing a heterodimeric protein, the method comprising expressing in a cell:
a) a first polynucleotide encoding the first Fc polypeptide of the heterodimeric protein of claim 1; and
b) a second polynucleotide encoding the second Fc polypeptide of the heterodimeric protein of claim 1,
under conditions whereby the heterodimeric protein is produced.

52. A method of producing a heterodimeric protein, the method comprising expressing in a cell:
a) a first polynucleotide encoding the first antibody heavy chain of the heterodimeric protein of claim 17;
b) a second polynucleotide encoding the second antibody heavy chain of the heterodimeric protein of claim 17;
c) a third polynucleotide encoding the first antibody light chain of the heterodimeric protein of claim 17; and
d) a fourth polynucleotide encoding the second antibody light chain of the heterodimeric protein of claim 17,
under conditions whereby the heterodimeric protein is produced.

53. A method of producing a heterodimeric protein, the method comprising:
a) expressing in a first cell a first polynucleotide encoding the first Fc polypeptide of the heterodimeric protein of claim 1, under conditions whereby the first Fc polypeptide is produced;
b) expressing in a second cell a second polynucleotide encoding the second Fc polypeptide of the heterodimeric protein of claim 1, under conditions whereby the second Fc polypeptide is produced; and
c) contacting the first and the second Fc polypeptides produced in steps (a) and (b), under conditions whereby the first Fc polypeptide and the second Fc polypeptide heterodimerize to produce the heterodimeric protein.

54. A method of producing a heterodimeric, the method comprising:
a) expressing in a first cell a first polynucleotide encoding the first antibody heavy chain of the heterodimeric protein of claim 17 and second polynucleotide encoding the first antibody light chain of the heterodimeric protein of claim 17, under conditions whereby the first Fc polypeptide is produced;
b) expressing in a second cell a third polynucleotide encoding the second antibody heavy chain of the heterodimeric protein of claim 17, and fourth polynucleotide encoding the second antibody light chain of the heterodimeric protein of claim 17, under conditions whereby the second Fc polypeptide is produced; and
c) contacting the first and the second Fc polypeptides produced in steps (a) and (b), under conditions whereby the first Fc polypeptide and the second Fc polypeptide heterodimerize to produce the heterodimeric protein.

55. A method of producing a heterodimeric protein, the method comprising contacting the first Fc polypeptide and the second Fc polypeptide of the heterodimeric protein of claim 1 under conditions whereby the first Fc polypeptide and the second Fc polypeptide heterodimerize to produce the heterodimeric protein.

56. A recombinant host cell comprising:
a) a first polynucleotide encoding the first antibody heavy chain of the heterodimeric protein of claim 17, and a second polynucleotide encoding the second antibody heavy chain of the heterodimeric protein of claim 17, and optionally a third polynucleotide encoding the first antibody light chain of the heterodimeric protein of claim 17, and/or a fourth polynucleotide encoding the second antibody light chain of the heterodimeric protein of claim 17; or
b) a first vector comprising a first polynucleotide encoding the first antibody heavy chain of the heterodimeric protein of claim 17, and a second vector comprising a second polynucleotide encoding the second antibody heavy chain of the heterodimeric protein of claim 17, and optionally a third vector comprising a third polynucleotide encoding the first antibody light chain of the heterodimeric protein of claim 17, and/or a fourth vector comprising a fourth polynucleotide encoding the second antibody light chain of the heterodimeric protein of claim 17.
